(19) United States
(12) Patent Application Publication
(10) Pub. No.: US 2023/0416833 A1
(43) Pub. Date: Dec. 28, 2023

(54) SYSTEMS AND METHODS FOR MONITORING OF CANCER USING MINIMAL RESIDUAL DISEASE ANALYSIS

(71) Applicant: Predicine, Inc., Hayward, CA (US)

(72) Inventors: Shidong JIA, Palo Alto, CA (US); Pan DU, Dublin, CA (US)

(21) Appl. No.: 18/105,113

(22) Filed: Feb. 2, 2023

Related U.S. Application Data
(60) Provisional application No. 63/306,466, filed on Feb. 3, 2022.
(60) Provisional application No. 63/306,468, filed on Feb. 3, 2022.
(60) Provisional application No. 63/354,938, filed on Jun. 23, 2022.

(51) Int. Cl. C12Q 1/6886; C12Q 1/6869; G16B 30/00

(52) U.S. Cl. CPC C12Q 1/6886; C12Q 1/6869; G16B 30/00

(57) ABSTRACT

Provided herein are methods and systems monitoring of cancer using minimal residual disease analysis. The methods may comprise assaying multiple nucleic acids to detect a set of biomarkers from samples. The methods may comprise sequencing of nucleic acids. The method may comprise the generation of a probe panel. The methods may comprise processing the set of biomarkers to determine the presence of a cancer or cancer parameters. The processing may be performed by an algorithm.

### CROSS-REFERENCE

This application claims the benefit of U.S. Provisional Patent Application No. 63/306,466, filed Feb. 3, 2022, U.S. Provisional Patent Application No. 63/306,468, filed Feb. 3, 2022, and U.S. Provisional Patent Application No. 63/354,938, filed Jun. 23, 2022, all of which are incorporated by reference herein in its entirety.

### BACKGROUND

Cancer is a leading cause of deaths worldwide. Detection of cancer in individuals may be critical for providing treatment and improving patient outcomes. Cancer may be caused by genetic aberration which may lead to unregulated growth of calls. Detection of the genetic aberrations may be important for the detection of cancer. Sequencing of nucleic acids in a sample from a patient may be used to detect genetic aberrations.

### SUMMARY

Provided herein are systems and methods for detection of the presence or absence of cancer in a subject. The systems and methods provided herein comprises assaying polynucleotides to identify biomarkers of cancers in a subject. Detection of a type of cancer or the specific biomarkers for a given cancer may allow an effective treatment to be provided to an individual and may result in improved outcomes. For multiple types of cancer, the particular biomarkers that indicate a particular cancer type (or subtype) may be used to identify a prognosis for an individual suffering from the cancer. In order to provide accurate detection and prognosis for a cancer, multiple analytes may be examined. By analyzing an increased number of analytes (and sets of biomarkers from the analytes), the detection of a cancer (or cancer parameter) may be improved and may allow for the recommendation of an effective treatment, and may also allow for the prognosis to be more accurate.

In an aspect, the present disclosure provides a method for detecting a presence or an absence of minimal residual disease (MRD) in a subject, comprising: (a) assaying deoxyribonucleic acid (DNA) molecules from a first biological sample obtained or derived from said subject at a first time point; (b) detecting a set of biomarkers from said DNA molecules based at least in part on said assaying of (a), wherein said set of biomarkers comprise differentially expressed markers or variants; (c) generating a plurality of probe nucleic acids that are customized for said subject, wherein said probe nucleic acids comprises sequences of at least a subset of said set of biomarkers; (d) using said plurality of probe nucleic acids, sequencing cell free deoxynucleic acids (cfDNA) from a second biological sample obtained or derived from said subject at a second time point to detect the presence or absence of said subset of said set of biomarkers, wherein said sequencing is performed at a depth of at least 80×; (e) sequencing nucleic acids obtained or derived from said subject by using whole genome sequencing to determine a copy number of at least one region of a genome of a subject; and (f) computer processing said subset of said set of biomarkers and said copy number of said at least one region of a genome to detect said presence or absence of MRD in a subject.

In some embodiments, the first or second biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a saliva sample, tissue biopsy, pleural fluid sample, peritoneal fluid sample, amniotic fluid sample, cerebroshinal fluid sample, lymphatic fluid sample, sweat sample, tear sample, semen sample, or any derivative thereof, and any combination thereof. In some embodiments, the said first or second biological sample comprises said plasma sample. In some embodiments, wherein said first or second biological sample comprises said urine sample. In some embodiments, the first or second biological sample is obtained or derived from said subject using an ethylenediaminetetraacetic acid (EDTA) collection tube, a cell-free RNA collection tube, or a cell-free deoxyribonucleic acid (DNA) collection tube, other blood collection tube, and CTC collection tubes.

In some embodiments, (a) comprises subjecting said biological sample to conditions that are sufficient to isolate, enrich, or extract said DNA molecules. In some embodiments, the method further comprises fractionating said first biological sample of said subject to obtain said DNA molecules, wherein said first biological sample is a whole blood sample. In some embodiments, the method further comprises fractionating said second biological sample of said subject to obtain said cfDNA molecules, wherein said second biological sample is a whole blood sample.

In some embodiments, at least one of said DNA molecules are assayed using DNA sequencing to produce nucleic acid sequencing reads. In some embodiments, the DNA sequencing comprises whole exome sequencing. In some embodiments, the method further comprises filtering at least a subset of said nucleic acid sequencing reads based on a quality score. In some embodiments, the method further comprises performing error correction on said nucleic acid sequencing reads using sample barcodes or molecular barcodes attached to at least one of said cfDNA molecules. In some embodiments, the method further comprises performing at least one of single-stranded consensus calling and double-stranded consensus calling on said nucleic acid sequencing reads, thereby suppressing sequencing and PCR errors in said nucleic acid sequencing reads.

In some embodiments, the whole genome sequencing of (e) comprises low-pass whole genome sequencing. In some embodiments, the whole genome sequencing of (e) is performed at an average depth of no more than 2×.

In some embodiments, the sequencing of (d) is performed at a depth of at least 100×. In some embodiments, the sequencing of (d) is performed at a depth of at least 1,000×. In some embodiments, sequencing of (d) is performed at a depth of at least 10,000×. In some embodiments, sequencing of (d) is performed at a depth of at least 100,000×. In some embodiments, the sequencing of (e) comprises sequencing nucleic acids derived from said first biological sample. In some embodiments, the sequencing of (e) comprises sequencing nucleic acids derived from said second biological sample. In some embodiments, the sequencing of (e) comprises sequencing nucleic acids of a sample taken at said first time point, and sequencing nucleic acids of a sample taken at a second time point. In some embodiments, the method further comprises comparing the results of the sequencing of nucleic acids of said sample taken at said first time point and the sequencing of said nucleic acids of said sample taken at a second time point to determine said copy number of at least one region of a genome of a subject. In some embodiments, the method further comprises generating a baseline copy number at least based on said sequencing of said nucleic acids of said sample taken at said first time point.

In some embodiments, the assaying of (a), sequencing of (d), or sequencing of (e) comprises nucleic acid amplification. In some embodiments, the nucleic acid amplification comprises polymerase chain reaction (PCR) or isothermal amplification.

In some embodiments, the cancer is selected from the group consisting of: genitourinary cancer, breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. In some embodiments, the cancer comprises said bladder cancer. In some embodiments, the bladder cancer is a muscle invasive bladder cancer. In some embodiments, the subject is asymptomatic for said cancer.

In some embodiments, the method comprises detecting said presence or absence of minimal residual disease in said subject at an accuracy of at least about 60%, at least about 70%, at least about 75%, at least about 80%, at least about 85%, at least about 90%, at least about 95%, at least about 98%, or at least about 99%. In some embodiments, the method comprises detecting said presence or absence of minimal residual disease in said subject at a sensitivity of at least about 60%, at least about 70%, at least about 75%, at least about 80%, at least about 85%, at least about 90%, at least about 95%, at least about 98%, or at least about 99%. In some embodiments, the method comprises detecting said presence or absence of minimal residual disease in said subject at a specificity of at least about 60%, at least about 70%, at least about 75%, at least about 80%, at least about 85%, at least about 90%, at least about 95%, at least about 98%, or at least about 99%. In some embodiments, the method comprises detecting said presence or absence of minimal residual disease in said subject at a positive predictive value of at least about 60%, at least about 70%, at least about 75%, at least about 80%, at least about 85%, at least about 90%, at least about 95%, at least about 98%, or at least about 99%. In some embodiments, the method comprises detecting said presence or absence of minimal residual disease in said subject in said subject at a negative predictive value of at least about 60%, at least about 70%, at least about 75%, at least about 80%, at least about 85%, at least about 90%, at least about 95%, at least about 98%, or at least about 99%.

In some embodiments, the biological sample is obtained or derived from said subject prior to said subject receiving a therapy for said cancer. In some embodiments, the biological sample is obtained or derived from said subject during a therapy for said cancer. In some embodiments, the biological sample is obtained or derived from said subject after receiving a therapy for said cancer. In some embodiments, the therapy is selected from the group consisting of: surgical resection, chemotherapy, radiotherapy, immunotherapy, cell therapy, adjuvant therapy, neoadjuvant therapy, androgen deprivation therapy, and a combination thereof.

In some embodiments, the method further comprises identifying a clinical intervention for said subject based at least in part on said detected presence or said absence of said cancer. In some embodiments, the clinical intervention is selected from a plurality of clinical interventions.

In some embodiments, the clinical intervention is selected from the group consisting of: surgical resection, chemotherapy, radiotherapy, immunotherapy, adjuvant therapy, neoadjuvant therapy, androgen deprivation therapy, and a combination thereof. In some embodiments, the method further comprises administering said clinical intervention to said subject.

In some embodiments, the set of biomarkers comprises one or more members selected from the group consisting of genes listed in Table 1. In some embodiments, the set of biomarkers comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. In some embodiments, the set of biomarkers comprises one or more members selected from the group consisting of genes listed in Table 7. In some embodiments, the set of biomarkers comprises 2, 3, 4, 5, 6, 7, 8, 9, 10, 15, 20, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380,390, 400, 410, 420, 430, 440, 450, 460, 470, 480, 490, 500, 520, 540, 560, 580, 600, 700, 800, 900, or 1000 members selected from the group consisting of genes listed in Table 7. In some embodiments, the set of biomarkers comprises one or more members selected from the group consisting of genes listed in Table 8. In some embodiments, the set of biomarkers comprises 2, 3, 4, 5, 6, 7, 8, 9, 10, 15, 20, 25, 30, 35, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380,390, 400, 410, 420, 430, 440, 450, 460, 470, 480, 490, 500, 520, 540, 560, 580, 600, 700, 800, 900, or 1000 members selected from the group consisting of genes listed in Table 8. In some embodiments, the set of biomarkers comprises one or more members selected from the group consisting of genes listed in Table 9. In some embodiments, the set of biomarkers comprises 2, 3, 4, 5, 6, 7, 8, 9, 10, 15, 20, 25, 30, 35, 40, 45, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380,390, 400, 410, 420, 430, 440, 450, 460, 470, 480, 490, 500, 520, 540, 560, 580, 600, 700, 800, 900, or 1000 members selected from the group consisting of genes listed in Table 9. In some embodiments, the subset of said set of biomarkers comprises one or more members selected from the group consisting of genes listed in Table 9. In some embodiments, the subset of said set of biomarkers comprises 2, 3, 4, 5, 6, 7, 8, 9, 10, 15, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380,390, 400, 410, 420, 430, 440, 450, 460, 470, 480, 490, 500, 520, 540, 560, 580, 600, 700, 800, 900, or 1000 members selected from the group consisting of genes listed in Table 9.

In some embodiments, the plurality of probes comprises nucleic acid primers. In some embodiments, the plurality of probes comprises nucleic acid capture probes. In some embodiments, the plurality of probes comprises sequence complementarity with at least a portion of nucleic acid sequences of said set of biomarkers. In some embodiments, the plurality of probes comprises at least 2, 3, 4, 5, 6, 7, 8, 9, 10, 15, 20, 25, 30, 35, 40, 45, 50, 55, 60, 65, 70, 75, 80, 90, 95, 100, 105, 110, 115, 120, 125, 130, 135, 140, 145, 150, 155, 160, 165, 170, 175, or 180 different probes.

In some embodiments, (d) further comprises sequencing using a fixed plurality of probes wherein the probes of the fixed plurality of probes comprises probes that do not comprise sequences of said subset of said set of biomarkers. In some embodiments, the fixed plurality of probes comprise one or more members selected from the group consisting of genes listed in Table 10. In some embodiments, the method further comprises determining a likelihood of said determination of said presence or said absence of said cancer in said subject.

In some embodiments, the method further comprises monitoring said presence or said absence of said cancer in said subject, wherein said monitoring comprises assessing said presence or said absence of said cancer in said subject at each of a plurality of time points.

In some embodiments, a difference in said assessment of said presence or said absence of said cancer in said subject among said plurality of time points is indicative of one or more clinical indications selected from the group consisting of: (i) a diagnosis of said cancer, (ii) a prognosis of said cancer, and (iii) an efficacy or non-efficacy of a course of treatment for treating said cancer of said subject. In some embodiments, the prognosis comprises an expected progression-free survival (PFS) or overall survival (OS). In some embodiments, the set of biomarkers from said cfDNA molecules comprise tumor-associated alterations selected from the group consisting of: single nucleotide variants (SNVs), insertions or deletions (indels), and rearrangements.

In some embodiments, the method further comprises based at least on (e), detecting a copy number variation or copy number loss.

In some embodiments, the method further comprises determining, among said set of biomarkers, a mutant allele frequency of a set of somatic mutations. In some embodiments, the method further comprises determining a circulating tumor DNA (ctDNA) fraction of said cancer of said subject based at least in part on said set of mutant allele frequencies.

In some embodiments, the method further comprises determining a tumor mutational burden (TMB) of said cancer of said subject. In some embodiments, the method further comprises determining an abnormality score of said cancer of said subject based at least in part on said set of mutant allele frequencies.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows schematic regarding liquid biopsy assays across the cancer care timeline.

FIG. 2 shows an example workflow for a tissue agnostic, actionable MRD assay.

FIG. 3 shows example workflow for an MRD assay.

FIG. 4 shows the specificity of mutation detection in an example assay.

FIG. 5 shows an example workflow for generating a baseline profile.

FIG. 6 shows chart regarding the relationship between tumor fraction and MRD sensitivity.

FIG. 7 shows a chart of tumor mutation allele frequency (MAF) in clinical samples.

FIG. 8 shows a chart regarding analytical sensitivity of an example PredicineBEACON assay using different numbers of target mutations.

FIG. 9A-9H shows example study design, treatment evaluation, and biomarker investigation. FIG. 9A shows a scheme depicting the study design, sample collection, and biomarker analyses of the RJBLC-I2N003 trial. Patients underwent TURBT for tumor resection, pathologic diagnosis, disease staging, and risk stratification. All enrolled patients received preoperative toripalimab at 3 mg/kg every 2 weeks for up to 4 cycles. Imaging evaluation was performed at baseline and after every two treatment cycles. Radical cystectomy was planned within 4±2 weeks after the last dose of toripalimab treatment, after which surgical tissues were subjected to pathological evaluation and biomarker analysis. Urine and plasma samples were collected during the course of neoadjuvant immunotherapy. The PredicineBEACON™ MRD assay was performed to analyze utDNA and ctDNA.

FIG. 9B shows a Swimmer plot showing treatment course and clinical responses according to RECIST1.1. Reasons for early toripalimab termination are denoted in circled text. Reasons for surgical delay are indicated by text encircled by a box.

FIG. 9C shows a waterfall plot for the best change of target lesions in 20 patients. The best overall responses of each patient, according to RECIST1.1, are arranged along the x-axis. Bar color indicates the pathologic outcome of neoadjuvant toripalimab.

FIG. 9D shows a Sankey plot illustrating the pathologic outcome of neoadjuvant toripalimab. Tumor stages before therapy were assessed by MRI imaging, and tumor stages after therapy were evaluated by pathological examination.

FIG. 9E shows ROC curves for pre-treatment TFsm, TFcn, and MRI measurements in predicting ypCR, along with their corresponding AUC values.

FIG. 9F shows ROC curves for post-treatment TFsm, TFcn, and MRI measurements in predicting ypCR, along with their corresponding AUC values.

FIG. 9G shows a heatmap illustrating the relationship between pre- or post-treatment urinary MRD status and radiographic or pathologic outcome. Patients with utDNA clearance (defined by TFsm+TFcn<10%) or FGFR3 mutants following neoadjuvant toripalimab are also indicated.

FIG. 9H shows a proposed workflow for actionable utDNA MRD testing and clinical decision-making in MIBC patients receiving neoadjuvant therapy. Urine-based noninvasive MRD analysis enables adaptive management of individual patients to undergo either bladder preservation or radical cystectomy based on their real-time MRD status. TURBT=transurethral resection of bladder tumor; MRD=minimal residual disease; AE=adverse event; SAE=Serious adverse event; RECIST=Response Evaluation Criteria in Solid Tumors; CR=complete response; PR=partial response; SD=stable disease; PD=progressive disease; ypCR=pathological complete response; TFsm=tumor fraction estimate based on somatic mutations; TFcn=tumor fraction estimate based on copy numbers; MRI=magnetic resonance imaging; AUC=area under the receiver operating characteristic curve; utDNA=urinary tumor DNA; utDNA-pre=pre-treatment utDNA; utDNA-post=post-treatment utDNA.

FIG. 10A-B show representative images for evaluation of toripalimab. (A) Representative MRI images for radiographic evaluation of neoadjuvant toripalimab. (B) Representative hematoxylin and eosin staining for histopathological evaluation of neoadjuvant toripalimab. CR=complete response; PR=partial response; SD=stable disease; PD=progressive disease. A modifier “p” refers to pathologic staging after cystectomy.

FIG. 11A shows a stacked bar plot shows the percentage of patients with negative or positive PD-L1, low or high TMB, and negative or positive TLS. FIG. 11B shows a Oncoprint chart for the mutational landscape of tDNA in patients with ypCR or non-ypCR. Samples were analyzed by whole exome sequencing, and the mutation frequencies of each gene are shown on the right. FIG. 11C shows a line plot that illustrates tumor size changes measured by MRI imaging before and after neoadjuvant toripalimab. PD-L1=programmed death ligand 1; TMB=tumor mutation burden; TLS=tertiary lymphoid structure; ypCR=pathological complete response; pre-tx=pre-treatment; post-tx=post-treatment; MRI=magnetic resonance imaging.

FIG. 12A shows a Oncoprint chart for the mutational landscape of tDNA and utDNA. Samples were analyzed by whole exome sequencing, and the mutation frequencies of each gene are shown on the right. FIG. 12B shows TMB correlation between tDNA and utDNA as assessed by whole exome sequencing. Shading indicates 95% confidence interval. Spearman correlation coefficient (r) and P value are shown. FIG. 12C shows variant- and sample-level sensitivity across 93 titrated SeraCare reference samples. Each row presents a targeted mutation, and each column corresponds to a sample. An MRD positive event was called when the MRD score in a sample was no less than 2. FIG. 12D Variant- and sample-level specificity across urinary cell-free DNA samples from 16 healthy donors. Each row presents a targeted mutation, and each column corresponds to a sample. An MRD positive event was called when the MRD score in a sample was no less than 2. tDNA=tumor DNA; utDNA=urinary tumor DNA; TMB=tumor mutation burden; MRD=minimal residual disease; AA=amino acid; VAF=variant allele frequency

FIG. 13A shows a box plot that compares TFsm in utDNA versus ctDNA samples collected at baseline. FIG. 13B shows Venn plots that show the number of shared and unique variants in matched utDNA and ctDNA samples. FIG. 13C show box plot compares TFcn in utDNA versus ctDNA samples at baseline. FIG. 13D illustrates copy number gain and loss of utDNA and ctDNA, as identified by GISTIC2.0 TFsm=tumor fraction estimate based on somatic mutations; utDNA=urinary tumor DNA; ctDNA=circulating tumor DNA; TFcn=tumor fraction estimate based on copy numbers.

FIG. 14A shows a line plot that illustrates TFsm changes in utDNA samples upon neoadjuvant toripalimab. FIG. 14B shows a line plot that illustrates TFcn changes in utDNA samples before and after neoadjuvant toripalimab. FIG. 14C shows stacked bar plot that show the percentage of patients with low or high pre-treatment TFsm, TFcn, and MRI measurements according to the optimal cutoff points defined by ROC analysis. FIG. 14D shows stacked bar plot showed the percentage of patients with low or high post-treatment TFsm, TFcn, and MRI measurements according to the optimal cutoff points defined by ROC analysis. TFsm=tumor fraction estimate based on somatic mutations; TFcn=tumor fraction estimate based on copy numbers; MRI=magnetic resonance imaging; utDNA=urinary tumor DNA; utDNA-pre=pre-treatment utDNA; utDNA-post=post-treatment utDNA; ypCR=pathological complete response; pre-tx=pre-treatment; post-tx=post-treatment.

FIG. 15A shows a spider plot indicating dynamic changes of TFsm, TFcn, and MRI measurements for each patient during neoadjuvant toripalimab. FIG. 15B shows a box plot and illustrates utDNA clearance (defined by TFsm+TFcn<10%) in utDNA-pre versus utDNA-post samples. FIG. 15C shows an IGV plot showing the FGFR3 S249C mutation in patient RZ12 detected by MRD panel sequencing or whole exome sequencing. FIG. 15D shows VAF changes of the FGFR3 S249C mutation in patient RZ12 with progressive disease. TFsm=tumor fraction estimate based on somatic mutations; TFcn=tumor fraction estimate based on copy numbers; MRI=magnetic resonance imaging; C1=cycle 1; C2=cycle 2; C3=cycle 3; C4=cycle 4; RC=radical cystectomy; ypCR=pathological complete response; MRD=minimal residual disease; WES=whole exome sequencing; tDNA=tumor DNA; utDNA=urinary tumor DNA; utDNA-pre=pre-treatment utDNA; utDNA-post=post-treatment utDNA; C2D1=cycle 2 day 1; VAF=variant allele frequency; PD=progressive disease.

FIG. 16 shows a computer control system that is programmed or otherwise configured to implement methods provided herein.

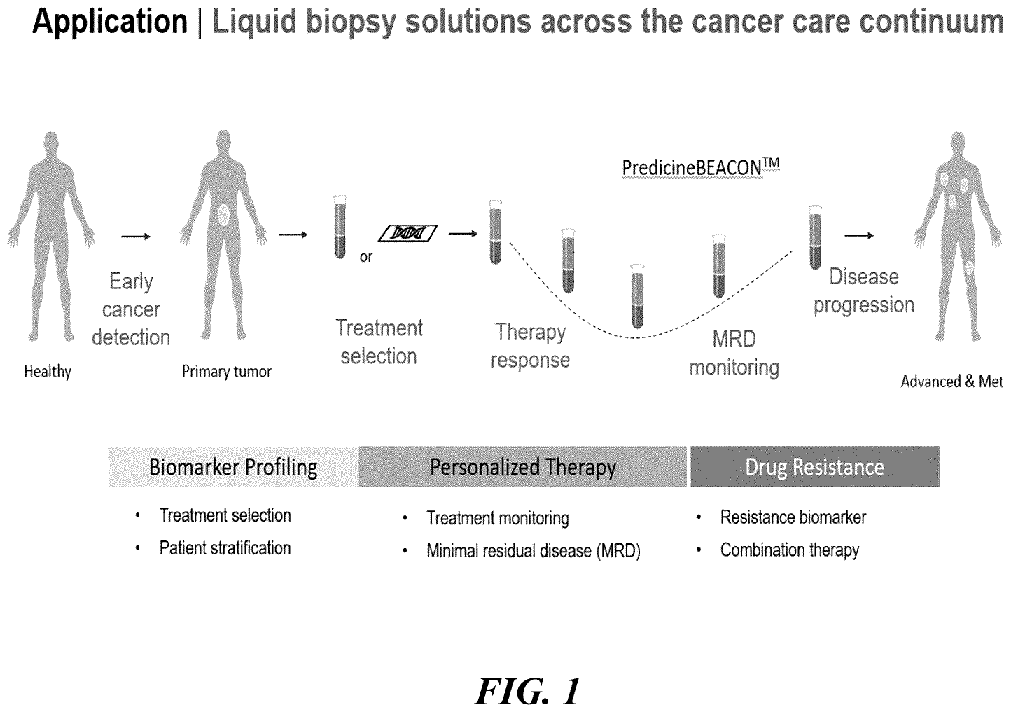

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods for detection of the presence or absence of cancer in a subject. The systems and methods provided herein comprises assaying polynucleotides to identify biomarkers of cancers in a subject. The biomarkers may be processed in order to identify the presence or absence of cancer. The methods described herein may process multiple type of analytes, or analytes from different sources or samples in order to determine a presence or absence of cancer. The multiple types of analytes may comprise DNA or RNA, for example cfDNA. The multiple analytes may be cfDNA, germline DNA, and cfRNA. By analyzing a plurality of different analytes or different sources or samples the methods may allow for improved detection or determination of a prognosis as compared to methods performed on fewer analytes or only one of many different analytes.

Circulating tumor DNA (ctDNA) can be used as a biomarker for noninvasive monitoring of treatment response and disease progression in many patients with cancer. Longitudinal and personalized ctDNA detection for monitoring treatment efficacy and minimal residual disease (MRD) can be used for detecting relapse early and assessing treatment responses. However, most current methods lack adequate sensitivity and actionable variant calling for residual disease detection during or after completion of treatment in non-metastatic cancer patients. To achieve high sensitivity for actionable MRD detection, a method can be using ctDNA analysis with high coverage sequencing and integrating results from multiple mutations in each patient.

A patient-specific, custom built liquid biopsy NGS assay for detecting MRD, and monitoring treatment response or recurrence can be performed by using cell free DNA (cfDNA) from blood or urine. The assay can 1) establish a baseline using either tissue, blood or urine samples, to detect genome-wide ctDNA variants (e.g. Single Nucelotide Variants (SNV)/Indels, fusions and copy number variants (CNV); and 2) monitor ctDNA variants using personalized mutation probes, a set of fixed core probes with actionable genes, and assay genome-wide CNV measurement.

The assay can identify somatic mutations in the subject. Somatic mutations can be identified by using a sequencing assay. Somatic mutations can be identified by using whole exome sequencing. The whole exome sequencing can comprise sequencing across at least genes. Additionally, the sequencing can be boosted, such that a higher depth is achieved, at specific exon or genes of interest, for example, cancer-related genes. For example, at least 100, 200, 300, 400, 500, 600, 700, 800, 900, or more genes can be sequenced at a higher depth than the rest of the exome (e.g. average depth of the whole exome sequencing).

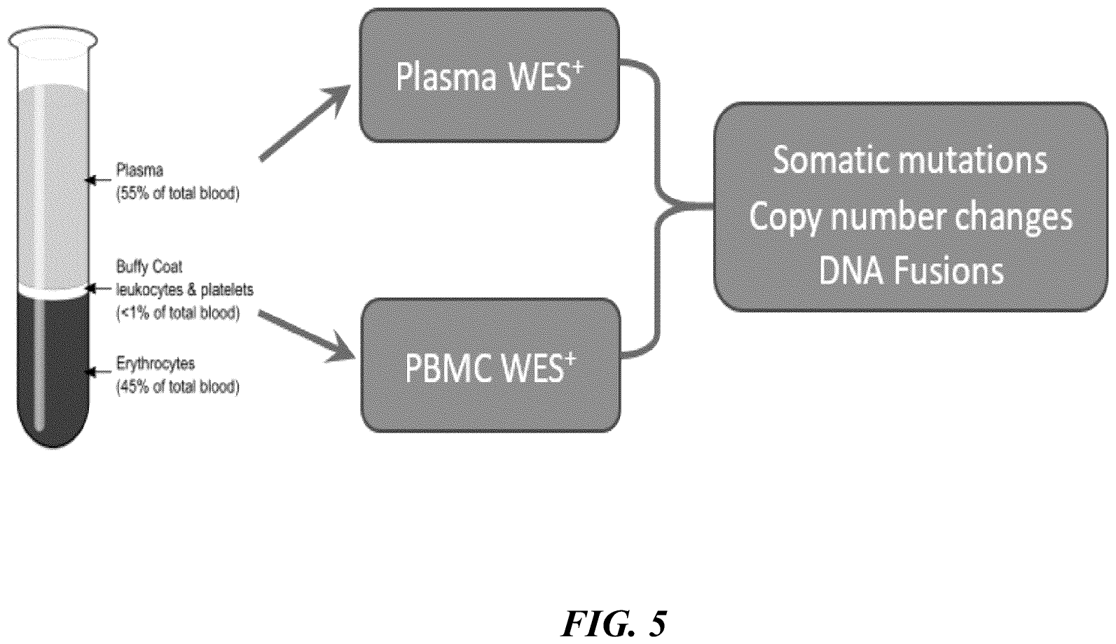

To detect reliable somatic mutations or fusions for personalized probe design, a matched control sample can be used. The matched control can be compared such to identify mutations or other variants. For example, a peripheral blood mononuclear cell (PBMC) normal control sample can be used. The PBMC sample can help to remove Clonal Hematopoiesis of Indeterminite Potential (CHIP) mutations, germline variants and other background variants. FIG. 5 shows an example workflow of obtaining a match PMBC sample. A whole blood sample of a subject can be taken and then separated into a plasma fraction and a PBMC sample. These two samples can be analyzed and compared to identify somatic mutations or fusions.

Once somatic mutations are detected and identified in a subject, a set of personalized or customized probes to mutations can be chosen for a subject. For example, 16-50 personalized somatic mutations or fusions can be selected for a given patient. In addition to this personalized panel, a fixed MRD core panel can be utilized for MRD detection and monitoring treatment efficacy. In this manner, genes that are relevant for a particular type of cancer or are otherwise known to be related to cancer can be analyzed, while simultaneously assaying for genes that are specific to a given subjects cancer. The fixed MRD core panel enables detecting novel and actionable mutations beyond variants identified in the baseline, which is critical for treatment monitoring, studying drug resistance, and guiding personalized therapies. Based at least upon this multi-layer panel design, the mutant allele fraction (MAF) limit of detection (LOD) for these assays can reach 0.005%.

Once a panel has been constructed, the subject can be monitored by obtaining samples from a subject and assaying the sample using the panel. Sequencing can be used to monitor the subject. Increasing the depth of this sequencing reaction can improve the detection sensitivity. For example, ultra-deep sequencing (e.g., 100,000×) can be performed and allow for high detection sensitivity.

In addition to the high detection sensitivity of the mutations, a companion whole genome sequencing (WGS) can be performed to monitor CNV changes for both baseline and follow up timepoints. The whole genome sequencing can be a low pass whole genome sequencing (LP-WGS; e.g., 1×). This can allow for more economical sequencing that can identify CNVs, and SNVs (or other genetic variants and mutations) at a high accuracy and sensitivity.

Tumor tissue is used for baseline profiling by most MRD assays. However, in many cases tumor tissues are not available, or tissue quality does not meet the assay requirements. Therefore, there is a significant clinical need to establish an MRD mutation baseline without the requirement of a tissue sample.

The subject may be a suspected of a suffering from a cancer. The cancer may be specific or originating from an organ or other area of the subject. For example, the cancer may be breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. The cancer may be a hormone sensitive prostate cancer (HSPC), castrate-resistant prostate cancer (CRPC), metastatic prostate cancer, and a combination thereof. The cancer may be muscle invasive bladder cancer (MIBC). The cancer may comprise biomarkers that are specific to a particular cancer. The specific biomarkers may indicate a presence of a particular cancer. For example, biomarker may indicate that a castrate-resistant prostate cancer is present. The biomarker may indicate that a MIBC is present. The identification of the presence of a type of cancer may allow the determination of a treatment option or recommendation.

In some cases, the subject may be asymptomatic for cancer. For example, the cancer may not exhibit any symptoms and the subject may be unaware of the presence of cancer. The methods described herein may allow a cancer to be identified at an earlier stage than otherwise. The identification of the presence of the cancer at an earlier stage may allow a treatment option or recommendation to be determined at an earlier stage and may allow the subject to have an improved prognosis. The subject may have had cancer and no longer shows symptoms of cancer. The identification of the presence of the cancer at an earlier stage or the recurrence or relapse of cancer may allow the subject to have an improved prognosis. The identification of the presence of the cancer may allow for a determination of a efficacy of a treatment.

The biological sample may comprise nucleic acids. The biological sample be a cell-free deoxyribonucleic acid (cfDNA) sample or a cell-free ribonucleic acid (cfRNA) sample. The biological sample may comprise genomic DNA or germline DNA(gDNA). The nucleic acid may be a DNA (e.g. double-stranded DNA, single-stranded DNA, single-stranded DNA hairpins, cDNA, genomic DNA, germline DNA, circulating tumor DNA (ctDNA), cell-free DNA (cfDNA)), an RNA (e.g. cfRNA, mRNA, cRNA, miRNA, siRNA, miRNA, snoRNA, piRNA, tiRNA, snRNA), or a DNA/RNA hybrids. The biological sample may be a derived from or contain a biological fluid. For example, the biological sample may be a plasma sample, a serum sample, a buffy coat sample, a peripheral blood mononuclear cell (PBMC) sample, a red blood cell sample, a urine sample, a saliva sample, or other body fluid sample. The biological sample may comprise or be a pleural fluid sample, peritoneal fluid sample, amniotic fluid sample, cerebrospinal fluid sample, lymphatic fluid sample, sweat sample, tear sample, semen sample, or any combination of biological fluid. In some case, the samples may comprise RNA and DNA. For example, a sample may comprise cfDNA and cfRNA.

The biological sample may be collected, obtained, or derived from said subject using a collection tube. The collection tube may be an ethylenediaminetetraacetic acid (EDTA) collection tube, a cell-free RNA collection tube, or a cell-free deoxyribonucleic acid (DNA) collection tube and CTC collection tubes, or other blood collection tube. The collection tube may comprise additional reagents for stabilizing the nucleic acid molecules or blood cells. The collection tube may allow the nucleic acid or blood cells to be stable such to minimize degradation of the biological sample prior to assaying. The additional reagents may comprise buffer salts or chelators.

The biological sample may be obtained or derived from a subject at a various times. The biological sample may be obtained or derived from a subject prior to the subject receiving a therapy for cancer. The biological sample may be obtained or derived from a subject during receiving a therapy for cancer. The biological sample may be obtained or derived from a subject after receiving a therapy for cancer. The biological sample may be collected over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

In various aspects as described herein, a clinical intervention or a therapy may be identified at least in part based on the identification of the presences of cancer, or the presence of a parameter of cancer. The clinical intervention may be a plurality of clinical interventions. The clinical intervention may be selected from a plurality of clinical interventions. The clinical intervention may be a surgical resection, chemotherapy, radiotherapy, immunotherapy, adjuvant therapy, neoadjuvant therapy, androgen deprivation therapy, or a combination thereof. In some cases, the clinical interventions may be administered to the subject. After administration of the clinical intervention, a sample may be obtained or derived from the subject such to monitor the cancer or cancer parameters. As such, the methods and systems disclosed herein may be performed iteratively such that monitoring of a cancer can be performed. Additionally, by performing the methods or systems iteratively, therapies or clinical interventions may be updated based on the results of the methods. The monitoring of the cancer may include an assessment as well as a difference in assessment from a previously generated assessment. The difference in an assessment of cancer in said subject among a plurality of time points (or samples) may be indicative of one or more clinical indications such as a diagnosis of said cancer, a prognosis of said cancer, or an efficacy or non-efficacy of a course of treatment for treating said cancer of said subject. The prognosis may comprise expected progression-free survival (PFS), overall survival (OS), or other metrics relating the severity or survivability of a cancer.

The biological samples may be subjected to additional reactions or conditions prior to assaying. For example, the biological sample may be subjected to conditions that are sufficient to isolate, enrich, or extract nucleic acids, such cfDNA molecules or cfRNA molecules.

The methods disclosed herein may comprise conducting one or more enrichment reactions on one or more nucleic acid molecules in a sample. The enrichment reactions may comprise contacting a sample with one or more beads or bead sets. The enrichment reactions may comprise one or more hybridization reactions. For example, the enrichment reactions may comprise contacting a sample with one or more probes (e.g., capture probes) or bait molecules that hybridize to a nucleic acid molecule of the biological sample. The enrichment reaction may comprise differential amplification of a set of nucleic acid molecules. The enrichment reaction may enrich for a plurality of genetic loci or sequences corresponding to genetic loci. For example, the enrichment reaction may enrich for sequences corresponding to genes from Table 1, Table 7, Table 8, Table 9, or Table 10. The enrichment reactions may comprise the use of primers or probes that may complementarity to sequences (or sequences upstream or downstream) of a sequence that is to be enriched. For example, a capture probe may comprise sequence complementarity to a set of genomic loci and allow the enrichment of the genomic loci. The enrichments reactions may comprise a plurality of probes or primers. A plurality of probes may comprise 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes. The probes can be a biotinylated probe. The probes can be attached to a bead or other solid support. The probes can be attached to a bead or other solid support via a non-covalent (e.g., biotin-streptavidin interaction) or a covalent interaction. The solid support can be a magnetic solid support.

The methods disclosed herein may comprise conducting one or more isolation or purification reactions on one or more nucleic acid molecules in a sample. The isolation or purification reactions may comprise contacting a sample with one or more beads or bead sets. The isolation or purification reaction may comprise one or more hybridization reactions, enrichment reactions, amplification reactions, sequencing reactions, or a combination thereof. The isolation or purification reaction may comprise the use of one or more separators. The one or more separators may comprise a magnetic separator. The isolation or purification reaction may comprise separating bead bound nucleic acid molecules from bead free nucleic acid molecules. The isolation or purification reaction may comprise separating capture probe hybridized nucleic acid molecules from capture probe free nucleic acid molecules. The isolation reactions may comprises removing or separating a group of nucleic acid molecules from another group of nucleic acids.

The methods disclosed herein may comprise conduction extraction reactions on one or more nucleic acids in a biological sample. The extraction reactions may lyse cells or disrupt nucleic acid interactions with the cell such that the nucleic acids may be isolated, purified, enriched or subjected to other reactions.

The methods disclosed herein may comprise amplification or extension reactions. The amplification reactions may comprise polymerase chain reaction. The amplification reaction may comprise PCR-based amplifications, non-PCR based amplifications, or a combination thereof. The one or more PCR-based amplifications may comprise PCR, qPCR, nested PCR, linear amplification, or a combination thereof. The one or more non-PCR based amplifications may comprise multiple displacement amplification (MDA), transcription-mediated amplification (TMA), nucleic acid sequence-based amplification (NASBA), strand displacement amplification (SDA), real-time SDA, rolling circle amplification, circle-to-circle amplification or a combination thereof. The amplification reactions may comprise an isothermal amplification.

The method disclosed herein may comprise a barcoding reaction. A barcoding reaction may comprise the additional of a barcode or tag to the nucleic acid. The barcode may be a molecular barcode or a sample barcode. For example, a barcode nucleic acid may comprise a barcode sequence which may be a degenerate n-mer. The sequence may be randomly generated or generated such to synthesize a specific barcode sequence. The barcode nucleic acid may be added to a sample such to label the nucleic acid molecules in the sample. The barcodes may be specific to a sample. For example, a plurality of barcode nucleic acids may be added to a sample in which the barcode sequence is the same. Upon barcoding of the nucleic acids, those originating from a same sample may have a same barcode sequence, and may allow a nucleic acid to be identified as belonging to a particular or given sample. A molecular barcode may also be used such that each molecule (or a plurality of molecules) in a same volume have a different molecular barcode. This barcode may be subjected to amplification such that all amplicons derived from a molecule have the same barcode. In this way, molecules originating from a same molecule may be identified. The sequences reads may be processed based on the barcode sequences. For example, the processing may reduce errors or allow a molecule to be tracked. Barcode sequences may be appended or otherwise added or incorporated into a sequence by various reactions, for example an amplification, extension, or ligation reaction, and may be performed enzymatically using a nucleic acid polymerase or ligase. The ligation may be an overhang or blunt end ligation and the barcodes may comprise complementarity to nucleic acids to be barcoded. This complementarity may be a sequence derived from the sample from the subject or may be constant sequence generated via a reaction performed on the nucleic acids in the sample.

In some cases, the biological sample may comprise multiple components. For example, the biological sample may be a whole blood sample. The biological sample may be subjected to reactions such to separate or fractionate a biological sample. For example, a whole blood sample may be a fractionated and cell free nucleic acids may be obtained. The whole blood sample may be fractionated using centrifugation such that blood cells may be separated from the plasma (which may contain cell free nucleic acid). A sample may be subjected to multiple rounds of separation or fractionation.

A given biological sample may be subjected to multiple different reactions. For example a given biological sample may be subjected to multiple different sequencing reactions. For example, the sample may be subjected to a whole exome sequencing reaction and a whole genome sequencing. The sample may be divided into multiple samples, and one part of the sample may be subjected to reaction, and another part may be subjected to another reaction.

For example, a biological sample may be split or otherwise divided to form multiple samples. The resulting sample may comprise same compositions. The biological samples may be fractionated, for example, into plasma and erythrocyte fraction.

In various aspects described throughout the disclosure, the nucleic acids may be subjected to sequencing reactions. The sequencing the reactions may be used on DNA, RNA or other nucleic acid molecules. Example of a sequencing reaction that may be used include capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. The sequencing reactions may comprise whole genome sequencing, whole exome sequencing, low-pass whole genome sequencing, targeted sequencing, methylation-aware sequencing, enzymatic methylation sequencing, bisulfite methylation sequencing. The sequencing reaction may be a transcriptome sequencing, mRNA-seq, totalRNA-seq, smallRNA-seq, exosome sequencing, or combinations thereof. Combinations of sequencing reactions may be used in the methods described elsewhere herein. For example, a sample may be subjected to whole genome sequencing and whole transcriptome sequencing. As the samples may comprise multiple types of nucleic acids (e.g. RNA and DNA), sequencing reactions specific to DNA or RNA may be used such to obtain sequence reads relating to the nucleic acid type.

The sequencing reactions can be performed at various sequencing depths. The sequencing depths of a sequencing reaction may be selected or modulated. The sequencing reactions may comprise sequencing at a region a depth of at least 1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 60×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 70,000×, 80,000×, 90,000, 100,000×, or more. The sequencing reactions may comprise sequencing a region at a depth of no more than 1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 60,000×, 70,000×, 80,000×, 100,000×, or less.

In various embodiments, a low pass whole genome sequencing is used to sequence nucleic acids. The low pass whole genome sequence may be performed at an average sequencing depth of at least 1×, 2×, 3×, 4×, 5×, 6×, 7×, 8×, 9×, 10×, or more. The low pass whole genome sequence may be performed at an average sequencing depth of no more than 1×, 2×, 3×, 4×, 5×, 6×, 7×, 8×, 9×, 10×, or less. The low pass whole genome sequencing may be performed at an average depth of between 1× and 2×.

In various embodiments, a sequencing reaction may be performed using a set of personalized or customized probes. The sequencing reaction using a set of personalized or customized probes may be a deep sequencing reaction or ultra-deep sequencing reaction. For example, the sequencing reaction using a set of personalized or customized probes may be performed at an sequencing depth of 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 20,000×, 30,000×, 40,000×, 50,000×, 60,000×, 70,000×, 80,000×, 90,000, 100,000×, or more.

In various embodiments, a whole exome sequencing is used to sequence nucleic acids of a subject. The whole exome sequencing may be performed at a non-uniform depth. For example, certain areas of the exome may be boosted or otherwise sequenced at a greater depth than other regions, or at a greater depth than the average depth of the whole exome sequencing. By sequencing certain regions at a higher depth, genes or regions that are of more interest may be analyzed with higher sensitivity, accuracy, and/or precision. Genes or regions associated with or related to cancer can be sequenced at a greater depth. For example, at least 100, 200, 300, 400, 500, 600, 700, 800, 900, or more genes can be sequenced at a higher depth than the rest of the exome (e.g. average depth of the whole exome sequencing).

The sequencing of nucleic acids may generate sequencing read data. The sequencing reads may be processed such to generate data of improved quality. The sequencing reads may be generated with a quality score. The quality score may indicate an accuracy of a sequence read or a level or signal above a nose threshold for a given base call. The quality scores may be used for filtering sequencing reads. For example, sequencing reads may be removed that do not meet a particular quality score threshold. The sequencing reads may be processed such to generate a consensus sequence or consensus base call. A given nucleic acid (or nucleic acid fragment) may be sequenced and errors in the sequence may be generated due to reactions prior or during sequencing. For example, amplification or PCR may generate error in amplicons such that the sequences are not identical to a parent sequence. Using sample barcodes or molecular barcodes, error correction may be performed. Error correction may include identifying sequence reads that do not corroborate with other sequences from a same sample or same original parent molecules. The use of barcodes may allow the identification or a same parent or sample. Additionally, the sequence reads may be processed by performing single strand consensus calling or double stranded consensus call, thereby reducing or suppressing error.

The methods as disclosed herein may comprise determining allele frequency or other cancer related metric. The methods may comprise a mutant allele frequency of a set of somatic mutation among a set of biomarkers. The mutant allele frequency may be used to determine a circulating tumor DNA (ctDNA) fraction of a cancer of a subject. A plasma tumor mutational burden (pTMB) of a cancer of the subject may be determined based at least in part on the set of mutant allele frequencies. Detection of microsatellite instability may also be used to determine the presence or absence of a cancer or cancer metric. Methylation states may be determined using methods described herein and may be used to identify a presence of a cancer or cancer parameter.

In various aspects, sets of biomarkers are processed and data corresponding to the biomarkers are generated. The sets of biomarkers may comprise quantitative or qualitative measures from a set of genomic loci. The set of genomic loci may comprise a set of cancer-associated genomic loci. The sets of biomarkers may correspond to a set of genes. The sets of biomarkers may comprise one or more genes selected from Table 1. In some case, a set of biomarkers may comprise 2, 3, 4, 5, 6, 7, 8, 9, 10, 15, 20, 25, 30, 35, 40, 45, 50, 55, 60, 65, 70, 80, 85, 90, 95, 100, 105, 110, 115, 120, 125, 130, 135, 140, 145, 150, 155, 160, 165, 170, 175, or 180 members selected from the group consisting of genes listed in Table 1.

TABLE 1
List of genes

| | | | | | | | |
|---|---|---|---|---|---|---|---|
| ABRAXAS1 | AKT1 | ALK | APC | AR | ARAF | ARID1A | ATM |
| ATR | BAP1 | BARD1 | BRAF | BRCA1 | BRCA2 | BRIP1 | CCND1 |
| CCNE1 | CD274 | CDH1 | CDK12 | CDK4 | CDK6 | CDKN2A | CHEK1 |
| CHEK2 | CTNNB1 | DDR2 | DNAJB1 | EGFR | EPCAM | ERBB2 | ERBB3 |
| ERCC1 | ERCC2 | ERCC4 | ESR1 | EZH2 | FANCA | FANCC | FANCD2 |
| FANCI | FANCL | FANCM | FBXW7 | FGFR1 | FGFR2 | FGFR3 | GNA11 |
| GNAQ | GNAS | HDAC2 | HRAS | IDH1 | IDH2 | JAK2 | JAK3 |
| KIT | KRAS | MAP2K1 | MAP2K2 | MAPK1 | MDM2 | MET | MLH1 |
| MPL | MRE11 | MSH2 | MSH6 | MTOR | MYC | MYCN | MYD88 |
| NBN | NF1 | NFE2L2 | NPM1 | NRAS | NTRK1 | NTRK3 | PALB2 |
| PDCD1LG2 | PDGFRA | PIK3CA | PMS2 | POLD1 | POLE | PPM1D | PPP2R1A |
| PPP2R2A | PRKACA | PRKD1 | PTEN | PTPN11 | RAD50 | RAD51 | RAD51B |
| RAD51C | RAD51D | RAD54L | RAF1 | RB1 | RECQL | RET | RNF43 |
| ROS1 | RPA1 | SMAD4 | SMO | SPOP | STK11 | TERT | TMPRSS2 |
| TP53 | TP53BP1 | TSC1 | TSC2 | VHL | XRCC2 | XRCC3 | XRCC4 |

The sets of biomarkers may comprise one or more genes selected from Table 7. In some case, a set of biomarkers may comprise 2, 3, 4, 5, 6, 7, 8, 9, 10, 15, 20, 25, 30, 35, 40, 45, 50, 55, 60, 65, 75, 80, 85, 90, 95, 100, 105, 110, 115, 120, 125, 130, 135, 140, 145, 150, 155, 160, 165, 170, 175, or 180 members selected from the group consisting of genes listed in Table 7.

The sets of biomarkers may comprise one or more genes selected from Table 8. In some case, a set of biomarkers may comprise 2, 3, 4, 5, 6, 7, 8, 9, 10, 15, 20, 25, 30, 35, 40, 45, 50, 55, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 8. The sets of biomarkers may comprise one or more genes selected from Table 9. In some case, a set of biomarkers may comprise 2, 3, 4, 5, 6, 7, 8, 9, 10, 15, 20, 25, 30, 35, 40, 45, 50, 55, 60, 65, 75, 80, 85, 90, 95, 100, 105, 110, 115, 120, 125, 130, 135, 140, 145, 150, 155, 160, 165, 170, 175, or 180 members selected from the group consisting of genes listed in Table 9. The sets of biomarkers may comprise one or more genes selected from Table 10. In some case, a set of biomarkers may comprise 2, 3, 4, 5, 6, 7, 8, 9, 10, 15, 20, 25, 30, 35, 40, 45, 50, 55, 60, 65, 70, 80, 85, 90, 95, 100, 105, 110, 115, 120, 125, 130, 135, 140, 145, 150, 155, 160, 165, 170, 175, or 180 members selected from the group consisting of genes listed in Table 10.

The sets of biomarkers may correspond to genetic aberration of a genetic locus. The genetic aberration may a tumor associated alteration. The genetic aberration may be a copy number alterations (CNAs), copy number losses (CNLs), single nucleotide variants (SNVs), insertions or deletions (indels), and rearrangements. The set of biomarkers may be identified in a variety of nucleic acid types. For example, the tumor associated alteration may be identified in cfDNA. The tumor associated alteration may comprise changes in allelic expression, or gene expression. Methods and systems disclosed herein may allow for gene expression profiling and identification of changes to the expression levels of gene.

In various aspects, the methods may comprise identifying the presence of a copy number or a copy number variation. The method may comprise using a whole genome sequencing reactions to identify a copy number of a gene or region. The method may comprise identifying a copy number of a gene or region at a first time point or in a first sample or part of a sample. The copy number identified may be used a baseline. The method may comprise identifying a copy number of a gene or region at a different time point, sample or part of a sample and may be used to compare to a baseline. By comparing to a baseline, the method may allow identification of change in copy number over time, or a difference in copy number between two samples.

In various aspects, a baseline measurement of a parameter is generated. A sample taken at a first time point may be used to compare to samples taken at a second time point. Deviations from a baseline sample can indicate the presence or absence of a genetic aberration I in a subject. For example, an increase in a copy number as compared to baseline may be used to assess the presence of a copy number increase. In another example, a baseline can comprise mutations and the mutations can be identified in sample taken at another time. This presence of the mutations in a later sample may indicated that a cancer is present in the subject.

In various aspects, the methods may comprise identifying the presence of a cancer or a cancer parameter. The method may process multiple data sets to identify a presence of cancer or cancer parameter. For example, the methods may comprise using data derived from a whole genome reaction and a targeted sequencing reaction. The methods may comprises determining a probability or a likelihood of the presence of cancer or a cancer parameter. For example, instead of a binary output indicating a presence or absence, an output may be generated that indicates a probability that subject has cancer. This probability may be determined based on algorithms as described elsewhere herein. Similarly, a probability or likely of response to a particular treatment or a probability of relapse may be outputted.

In various aspects, the sets of biomarkers are processed using an algorithm. The algorithm may be a trained algorithm. The trained algorithms may use the sets of biomarkers as an input and generate an output regarding the presence or absence of a cancer. The output may be specific to a type of cancer or subtype of cancer. For example, the output may indicate the presence of a muscle invasive bladder cancer.

The trained algorithm may be trained on multiple samples. For example, the trained algorithm may be trained using at least 2, 3, 4, 5, 6, 7, 8, 9, 10, 15, 20, 25, 30, 35, 40, 45, 50, 55, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or more independent training samples. The trained algorithm may be trained using no more 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or less, independent training samples. The training samples may be associated with a presence or an absence of said cancer. The training samples may be associated with a relapse of cancer. The training samples may be associated with cancer that is resistant to a particular drug or treatment. An individual training sample may be positive for a particular cancer. An individual training sample may be negative for a particular cancer. By using training samples, the trained algorithm may be able to detect a cancer, determine a probability of recurrence or relapse of a cancer, or determine if a cancer comprises a set of biomarkers may be resistant to a treatment. The training sample may be associated with additional clinical health data of a subject. For example, additional clinical health data may comprise the gender, weight, height, or levels of metabolites or antibodies in a subjects. Additional clinical health data may comprise indication of other diseases, disorders, or diseases conditions.

The trained algorithms may be trained using multiple sets of training samples. The sets may comprise training samples as described elsewhere herein. For example, the training may be performed using a first set of independent training samples associated with a presence of said cancer and a second set of independent training samples associated with an absence of said cancer. Similarly, a first set may be associated with relapse and a second sample may be associated with the absence of relapse.

The trained algorithm may also process additional clinical health data of the subject. For example, additional clinical health data may comprise the gender, weight, height, or levels of metabolites or antibodies in a subject. Additional clinical health data may comprise indication of other diseases, disorders, or diseases conditions that the subject may suffer from. By using the additional clinical health data, in conjunction with the biomarkers, the trained algorithm may output a presence or absences of cancer, probability of relapse, or resistance to drug treatment, that may be different from the output of an algorithm that does not process additional clinical health.

The trained algorithm may be an unsupervised machine learning algorithm. For example, the unsupervised machine learning algorithm may utilize cluster analysis to identify attributes of interest. The trained algorithm may be a supervised machine learning algorithm. For example, the algorithm may be inputted with training data such to generate an expected or desired output. The supervised learning algorithm may comprise a deep learning algorithm, a support vector machine (SVM), a neural network, or a Random Forest. Via the machine learning algorithm, the trained algorithm may be able to identify relationships of biomarkers to a particular cancer prognosis or diagnosis. Without the trained algorithm, it may otherwise be difficult to identify relationships of the biomarkers to accurately identify the presence of a cancer or other parameters associated with the cancer.

In various aspects, the systems and methods may comprise an accuracy, sensitivity, or specificity of detection of the cancer or a parameter of the cancer. For example, the methods or systems may comprise detecting the presence or the absence of cancer (or the presence of a parameter of the cancer, such as recurrence, relapse, or drug resistance) in the subject at an accuracy of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a sensitivity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a specificity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a positive predictive value of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a negative predictive value of at least about 60%, at least about 70%, at least about 75%, at least about 80%, at least about 85%, at least about 90%, at least about 95%, at least about 98%, or at least about 99%.

### Computer Control Systems

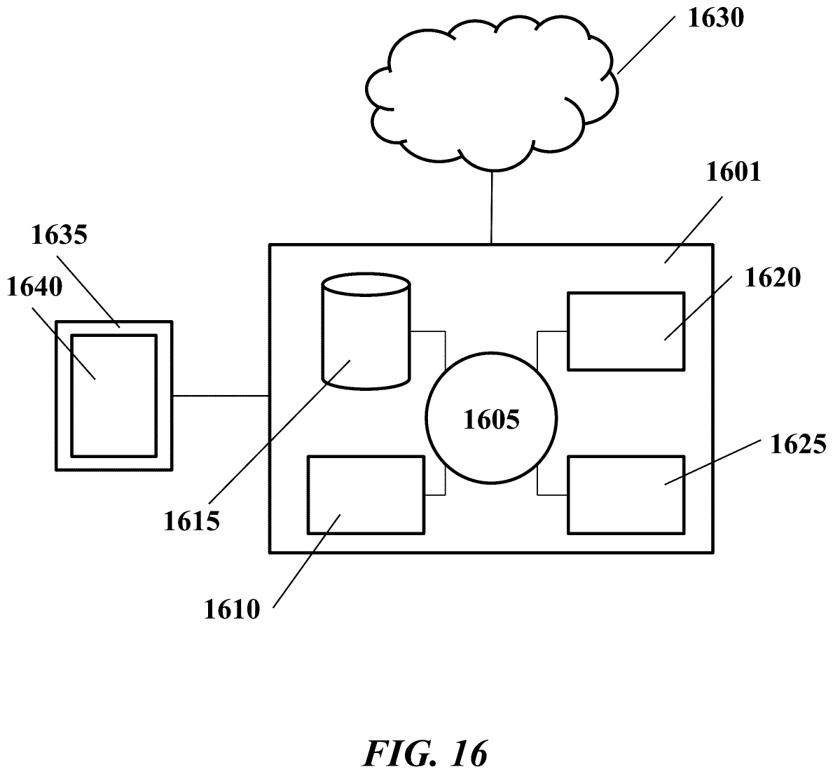

The present disclosure provides computer systems that are programmed to implement methods of the disclosure. FIG. 16 shows a computer system 1601 that is programmed or otherwise configured to perform analysis or steps of the methods, for example determine a likelihood of the presence of a cancer based on a set of biomarkers of an individual or run an algorithm. The computer system 1601 can regulate various aspects of methods and systems of the present disclosure, such as, for example, perform an algorithm, input training data, analyze sets of biomarker, or output a result for the user as to the presence or absence of cancer. The computer system 1601 can be an electronic device of a user or a computer system that is remotely located with respect to the electronic device. The electronic device can be a mobile electronic device.

The computer system 1601 includes a central processing unit (CPU, also “processor” and “computer processor” herein) 1605, which can be a single core or multi core processor, or a plurality of processors for parallel processing. The computer system 1601 also includes memory or memory location 1610 (e.g., random-access memory, read-only memory, flash memory), electronic storage unit 1615 (e.g., hard disk), communication interface 1620 (e.g., network adapter) for communicating with one or more other systems, and peripheral devices 1625, such as cache, other memory, data storage and/or electronic display adapters. The memory 1610, storage unit 1615, interface 1620 and peripheral devices 1625 are in communication with the CPU 1605 through a communication bus (solid lines), such as a motherboard. The storage unit 1615 can be a data storage unit (or data repository) for storing data. The computer system 1601 can be operatively coupled to a computer network (“network”) 1630 with the aid of the communication interface 1620. The network 1630 can be the Internet, an internet and/or extranet, or an intranet and/or extranet that is in communication with the Internet. The network 1630 in some cases is a telecommunication and/or data network. The network 1630 can include one or more computer servers, which can enable distributed computing, such as cloud computing. The network 1630, in some cases with the aid of the computer system 1601, can implement a peer-to-peer network, which may enable devices coupled to the computer system 1601 to behave as a client or a server.

The CPU 1605 can execute a sequence of machine-readable instructions, which can be embodied in a program or software. The instructions may be stored in a memory location, such as the memory 1610. The instructions can be directed to the CPU 1605, which can subsequently program or otherwise configure the CPU 1605 to implement methods of the present disclosure. Examples of operations performed by the CPU 1605 can include fetch, decode, execute, and writeback.

The CPU 1605 can be part of a circuit, such as an integrated circuit. One or more other components of the system 1601 can be included in the circuit. In some cases, the circuit is an application specific integrated circuit (ASIC).

The storage unit 1615 can store files, such as drivers, libraries and saved programs. The storage unit 1615 can store user data, e.g., user preferences and user programs. The computer system 1601 in some cases can include one or more additional data storage units that are external to the computer system 1601, such as located on a remote server that is in communication with the computer system 1601 through an intranet or the Internet.

The computer system 1601 can communicate with one or more remote computer systems through the network 1630. For instance, the computer system 1601 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1601 via the network 1630.

Methods as described herein can be implemented by way of machine (e.g., computer processor) executable code stored on an electronic storage location of the computer system 1601, such as, for example, on the memory 1610 or electronic storage unit 1615. The machine executable or machine readable code can be provided in the form of software. During use, the code can be executed by the processor 1605. In some cases, the code can be retrieved from the storage unit 1615 and stored on the memory 1610 for ready access by the processor 1605. In some situations, the electronic storage unit 1615 can be precluded, and machine-executable instructions are stored on memory 1610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1601 can include or be in communication with an electronic display 1635 that comprises a user interface (UI) 1640 for providing, for example, an input of biomarkers or sequencing data, or an visual output relating to a detection, diagnosis, or prognosis. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1605. The algorithm can, for example, determine a presence or absence of a cancer or cancer parameter based on a set of input sequencing data from a sample derived from a subject.

### EXAMPLES

### Example 1: Analysis of Cell Free DNA

The PredicineBEACON assay is able to provide highly sensitive baseline profiling using PredicineWES+ and LP-WGS using blood or urine samples.

Somatic mutations are identified by whole exome sequencing across 20,000 genes accompanied by boosted sequencing of 600 cancer-related genes in the treatment-naive tissue, blood, or urine samples. Based on the sequencing between 16-50 personalized somatic mutations or fusions are selected for each patient. This personalized panel together with a fixed MRD core panel is utilized for MRD detection and monitoring treatment efficacy. The fixed MRD core panel enables detecting novel and actionable mutations beyond variants identified in the baseline, which is critical for treatment monitoring, studying drug resistance, and guiding personalized therapies. Based upon this multi-layer panel design, the mutant allele fraction (MAF) limit of detection (LOD) for PredicineBEACON can reach 0.005%. At each MRD monitoring timepoint, 2 tubes of blood (20 ml total) or 40 ml urine (30-60 ng cfDNA input amount) is recommended, and an ultra-deep sequencing (e.g. 100,000×) can be used for high detection sensitivity. A companion low-pass whole genome sequencing (LP-WGS) is performed to monitor CNV changes for both baseline and follow up timepoints. To establish a baseline for detection of MRD with cfDNA, the PredicineBEACON process starts with a PredicineWES+ panel, which is an enhanced WES panel targeting 20,000 genes with boosted sequencing of 600 specific cancer-related genes. The PredicineWES+ panel also covers important DNA fusions, and includes genome-wide Single Nucleotide Polymorphism (SNP) skeleton probes for Loss of Heterozygosity (LOH) and CNV detection.

The PredicineWES+ cfDNA assay comprises deep sequencing (20,000×, 0.25% LOD) on 600 cancer-related genes and DNA fusions. The remaining whole exome coverage is at an average 2,500× to enable genomic profiling at 1% LOD.

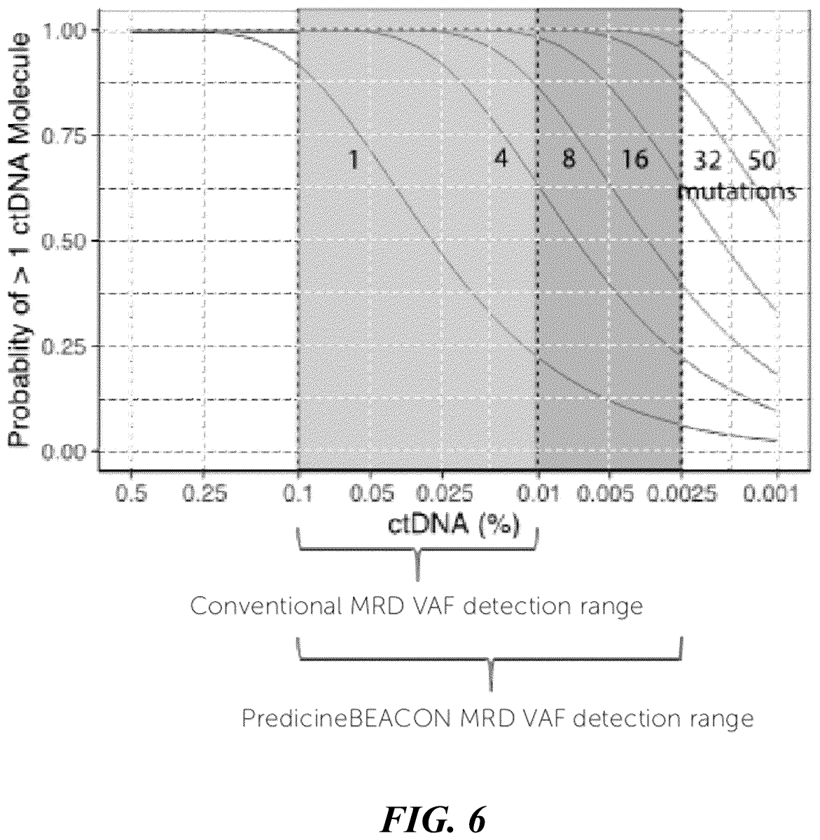

The clinical sensitivity of PredicineBEACON for MRD detection is dependent on the number of tracked somatic mutations. FIG. 6 shows the relationship between tumor fraction and MRD sensitivity based upon different number of patient mutations traced by the assay. The model is based on the possibility of detecting more than 1 single-stranded ctDNA molecule using 30 ng of cfDNA input. Based upon this statistical model, more than 4 effective mutation targets are required to reliably detect ctDNA at 0.05% mutation allele frequencies (MAF), and 16 or more effective mutation targets are needed for detecting ctDNA at 0.01% MAF. Based upon these data, a conventional MRD assay that targets 16, or fewer, mutations will have a detection range of 0.1 to 0.01. With PredicineBEACON targeting between 16 to 50 mutations, the MRD detection range can be improved to 0.0025%.

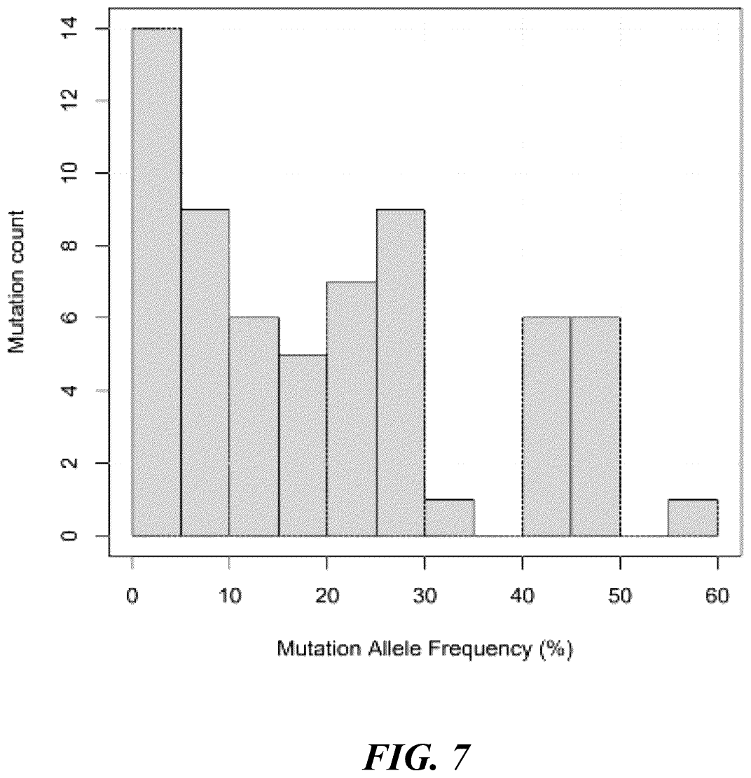

In real clinical samples, the tumor MAFs are often distributed in a wide spectrum. FIG. 7 shows a typical MAF distribution from one clinical sample. This means some high MAF mutations are easier to be detected in MRD tracing, while many others with low MAFs are more difficult to be detected. Considering this factor, the MRD assay needs to target many more mutations than the effective mutation numbers shown in FIG. 6.

Although using more targeted mutations can result in higher MRD detection sensitivity, the specificity of an assay must also be considered. Assay specificity is a significant consideration for baseline-agnostic assays because it needs to detect tens of thousands of unknown mutations covered by the panel. For a baseline-informed MRD assay, such as PredicineBEACON, the risk of a false positive is smaller a concern because the detection focuses on known mutations detected in the baseline sample. However, due to multiple testing issues, the false positive rate still needs to be considered with an increased number of targeted mutations. To control the false positive rate of MRD detection, the PredicineBEACON assay traces up to 50 known baseline mutations, and at least two known mutations are required to be detected to call an MRD event. Based upon titration experiment results using SeraCare reference material, the PredicineBEACON assay is able to achieve a high degree of sensitivity with an acceptable false positive rate (<1%) when tracing 50 baseline mutations.

Personalized probes enable highly sensitive MRD detection. However, should novel mutations become induced during treatment, the personalized probes may fail to detect these. To meet such needs, PredicineBEACON can include a fixed core panel. The fixed 100 kb core panel can covering over 500 hotspots or actionable mutations in about 200 genes. Copy number change is another important cancer driver mechanism. Therefore, PredicineBEACON can include a companion low-pass whole genome sequencing (LP-WGS) performed for both baseline and follow-up longitudinal samples. LP-WGS can further enhance MRD detection sensitivity, especially for patients with few point mutations while having genome-wide copy number changes.

MRD detection often requires detecting trace amounts of ctDNA (<0.1%) through ultra-deep sequencing. As the MAF proportions of ctDNA in MRD monitoring samples are often below the sequencing and PCR error rate, differentiating true mutations from sequencing and PCR errors is required. Using Unique Molecular Identifiers (UMI) can enable identifying reads from the same single strand molecules and suppressing sequencing and late-stage PCR errors during the consensus fragment building process. However, single-stranded UMIs may be unable to differentiate early stage PCR errors from real mutations. A dual UMI design can help recognize single-stranded molecules from the same double-stranded origin, and can thus suppress the early-stage PCR errors. Importantly, a dual UMI design may be infeasible for an amplicon-based NGS assay, which is utilized by some other MRD assays.

By using a dual UMI adapter design, both sequencing and PCR errors (including early-stage PCR errors) can suppressed during the double-strand consensus molecule building step. The PredicineBEACON assay can use one double-stranded ctDNA molecule to make a confident MRD variant call. In the absence of a dual UMI adapter, two or more supporting mutant fragments may be used to an MRD variant call.

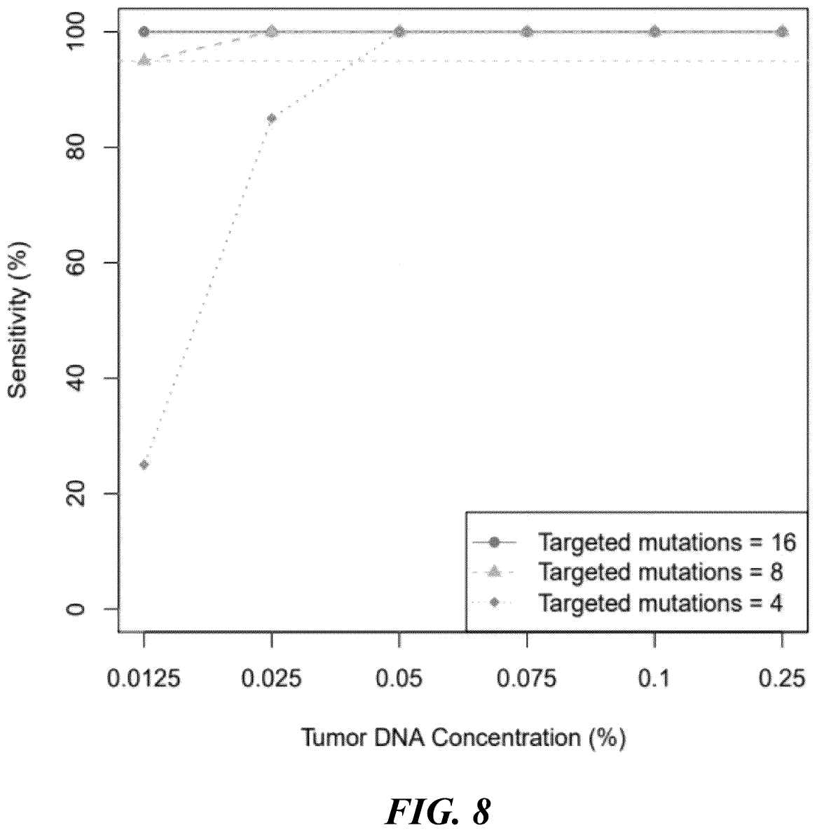

Analytical validation of PredicineBEACON was perform and based on two sets of titration samples: commercial reference material and real-world clinical patient blood samples. 0.025% to 0.0125% MAF, with up to 20 replicates at each titration level. Because the mutations in SeraCare reference material have the same expected MAFs, it is ideal for evaluating the relationship between number of targeted mutations and MRD detection sensitivity. Among those mutations covered by the MRD core panel, 16 mutations were selected for MRD performance evaluation. FIG. 8 shows the MRD detection sensitivity at different titration levels based on DNA input.

TABLE 2
Summary of MRD detection performance by tracing
16 mutations based on SeraCare reference material

| Tumor DNA | | Sensitivity Per | CV of Median | Average |
|---|---|---|---|---|
| Concentration | Replicate | Sample | MAF | Detected |
| (%) | Number | (%) | (%) | Variants |
| 0.25 | 4 | 100 | 2.43 | 16 |
| 0.1 | 8 | 100 | 14.93 | 15.6 |
| 0.075 | 8 | 100 | 16.51 | 15.1 |
| 0.05 | 20 | 100 | 15.79 | 13.2 |
| 0.025 | 20 | 100 | 27.46 | 9.0 |
| 0.0125 | 20 | 100 | 17.86 | 5.9 |

To further evaluate the PredicineBEACON performance near real-world settings, analytical validation was performed using three titration sets of blood samples from patients with cancer. Three metastatic castrate resistant prostate cancer (mCRPC) blood samples were sequenced by PredicineWES⁺ as baselines. Matched PBMC control samples were sequenced to remove CHIP, germline and background variants. Fifty somatic mutations were selected for personalized probes for each patient sample. To mimic real MRD monitoring patient samples with low MAFs, cfDNA from the patient samples were spiked into 30 ng cfDNA from healthy donors. The titration ratio was calculated based on estimated tumor fraction (Maximum MAF after excluding variants with CNV changes) of the blood samples. The patient blood sample was titrated from 0.025%, 0.01%, 0.005% to 0.0025% MAFs with 4 replicates at each titration levels. A pooled panel of 50 personalized probes and MRD core panel was used for MRD profiling. Table 3 shows the summary of MRD detection results.

TABLE 3
Summary of MRD detection performance based
on titration of patient blood samples

| Tumor DNA | | Median | Sensitivity | Average |
|---|---|---|---|---|
| Concentration | MAF Range | MAF | per Sample | Detected |
| (%) | (%) | (%) | (%) | Mutations |
| 0.05 | 0.0038-0.05 | 0.026 | 100 | 17.3 |
| 0.025 | 0.0019-0.025 | 0.013 | 100 | 10.8 |
| 0.01 | 0.0008-0.01 | 0.0052 | 100 | 4.88 |
| 0.005 | 0.0004-0.005 | 0.0026 | 100 | 3.35 |
| 0.0025 | 0.0002-0.0025 | 0.0013 | 50 | 1.5 |

By using PredicineBEACON assay, MRD events can be detected in samples with a tumor fraction as low as 0.005%. The real MRD detection sensitivity depends upon the number of traceable somatic mutations and the distribution of MAFs. Reproducibility was calculated as the percent coefficient of variation (% CV) of the median MAF of positive targets (Table 2 and 3)

The PredicineBEACON assay enables establishing MRD baselines utilizing blood or urine samples, without the need for a baseline tissue sample, which greatly extends the clinical applications of MRD testing. A high-level of MRD detection sensitivity is achieved by targeting up to 50 personalized mutations probes for each patient in addition to analysis of a 100 kb core panel and LP-WGS to assess CNV changes. Additionally, a high-level of MRD detection specificity is achieved through the utilization of double-stranded molecules identified by dual UMIs. Based upon the results of titration experiments using real patient plasma samples, MRD detection with PredicineBEACON can reach a MAF limit of detection as low as 0.005%, improving upon the performance of PCR amplicon-based MRD assays.

The fixed MRD core panel and accompanying LP-WGS enables PredicineBEACON to have potential clinical applications including guiding therapy selection for MRD positive patients, predicting the likelihood of relapse at the time of diagnosis, monitoring response to neoadjuvant treatment, detecting minimal residual disease, monitoring for recurrence after adjuvant treatment, monitoring for treatment resistance, and even the opportunity to treat disease when it is at the MRD stage. Additionally, serial ctDNA detection at different time points by PredicineBEACON during a treatment trial may be used as a measurement of treatment response.

PredicineBEACON integrates a tissue-agnostic, ultra-sensitive MRD variant detection along with actionable guidance on potential next treatment options, providing the first truly actionable MRD test for patients with cancer.

### Example 2: Development and Clinical Application of PredicineBEACON™ Next-Generation Minimal Residual Disease (MRD) Assay for Genitourinary Cancers

Tumor-informed minimal residual disease (MRD) analysis has previously been evaluated in muscle invasive bladder cancer (MIBC) patients undergoing neoadjuvant immunotherapy (NAT). However, many methods require use of tumor tissue and do not provide actionable insights. In this study, a tumor-agnostic MRD assay (PredicineBEACON™) is developed with high sensitivity and capability to detect actionable mutations and genome-wide copy number variations in blood- or urine-based circulating tumor DNA.

The PredicineBEACON™ tumor-agnostic MRD assay includes three components: 1) tissue- or liquid biopsy-based baseline mutation identification and personalized variant panel design, 2) ultra-deep next-generation sequencing of personalized cancer variants and actionable variants, and 3) assessment of genome-wide copy number variations. In brief, PredicineWES+ whole exon sequencing (WES) with boosted depth in 600 cancer-related genes is performed using baseline tumor tissue or liquid biopsy (blood or urine) to identify somatic mutations. This enables ultra-deep sequencing (e.g., 100,000×) of up to fifty personalized somatic mutations along with a fixed core panel of 500 actionable/hotspot variants. In addition, low-pass whole-genome sequencing (LP-WGS) is performed to monitor the MRD by the copy number burden (CNB) calculated from the genome-wide copy number alterations.

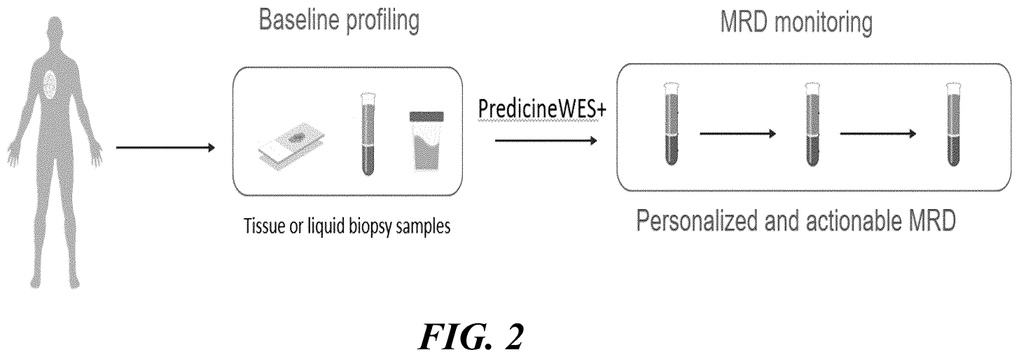

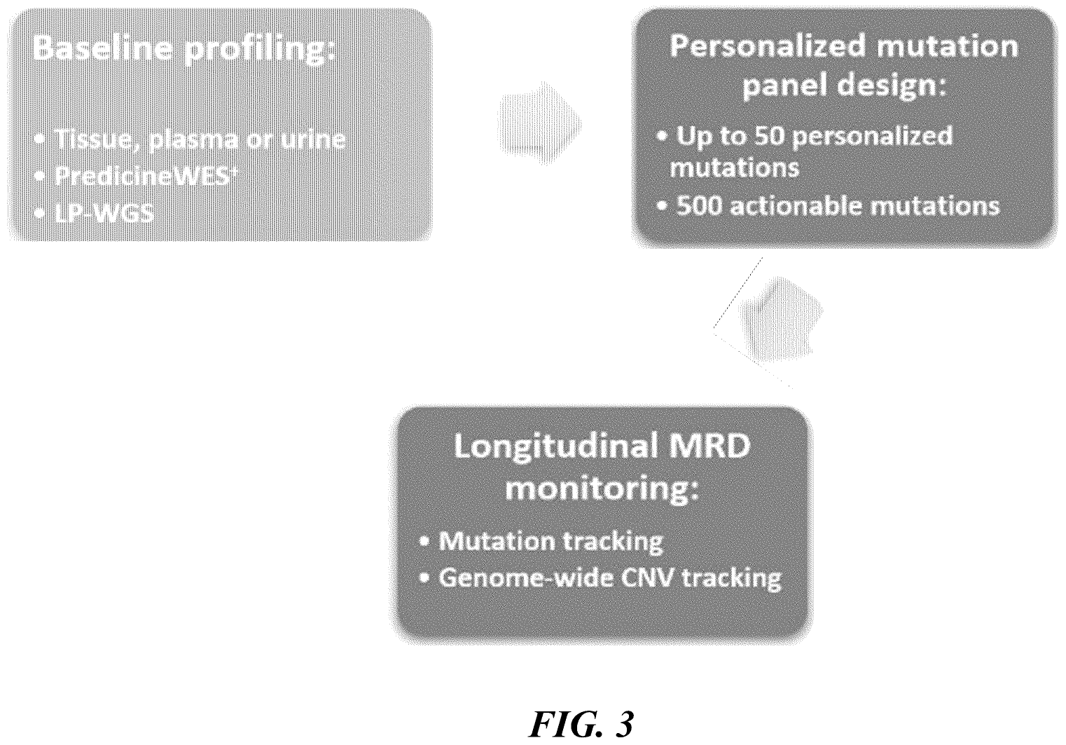

In this study of patients with MIBC, urine samples collected at baseline were analyzed to generate personalized variant profiles. Urine samples were then collected during and tested with the PredicineBEACON™ MRD assay (FIG. 2). FIG. 2 shows a workflow for PredicineBEACON Tissue agnostic, actionable MRD assay.

Plasma cfDNA from cancer patients was diluted in normal cfDNA background at five tumor fraction levels: 0.05%, 0.025%, 0.01%, 0.005%, 0.0025%. 32 somatic mutations were selected from baseline and used for MRD tracking. The assay reached 100% sensitivity with a tumor fraction greater than 0.005%.

TABLE 4
Mutation-detection sensitivity of PredicineBEACON ™ MRD
assay.

| Number of mutations | | Tumor | Tumor | Tumor | Tumor | Tumor |
|---|---|---|---|---|---|---|
| traced for MRD | | fraction | fraction | fraction | fraction | fraction |
| detection | | 0.0025% | 0.005% | 0.01% | 0.025% | 0.05% |
| 32 | Average number | 1.5 | 3.75 | 5.75 | 12.5 | 20 |
| | of mutations | | | | | |
| | detected | | | | | |
| | Sensitivity | 50% | 100% | 100% | 100% | 100% |

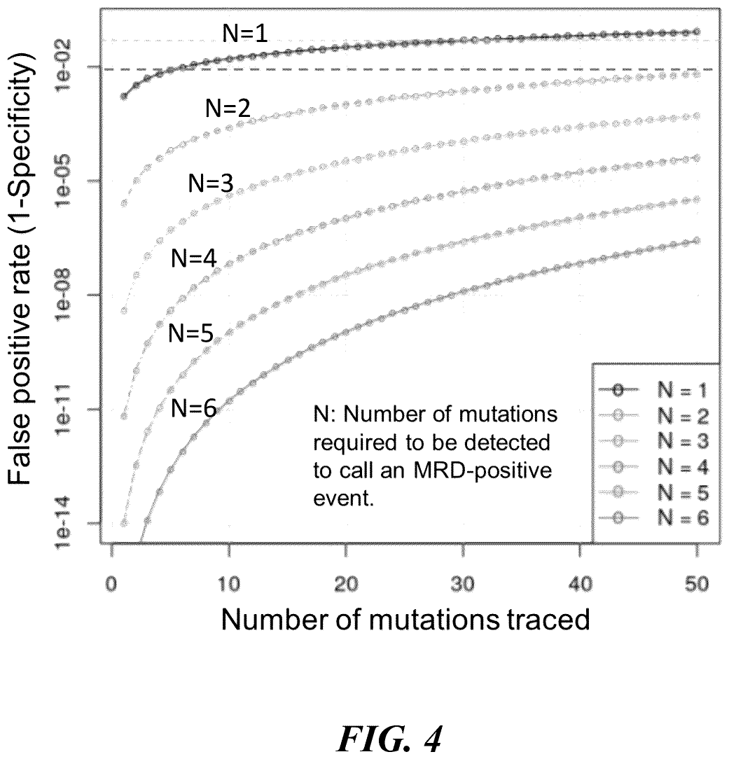

FIG. 4 shows the specificity of mutation detection in PredicineBEACON™ MRD assay.

To evaluate the specificity of mutation calling, plasma samples from healthy donors were tested with the PredicineBEACON™ MRD assay. The assay reached >99% specificity when 2 or mutations were required to be detected to call a mutation MRD-positive event by tracking up to 50 mutations.

Urine-Based PredicineBEACON™ MRD Detection in MIBC Cancer Patients:

Four patients with MIBC undergoing NAT were tested with the PredicineBEACON™ assay. Urine samples were collected before and after neoadjuvant therapy. For MRD testing, urine samples collected before NAT were treated as the baseline and tested with PredicineWES+ to generate a personalized profile of 50 somatic variants that were selected for mutation tracking along with a fixed core of 500 actionable/hotspot variants. LP-WGS sequencing was performed for genome-wide copy number analysis. Tumor fraction inferred from the mutation test and copy number burden (CNB) derived from the LP-WGS test both correlate with the clinical response during NAT.

### Example 3

Muscle-invasive bladder cancer (MIBC) is a challenging disease with dismal prognosis. At present, standard of care for resectable MIBC is neoadjuvant chemotherapy followed by radical cystectomy (RC), which suffers from prevalent contraindications and high rates of tumor recurrence. Because immune checkpoint inhibitors (ICIs) have shown durable efficacy and favorable side-effect profiles compared to cytotoxic drugs², perioperative immunotherapy is emerging as a promising treatment modality, especially in patients who are ineligible or repellant to receive cisplatin. Several ICIs, including pembrolizumab, atezolizumab, durvalumab, and nivolumab, are being evaluated as monotherapy or in combination in the neoadjuvant setting. However, established biomarkers are currently lacking to identify individuals likely to benefit.

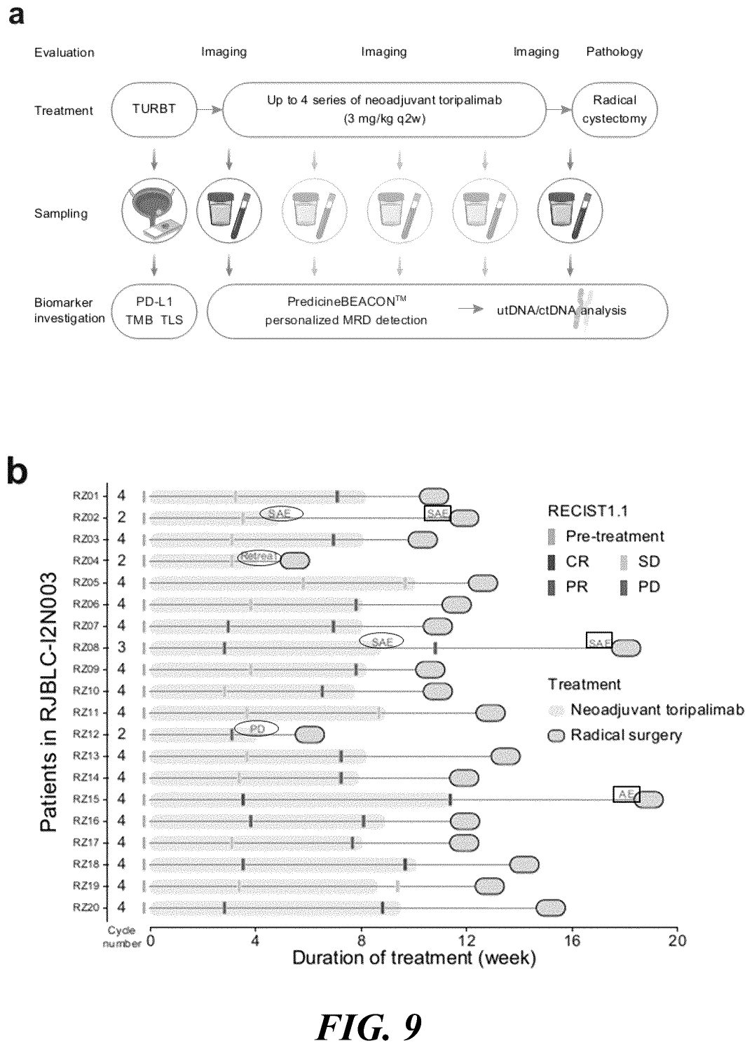

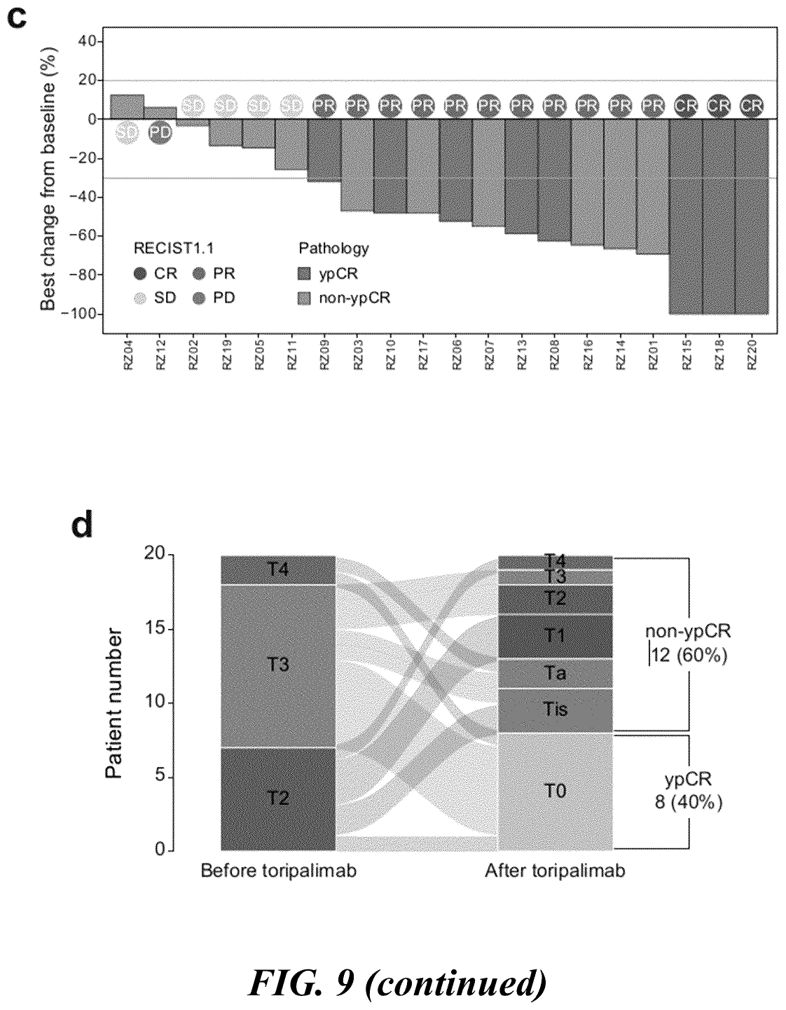

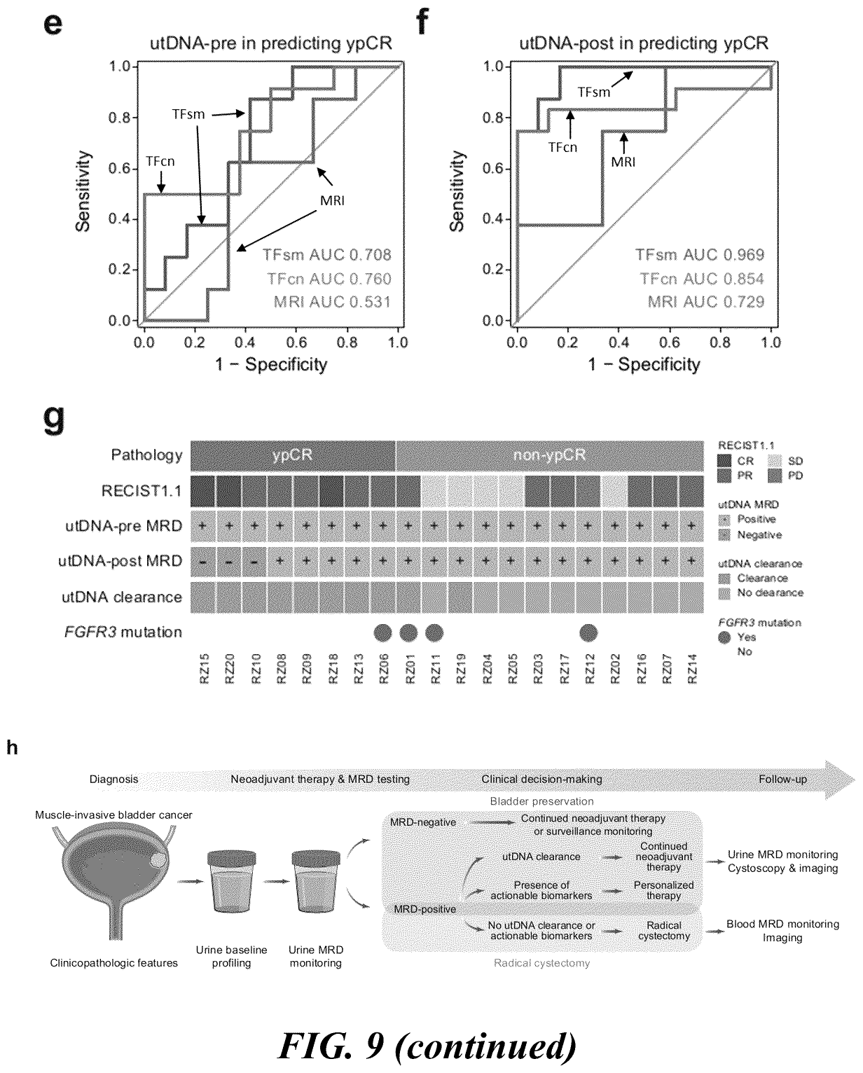

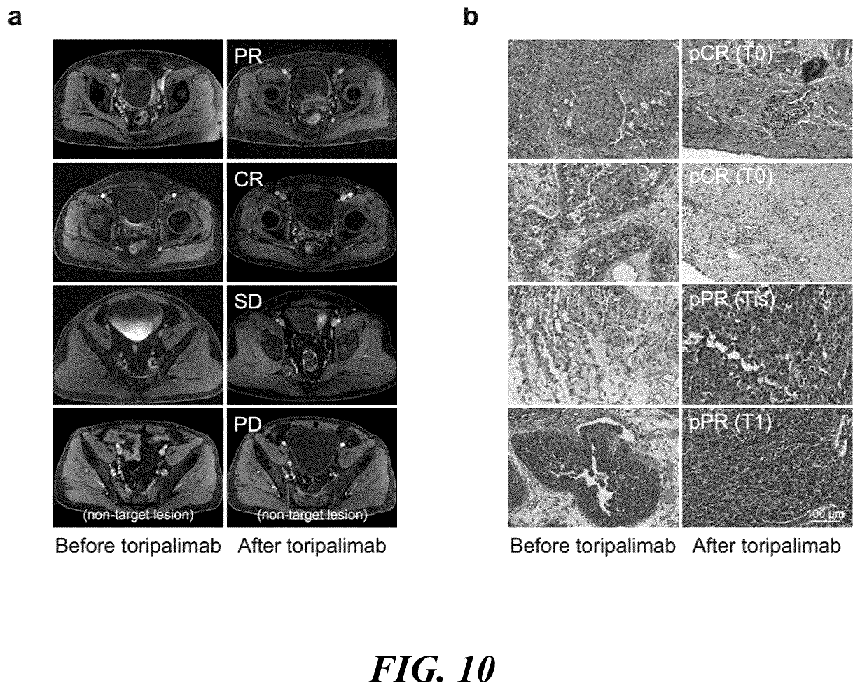

Toripalimab is a humanized IgG4 monoclonal antibody against programmed cell death 1 (PD-1), which has been approved for second-line use in metastatic urothelial carcinoma. To further test the tolerability and effectiveness of toripalimab given to MIBC patients before surgery, a trial was conducted. A total of 20 patients with newly diagnosed T2-4N0M0 MIBC were recruited (Table 5), and treated with up to 4 cycles of toripalimab (3 mg/kg) every 2 weeks prior to RC (FIG. 9A). In general, safety signals were consistent with previous findings (Table 6). Nineteen (95%) and six (30%) patients experienced treatment- and immune-related adverse events (AE), respectively. Grade 3 AEs occurred in three patients (15%), which caused toripalimab discontinuation in two cases. No grade 4 or 5 AEs were observed. All 20 patients were successfully subjected to RC with a median interval of 5.3 weeks (interquartile range, 4.1-6.5 weeks) since last dose. Three patients (15%) had postponed operations due to toxicities (FIG. 9b). Upon toripalimab usage, most patients displayed extensive tumor regression at radiographic (FIG. 9c; FIG. 10.A) and histopathological (FIG. 9D; FIG. 10B) examination with few exceptions. Eight patients (40%) achieved a pathological complete response (ypCR, defined by pT0N0) and an additional eight (40%) were downstaged to non-muscle invasive lesions (pT1 or less). One patient with locoregional progression while receiving toripalimab (RZ12) was able to undergo definitive resection. During a median postoperative follow-up time of 10.2 months (interquartile range, 7.6-13.1 months), only one patient (RZ17) relapsed with a urethral mass and all treated patients were alive. We concluded that the two co-primary endpoints of RJBLC-I2N003 including safety and efficacy were both met, recapitulating results from other ICI trials in the field⁵⁻⁷.

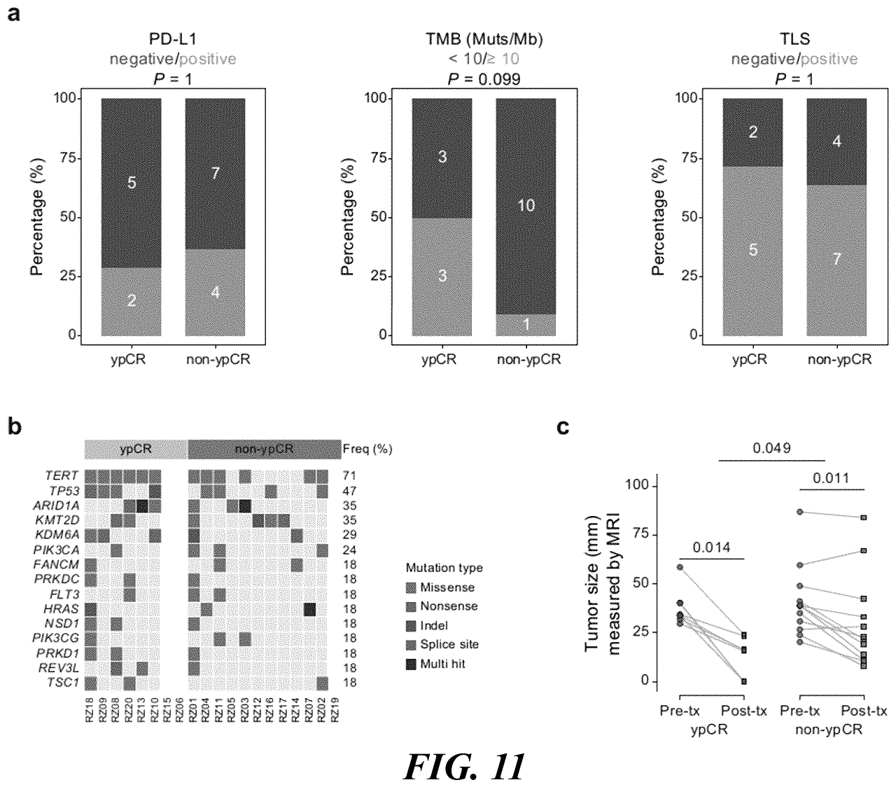

The clinical advantages of preoperative immunotherapy must be weighed against the potential disadvantages of on-therapy tumor progression and adverse events leading to surgical delay. Novel alternatives including targeted agents and antibody-drug conjugates have increasingly become viable options⁸. In addition, bladder preservation is associated with better quality of life, and may be attempted in well-selected cases with complete eradication of malignant cells. Collectively, these considerations emphasize the urgency to develop reliable biomarkers for active disease surveillance and accurate patient stratification throughout the course of neoadjuvant treatment. To this end, prespecified exploratory biomarker discovery was pursued in prospectively collected biospecimens. Measurement of traditional biomarkers at baseline, such as PD-L1 expression, tumor mutation burden, and tertiary lymphoid structures, failed to show predictive value (FIG. 11A). Likewise, whole-exome sequencing (WES) of therapy-naïve neoplastic tissues (Table 7) using PredicineWES+, a WES assay with boosted coverage of 600 cancer-related genes, did not find a statistically significant correlation between specific somatic variants and patient outcome (FIG. 11B). Moreover, radiologic decreases in tumor size measured by repeated magnetic resonance imaging (MRI) only exhibited marginal utility to discriminate pathologic responders (FIG. 19c; FIG. 11C), indicating the pressing need for more robust biomarkers.

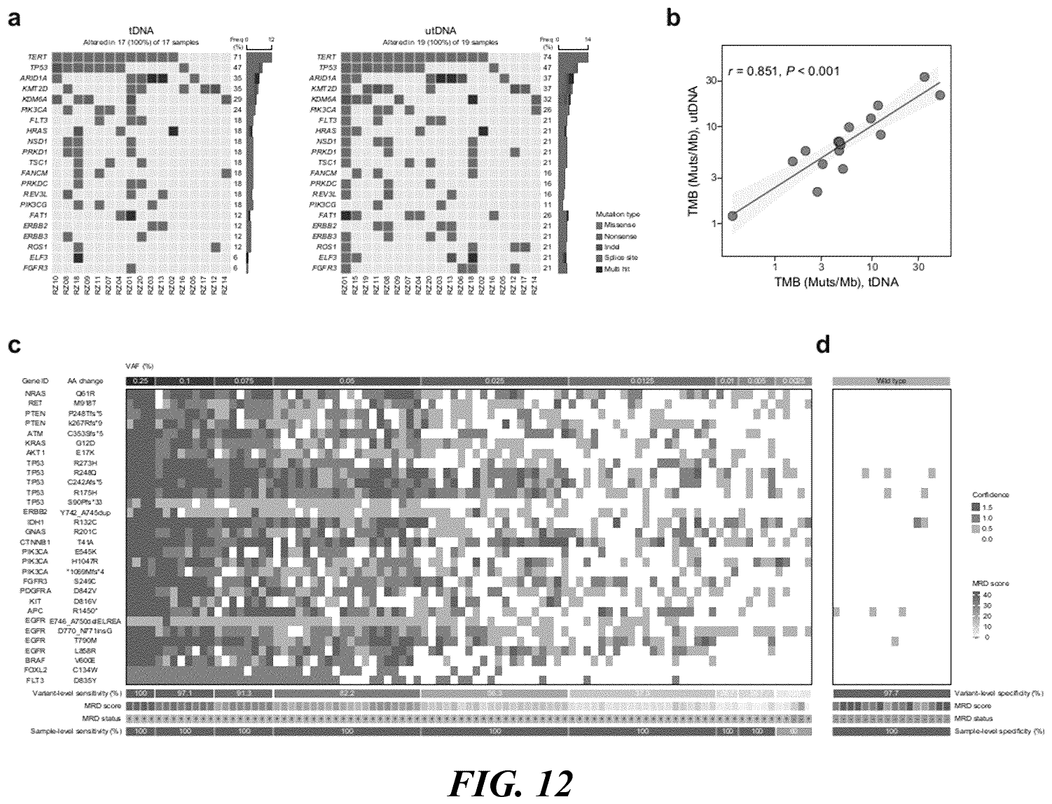

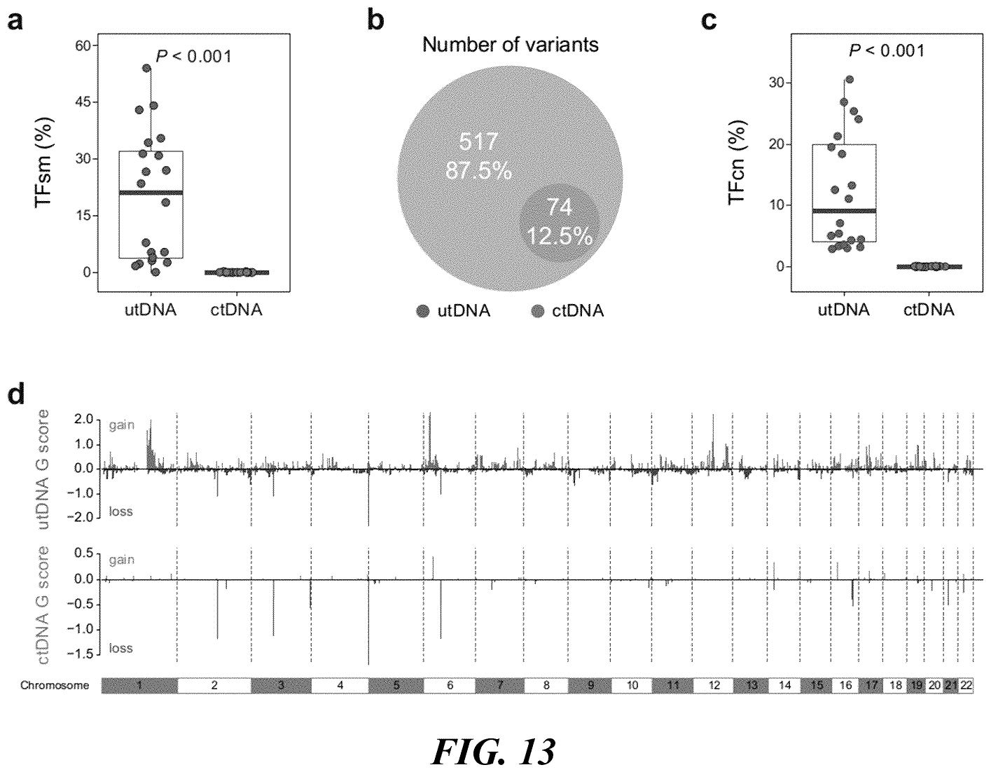

Accumulating evidence suggests that liquid biopsy is instrumental to predict and monitor ICI efficacy⁹. Primary bladder cancer cells shed DNA directly into the urine, and we recently demonstrated that urinary tumor DNA (utDNA) outperformed blood-based circulating tumor DNA (ctDNA) to serve as a dependable and precise surrogate of tumor-derived DNA (tDNA) in MIBC¹⁰. In the present work, we assessed the potential utility of tissue-agnostic urinary minimal residual disease (MRD) analysis to identify true responders to neoadjuvant immunotherapy. Baseline utDNA was subjected to PredicineWES+ for personalized MRD test design (Table 8). Notably, the mutational landscapes derived from utDNA and tDNA profiling were highly similar (FIG. 12A), as were the estimated tumor mutation burdens (FIG. 12B). Given the possibly low abundance of utDNA after transurethral resection of bladder tumor (TURBT) and toripalimab treatment, a PredicineBEACON™ MRD assay was devised that combines ultra-deep sequencing using a bespoke panel to profile up to fifty patient-specific somatic aberrations (Table 9), targeted sequencing of a fixed set of five hundred actionable/hotspot variants (Table 10), and low-pass whole-genome sequencing (LP-WGS) to detect tumor-originated copy number changes. Based on a titration experiment using standard reference materials, PredicineBEACON™ reached 100% sensitivity with a variant allele frequency of greater than 0.005% and >99% specificity to call MRD-positive events (data not shown). We applied PredicineBEACON™ to the RJBLC-I2N003 cohort, and obtained tumor fraction (TF) estimates according to somatic mutations (TFsm) or copy numbers (TFcn) (Table 11. Consistent with our previous observations¹⁰, utDNA showed superior performance than ctDNA to represent urothelial neoplasms, as reflected by evidently higher TFsm (FIG. 13A). In fact, utDNA contained all the substitutions detected in ctDNA (FIG. 13B). In addition, more genome-wide copy number variations were identified in utDNA compared to ctDNA, as assessed by TFcn scores (FIG. 13C) or the GISTIC algorithm (FIG. 13D).

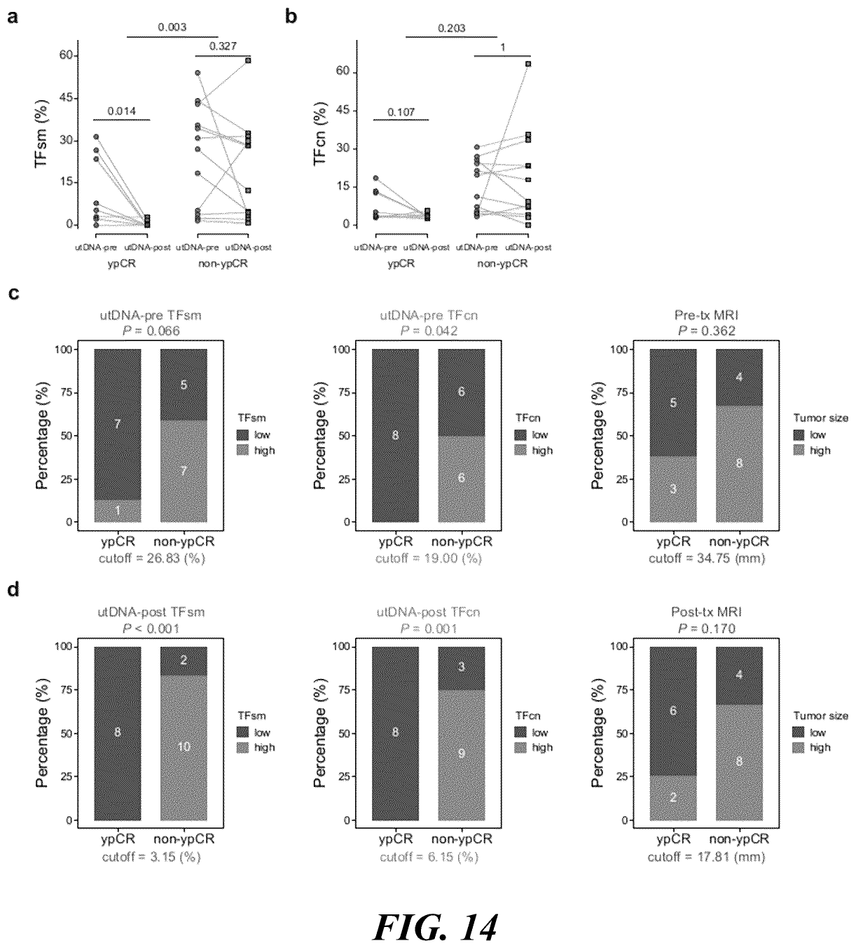

Focusing on quantitative metrics derived from the urine analyte, we found that TFsm was selectively diminished in toripalimab responders (FIG. 14A), as was TFcn, albeit to a modest extent (FIG. 14B), implicating utDNA reduction as a potential biological marker of tumor remission. At baseline, the values of area under the receiver operating characteristic (ROC) curve (AUC) were 0.708, 0.760, and 0.531 for TFsm, TFcn, and MRI measurements, respectively, in predicting ypCR (FIG. 9e). Following completion of neoadjuvant therapy, the AUC values were 0.854, and 0.729, respectively (FIG. 9f). Using the optimal cutoff points defined by ROC analysis, TFsm and TFcn levels in post-treatment utDNA, as compared to those in pre-treatment utDNA (FIG. 14C), were significantly correlated with pathologic outcome (FIG. 14D), and were superior to correlates observed with MRI imaging. Finally, we determined the MRD status of utDNA samples according to the PredicineBEACON™ test. All patients were inferred to be MRD-positive post TURBT, while three (RZ10, RZ15 and RZ20) became MRD-negative before bladder removal and invariably achieved ypCR at the end of the study (FIG. 9g). These findings lay the foundation for tissue-agnostic urinary MRD assessment to identify exceptional responders to neoadjuvant checkpoint blockade who may be candidates for bladder preservation. Such a paradigm-shifting clinical approach will presumably alleviate the significant burden of unnecessary radical cystectomy.

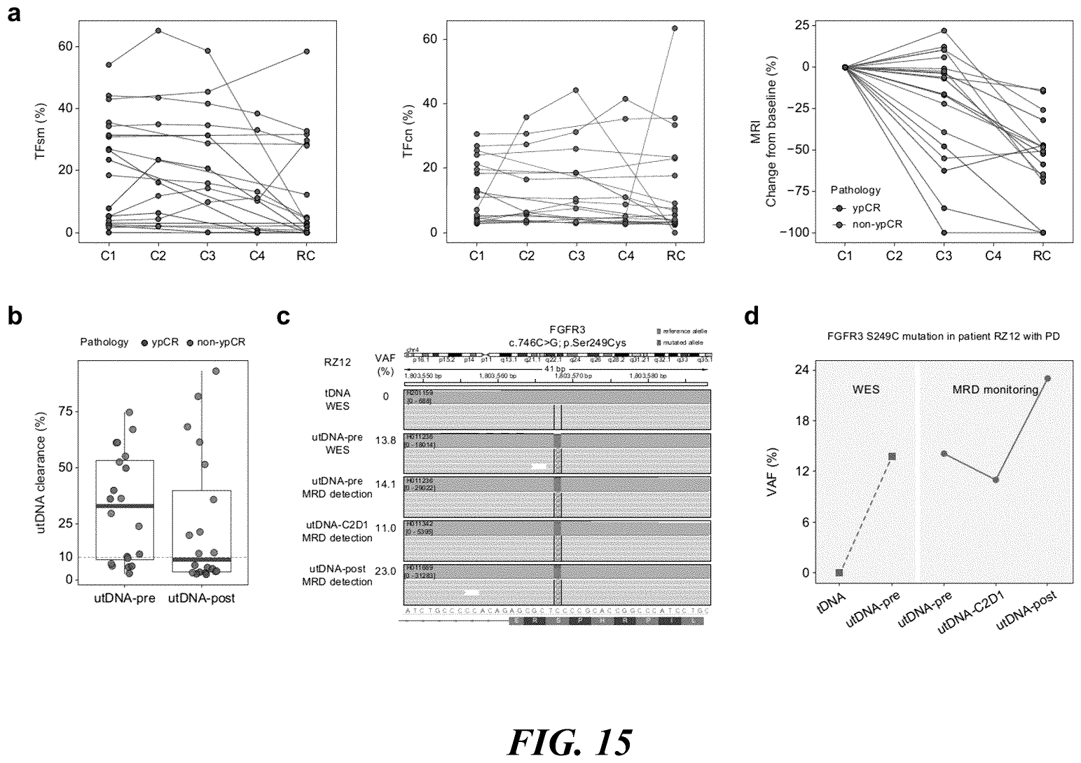

We reasoned that serial utDNA monitoring would aid to gauge the optimal duration of neoadjuvant immunotherapy. Indeed, utDNA kinetic profiles suggested that at least 3-4 cycles of presurgical toripalimab were likely required in most cases to yield dramatic decreases of both TFsm and TFcn (FIG. 15A). Interestingly, utDNA clearance has been observed in 8 out of 8 (100%) of patients that achieved ypCR. Similarly, 7 out of 17 (41%) MRD-positive patients actually showed utDNA clearance with minimal TFsm and TFcn (FIG. 9G; FIG. 15B), and might need continued toripalimab to fully eradicate the remaining cancer cells. Furthermore, to explore the possibility of treating MRD with alternative drugs, we investigated the dynamic changes of actionable variants included in PredicineBEACON™. For example, the recurrent FGFR3 S249C mutation was identified in utDNA samples from patient RZ12 (FIG. 15C), with increased mutant allele frequency observed following neoadjuvant toripalimab (FIG. 15D), in keeping with on-treatment progressive disease. It was worth noting that FGFR3 S249C was not detected in bulky tissue analysis, implying that a subclonal lesion was responsible for tumor growth. These results implicated patient RZ12 as a candidate for subsequent FGFR-targeted therapy, such as erdafitinib. In total, 4 out of 17 MRD-positive subjects (RZ01, RZ06, RZ11, RZ12) could potentially benefit from genotype-matched FDA-approved regimens targeting FGFR3 variants (FIG. 9G; Table 10). Collectively, 14 out of 20 patients in this study (70%, 14/20) could be promising candidates for bladder preservation strategy. Taken together, we propose that longitudinal urinary MRD analysis should be implemented to allow for adaptive management of individual patients (FIG. 9H).

Our data for the first time indicate that neoadjuvant toripalimab followed by RC is feasible and efficacious in patients with localized MIBC. Specifically, neoadjuvant toripalimab had a low incidence of immune-related adverse events, and was not coupled with notable delays or complications in subsequent surgical procedures. The pathological complete response occurred in 40% of enrolled patients, in accordance with the latest clinical investigations of other ICIs in this setting. Therefore, the study supports future exploration of neoadjuvant toripalimab in randomized controlled trials.

The current proof-of-concept biomarker analyses lay unprecedented groundwork for the incorporation of actionable utDNA MRD testing into preoperative MIBC management in upcoming clinical trials and conceivably routine practice. Such a noninvasive approach is poised to not only facilitate individually tailored therapy selection, but also enable real-time surveillance of various treatments including the emerging neoadjuvant immunotherapy, targeted agents and antibody-drug conjugates, among others. We envision that the tissue-agnostic, urine-based MRD assay described here holds enormous promise to inform clinical decision-making around organ-preserving opportunities and may fundamentally transform healthcare guidelines for MIBC patients.

Patients and Study Design

A total of 20 patients with resectable MIBC were analyzed. Patient ages ranged from 18 to 75 years. All patients had an Eastern Cooperative Oncology Group performance status (ECOG PS) of 0-1, and underwent transurethral resection of bladder tumor (TURBT) for tumor resection, pathologic diagnosis, and disease staging. Key exclusion criteria included documented severe autoimmune or chronic infectious disease and use of systemic immunosuppressive medications. Patients were treated with preoperative toripalimab at 3 mg/kg every 2 weeks for up to 4 cycles unless intolerable toxicity or voluntary retreat. Radical cystectomy was planned within 4±2 weeks after the last dose of toripalimab treatment. The primary efficacy endpoint was pathological complete response (ypCR, defined by pT0N0) at surgical resection. Safety was assessed at each patient's clinical visit and documented as per the National Cancer Institute Common Terminology Criteria for Adverse Events, v.4.03. Magnetic resonance imaging (MRI) was performed at baseline and every 2 cycles of toripalimab treatment. Radiographic images were evaluated by clinical investigators according to Response Evaluation Criteria in Solid Tumors v.1.1 (RECIST1.1). The median follow-up time was 10.4 months (interquartile range, 8.0-13.2 months).

Sample Collection and Processing

FFPE (formalin-fixed and paraffin-embedded) tumor samples were obtained and histologically assessed. FFPE with ≥20% tumor content was qualified for DNA extraction and whole-exome sequencing (WES). PD-L1 immunohistochemistry (Dako, CA) was performed and evaluated by certified pathologists and quantified using combined positive score (CPS), i.e., the number of staining-positive cells divided by the total number of viable tumor cells, multiplied by 100. PD-L1 positivity was defined as CPS≥10¹. TLS (tertiary lymphoid structure) was assessed on FFPE tissue sections by hematoxylin and eosin (H&E) staining and TLS positivity was defined as the TLS number ≥1. Before and after neoadjuvant immunotherapy, a volume of 40 ml first-morning urine and a volume of 10 ml peripheral blood were prospectively collected in preservation buffer-prefilled urine collection kits and BD Vacutainer EDTA tubes, respectively. Plasma and buffy coats were separated by centrifugation at 1600×g for 10 minutes followed by 3200×g for 10 minutes at room temperature within 2 hours after collection, and were immediately stored at −80° C.

DNA extraction was performed in a CAP-accredited laboratory (Huidu Shanghai). All collected biospecimens, including urine samples, plasma samples, tumor tissues, and peripheral blood mononuclear cells (PBMCs), were processed for DNA extraction and library preparation. Plasma and urinary cell-free DNA (cfDNA) was extracted using the QIAamp circulating nucleic acid kit (Qiagen). The quantity and quality of purified cfDNA were checked using Qubit fluorimeter and Bioanalyzer 2100. Genomic DNA (gDNA) was extracted from PBMCs and tumor tissues. Up to 250 ng gDNA was enzymatically fragmented and purified.

MRD Assay Design

The PredicineBEACON™ personalized MRD assay included whole exome sequencing of baseline samples using either urine or tumor tissues collected from TURBT, followed by ultra-deep sequencing of subsequent longitudinal urine samples using a personalized MRD panel (personalized mutations plus a fixed panel of actionable/hotspot mutations). Matched PBMC samples were sequenced to obtain high confidence somatic mutation calls. Up to somatic mutations were selected to design a personalized panel for each patient.

Library Preparation and Sequencing

5-30 ng of extracted cfDNA was subjected to library construction including end-repair dA-tailing and adapter ligation. Ligated library fragments with appropriate adapters were amplified via PCR. The amplified DNA libraries were checked using a Bioanalyzer 2100 and samples with sufficient yield were advanced to hybrid capture. Library capture was conducted using Biotin labelled DNA probes. In brief, the library was hybridized overnight with the PredicineBEACON™ panel and paramagnetic beads. Unbound fragments were washed away, and the enriched fragments were amplified via PCR amplification. The purified product was checked on a Bioanalyzer 2100 and then loaded into a Illumina NovaSeq 6000 for sequencing with paired-end 2×150 bp reads.

Analyses of Sequencing Data from gDNA

Sequencing data from gDNA were analyzed using a developed analysis pipeline, which started with the raw sequencing data (BCL files) and culminated in the output of final mutation calls. Briefly, the pipeline first ran adapter trimming, barcode checking and correction. Cleaned paired FASTQ files were aligned to human reference genome build hg19 using the BWA alignment tool. Candidate variants, consisting of point mutations, small insertions and deletions, and structural variations, were identified across targeted regions covered by the PredicineBEACON™ panel.

Analyses of Sequencing Data from cfDNA

NGS Data were analyzed using the Predicine DeepSea NGS analysis pipeline, which started with the raw sequencing data (BCL files) and culminated in the output of final mutation calls. Briefly, the pipeline first performed adapter trim, barcode checking, and correction. Cleaned paired FASTQ files were aligned to human reference genome build hg19 using the BWA alignment tool. Consensus bam files were then derived by merging paired-end reads originated from the same molecules (based on mapping location and unique molecular identifiers) as single strand fragments. Single strand fragments from the same double strand DNA molecules were then further merged as double stranded. By using an error suppression method described previously², both sequencing and PCR errors were mostly corrected during this process.

Candidate variants were called by comparing with local variant background (defined based on plasma and urine samples from healthy donors and historical data). Variants were further filtered by log-odds (LOD) threshold³, base and mapping quality thresholds, repeat regions and other quality metrics.

Candidate somatic mutations were further filtered on the basis of gene annotation to identify those occurring in protein-coding regions. Intronic and silent changes were excluded, while mutations resulting in missense mutations, nonsense mutations, frameshifts, or splice site alterations were retained. Mutations annotated as benign or likely benign were also filtered out based on the ClinVar database₄, or common germline variant databases including 1000 genomes ^{5,6} ExAC⁷, gnomAD and KAVIAR⁸ with population allele frequency >0.5%. Finally, hematopoietic expansion-related variants that have been previously described, including those in DNMT3A, ASXL1, TET2 were also filtered out.

MRD Call

To detect a known variant selected for MRD tracking in the following time points, at least one fragment with confident variant support was required. An MRD variant without double-stranded fragment support was categorized as low confidence. To call a sample as MRD positive, one of the following criteria should be met: (1) three or more low confidence MRD variants were detected, or (2) two or more MRD variants were detected, and one of them had double-stranded variant support.

Tumor Fraction Estimation

The tumor fraction was estimated according to the somatic mutations detected from the MRD assay (TFₛₘ) or the copy numbers detected from the low-pass whole genome sequencing (LP-WGS) assay (TF_{cn}). The TFₛₘ was estimated based on the allele fractions of autosomal somatic mutations:

$${TF}_{sm} = {TF}_{b} \frac{{\sum }_{i}^{n} {m}_{i}}{{\sum }_{i}^{n} {t}_{i}} / \frac{{\sum }_{i}^{n} {m}_{bi}}{{\sum }_{i}^{n} {t}_{bi}}$$

where, TF_{b} is the tumor fraction of the matched baseline sample, i is the selected mutation site for MRD tracking, n is the total number of selected mutation sites for MRD tracking, m is the number of mutated fragments at the mutation site, and t is the total number of fragments at the mutation site; m_{b} and t_{b} are mutated and total fragments at the mutation site at the baseline level, respectively.

The TF_{cn} was estimated using ichorCNA as described previously. Briefly, low-pass whole genome sequencing (LP-WGS) with an overall average coverage of 5× was performed on patient samples. The ichorCNA algorithm was applied to GC and mappability-normalized reads to estimate copy number variations using hidden Markov model (HMM). Then, the TF_{cn} prediction was performed using the ichorCNA R software package.

R version 4.0.0 (https://www.R-project.org) was used for statistical analysis and graphic plotting with ggplot2 and ComplexHeatmap package. The R package pROC was used to perform ROC analysis. The optimal cutoff point was defined based on the greatest Youden's index (sensitivity+specificity−1). The copy number G-score was calculated by the GISTIC 2.0 pipeline via GenePattern (https://www.broadinstitute.org/cancer/software/genepattern). The Wilcoxon rank sum test or Student's t-test was used to compare numeric variables. Fisher's exact test was used to compare categorical variables. All tests were two-sided and considered statistically significant at P<0.05.

### Tables

TABLE 5
Clinicopathological characteristics of 20 patients in our study.

| | | Age at | | | | |
|---|---|---|---|---|---|---|
| | | entry, | height, | weight, | BMI, | |
| Patient ID | Gender | years | cm | kg | kg/m² | ECOG |
| RZ01 | Male | 56 | 165 | 56.5 | 20.8 | 0 |
| RZ02 | Male | 39 | 170 | 69.5 | 24.0 | 0 |
| RZ03 | Male | 70 | 174 | 69.5 | 23.0 | 0 |
| RZ04 | Female | 69 | 163 | 50.5 | 19.0 | 0 |
| RZ05 | Male | 64 | 169 | 59.5 | 20.8 | 0 |
| RZ06 | Male | 63 | 168 | 67 | 23.7 | 0 |
| RZ07 | Male | 59 | 175 | 82 | 26.8 | 0 |
| RZ08 | Male | 64 | 174.5 | 76.5 | 25.1 | 0 |
| RZ09 | Female | 64 | 159 | 52.5 | 20.8 | 0 |
| RZ10 | Male | 63 | 172 | 67 | 22.6 | 1 |
| RZ11 | Male | 65 | 175 | 89 | 29.1 | 0 |
| RZ12 | Male | 63 | 173 | 66 | 22.1 | 0 |
| RZ13 | Male | 61 | 178 | 74 | 23.4 | 0 |
| RZ14 | Male | 62 | 170 | 80.5 | 27.9 | 0 |
| RZ15 | Male | 59 | 165 | 67 | 24.6 | 0 |
| RZ16 | Male | 61 | 181 | 76 | 23.2 | 0 |
| RZ17 | Male | 57 | 174 | 81.5 | 26.9 | 0 |
| RZ18 | Male | 55 | 170 | 75 | 26.0 | 0 |
| RZ19 | Male | 55 | 167 | 92 | 33.0 | 0 |
| RZ20 | Male | 67 | 172 | 70.5 | 23.8 | 0 |

| | | | | TMB of | | TNM |
|---|---|---|---|---|---|---|
| | | | TMB of | basal | | stage |
| Cisplatin | Smoking | | tumor, | urine, | | at |
| eligible | history | PD-L1 | Muts/Mb | Muts/Mb | TLS | entry |
| Yes | Yes | − | 34.5 | 32.8 | − | cT2N0M0 |
| Yes | Yes | − | 0.4 | 1.2 | − | cT4N0M0 |
| Yes | Yes | − | 4.6 | 5.7 | + | cT3N0M0 |
| Yes | No | + | 4.5 | 7.1 | + | cT3N0M0 |
| Yes | No | − | 4.7 | 6.6 | − | cT2N0M0 |
| Yes | Yes | − | NA | 2.3 | + | cT2N0M0 |
| Yes | Yes | + | 1.5 | 4.4 | + | cT2N0M0 |
| Yes | Yes | − | 11.5 | 16.7 | − | cT3N0M0 |
| Yes | No | + | 12.3 | 8.3 | + | cT4aN0M0 |
| Yes | Yes | − | 4.6 | NA | − | cT3N0M0 |
| Yes | Yes | + | 5.8 | 10.0 | + | cT3N0M0 |
| Yes | No | + | 4.6 | 7.1 | + | cT3bN0M0 |
| Yes | Yes | − | 5.0 | 3.7 | + | cT3N0M0 |
| Yes | Yes | − | 2.7 | 2.2 | − | cT2N0M0 |
| Yes | No | NA | NA | 34.3 | NA | cT3N0M0 |
| No | Yes | − | 3.1 | 4.2 | + | cT2N0M0 |
| Yes | Yes | − | 2.1 | 5.7 | + | cT2N0M0 |
| Yes | Yes | + | 50.1 | 21.3 | + | cT3N0M0 |
| Yes | Yes | NA | NA | 7.1 | NA | cT3N0M0 |
| Yes | Yes | − | 9.7 | 12.2 | + | cT3N0M0 |

| | Cycle | | | | pre- | post |
|---|---|---|---|---|---|---|
| | number | Best | | | treatment | treatment |
| | of | change, | | | tumor | tumor |
| Grade | toripalimab | % | c.BOR | c.Response | size, mm | size, mm |
| High | 4 | −69.23 | PR | responder | 35.1 | 10.8 |
| High | 2 | −3.34 | SD | non-responder | 86.9 | 84 |
| High | 4 | −47.20 | PR | responder | 41.1 | 21.7 |
| High | 2 | 12.42 | SD | non-responder | 59.6 | 67 |
| High | 4 | −14.69 | SD | non-responder | 38.8 | 33.1 |
| High | 4 | −52.37 | PR | responder | 33.8 | 16.1 |
| High | 4 | −55.10 | PR | responder | 38.8 | 19.12 |
| High | 3 | −62.56 | PR | responder | 29.65 | 15.65 |
| High | 4 | −31.98 | PR | responder | 34.4 | 23.4 |
| High | 4 | −48.28 | PR | responder | 31.9 | 16.5 |
| High | 4 | −25.79 | SD | non-responder | 30.86 | 22.9 |
| High | 2 | 6.03 | PD | non-responder | 26.69 | 28.3 |
| High | 4 | −58.70 | PR | responder | 58.6 | 24.2 |
| High | 4 | −66.49 | PR | responder | 23.87 | 8 |
| High | 4 | −100.00 | CR | responder | 40.37 | 0 |
| High | 4 | −64.63 | PR | responder | 39.02 | 13.8 |
| High | 4 | −48.28 | PR | responder | 20.3 | 10.5 |
| High | 4 | −100.00 | CR | responder | 33.8 | 0 |
| High | 4 | −13.67 | SD | non-responder | 49 | 42.3 |
| High | 4 | −100.00 | CR | responder | 40.1 | 0 |

| post- | | | | | | |
|---|---|---|---|---|---|---|
| treatment | Pathology | | | Interval, | | RFS, |
| TNM stage | evaluation | Pathology | p.Response | weeks | Recurrence | months |
| pTisN0M0 | pTis | pPR | non-ypCR | 4.1 | No | 16.1 |
| pTaN0M0 | downstaging | pPR | non-ypCR | 8.6 | No | 16.7 |
| pTisN0M0 | pTis | pPR | non-ypCR | 3.9 | No | 16.1 |
| pT2aN0M0 | downstaging | pPR | non-ypCR | 3.0 | No | 13.2 |
| pTisN0M0 | pTis | pPR | non-ypCR | 4.1 | No | 13.2 |
| pT0N0M0 | pT0 | pCR | ypCR | 5.1 | No | 13.0 |
| pT4aN0M0 | upstaging | pPD | non-ypCR | 4.4 | No | 10.3 |
| pT0N0M0 | pT0 | pCR | ypCR | 10.9 | No | 8.9 |
| pT0N0M0 | pT0 | pCR | ypCR | 4.0 | No | 7.1 |
| pT0N0M0 | pT0 | pCR | ypCR | 4.7 | No | 13.1 |
| pTaN0M0 | downstaging | pPR | non-ypCR | 5.6 | No | 10.4 |
| pT3bN2M0 | upstaging | pPD | non-ypCR | 3.6 | No | 8.1 |
| pT0N0M0 | pT0 | pCR | ypCR | 6.9 | No | 10.1 |
| pT1N0M0 | downstaging | pPR | non-ypCR | 5.6 | No | 7.7 |
| pT0N0M0 | pT0 | pCR | ypCR | 9.1 | No | 5.3 |
| pT1N0M0 | downstaging | pPR | non-ypCR | 4.9 | No | 5.9 |
| pT1N0M0 | downstaging | pPR | non-ypCR | 5.4 | Yes | 11.7 |
| pT0N0M0 | pT0 | pCR | ypCR | 5.7 | No | 7.4 |
| pT2aN0M0 | downstaging | pPR | non-ypCR | 6.4 | No | 8.9 |
| pT0N0M0 | pT0 | pCR | ypCR | 7.3 | No | 6.5 |

BMI = body mass index; ECOG = eastern cooperative oncology group; PD-L1 = programmed death ligand 1; TMB = tumor mutation burden; TLS = tertiary lymphoid structure; BOR = best overall response; CR = complete response; PR = partial response; SD = stable disease; PD = progressive disease; ypCR = yield-pathological complete response; RFS = relapse-free survival.

TABLE 6
Adverse events associated with toripalimab.

| | n (%) | |
|---|---|---|
| Treatment related adverse events | | |
| Any grade | 19 | (95) |
| Grade ≥3 | 3 | (15) |
| Serious adverse events | 2 | (10) |
| Treatment suspension due to AEs | 4 | (20) |
| Treatment termination due to AEs | 2 | (10) |
| Death due to AEs | 0 | (0) |
| Immune-related adverse events | | |
| Any grade | 6 | (30) |
| Grade ≥3 | 1 | (5) |
| Surgery-related adverse events | | |
| Any grade | 16 | (80) |
| Grade ≥3 | 2 | (10) |

| | Any grade | Grade ≥3 |
|---|---|---|
| | n (%) | n (%) |
| Treatment-related adverse events | | |
| (n = 20) | | |
| Liver dysfunction | 5 (25) | 1 (5) |
| Hyperglycemia | 4 (20) | 1 (5) |
| Urinary tract infection | 3 (15) | 2 (10) |
| Hyperuricemia | 3 (15) | — |
| Hypokalemia | 3 (15) | — |
| Hypothyroidism | 3 (15) | — |
| Upper respiratory tract infection | 3 (15) | — |
| Anemia | 2 (10) | — |
| Basophilia | 2 (10) | — |
| Chest pain | 2 (10) | — |
| Chest tightness | 2 (10) | — |
| Diarrhea | 2 (10) | — |
| Leucopenia | 2 (10) | — |
| Rash | 2 (10) | — |
| Subclinical hyperthyroidism | 2 (10) | — |
| Weight gain | 2 (10) | — |
| Diabetic ketoacidosis | 1 (5) | 1 (5) |
| Amylase increased | 1 (5) | — |
| Blood bilirubin increased | 1 (5) | — |
| Chills | 1 (5) | — |
| Eosinophilia | 1 (5) | — |
| Fatigue | 1 (5) | — |
| Fever | 1 (5) | — |
| Hematuria | 1 (5) | — |
| Hyperkalemia | 1 (5) | — |
| Hyperthyroidism | 1 (5) | — |
| Indigestion | 1 (5) | — |
| Infusion extravasation | 1 (5) | — |
| Neutropenia | 1 (5) | — |
| Renal insufficiency | 1 (5) | — |
| Tachycardia | 1 (5) | — |
| Urinary leukocyte increased | 1 (5) | — |
| Urinary pain | 1 (5) | — |
| Urinary retention | 1 (5) | — |
| Weight loss | 1 (5) | — |
| Immune-related adverse events | | |
| (n = 20) | | |
| Liver dysfunction | 5 (25) | 1 (5) |
| Hypothyroidism | 3 (15) | — |
| Rash | 2 (10) | — |
| Subclinical hyperthyroidism | 2 (10) | — |
| Hyperthyroidism | 1 (5) | — |
| Surgery-related adverse events | | |
| (n = 20) | | |
| Anemia | 15 (75) | — |
| Constipation | 5 (25) | — |
| Liver dysfunction | 5 (25) | — |
| Infection | 2 (10) | — |
| Pain | 2 (10) | — |
| Intestinal obstruction | 1 (5) | 1 (5) |
| Ileal-neobladder fistula | 1 (5) | 1 (5) |
| Limb edema | 1 (5) | — |
| Fatigue | 1 (5) | — |
| Fever | 1 (5) | — |
| Hypoproteinemia | 1 (5) | — |
| Inappetence | 1 (5) | — |
| Intraoperative hypotension | 1 (5) | — |
| Leukocyte count increased | 1 (5) | — |
| Platelet count decreased | 1 (5) | — |

AE = adverse event.

TABLE 7
List of genes with SNVs and InDels of therapy-naive neoplastic
tissues detected by whole exome sequencing.

A1BG, A4GNT, AADACL3, AADAT, AAGAB, AARS, AATK, ABCA1, ABCA13, ABCA4,
ABCA6, ABCA8, ABCB11, ABCB5, ABCC10, ABCC2, ABCC4, ABCC5, ABCC8, ABCC9,
ABCF2, ABHD11, ABHD12B, ABL2, ABLIM2, ABR, ABRA, ACAA1, ACACA, ACACB,
ACAD10, ACADVL, ACAP3, ACCSL, ACE, ACER2, ACIN1, ACOD1, ACSF3, ACSL4,
ACSL6, ACTC1, ACTR3B, ACTR3C, ACTR8, ACVR2A, ADAD2, ADAM10, ADAM2,
ADAM22, ADAM29, ADAM33, ADAMDEC1, ADAMTS1, ADAMTS13, ADAMTS16,
ADAMTS18, ADAMTS19, ADAMTS2, ADAMTS20, ADAMTS7, ADAMTS9, ADAMTSL1,
ADAP2, ADARB2, ADAT1, ADCY10, ADCY2, ADCY3, ADCY5, ADD1, ADGB, ADGRB1,
ADGRE2, ADGRF3, ADGRG4, ADGRG6, ADGRG7, ADGRL3, ADGRV1, AFAP1, AFAP1L2,
AFF1, AFF4, AFG3L2, AGA, AGAP3, AGBL1, AGBL3, AGBL5, AGL, AGO4, AGPAT5,
AHCTF1, AHCYL1, AHNAK, AHR, AIM1L, AIRE, AK5, AKAP1, AKAP10, AKAP12,
AKAP13, AKAP3, AKAP5, AKAP6, AKIRIN2, AKNA, AKR1B1, AKR1C2, AKTIP,
ALDH1A3, ALDH3A2, ALDH5A1, ALDOA, ALDOB, ALG10B, ALK, ALKBH1, ALMS1,
ALOX12, ALOX5AP, ALPP, ALPPL2, ALX1, ALYREF, AMDHD2, AMERI, AMFR,
AMOTL1, AMPD1, AMT, AMY2B, ANAPC1, ANAPC7, ANGPTL7, ANK2, ANK3, ANKAR,
ANKFN1, ANKMY1, ANKRD12, ANKRD17, ANKRD30B, ANKRD34B, ANKRD36C,
ANKRD44, ANKRD50, ANKRD61, ANKRD62, ANKZF1, ANLN, ANO3, ANOS1, ANPEP,
ANXA2R, ANXA9, AOC3, AP1M1, AP2B1, AP2M1, AP3B2, AP4B1, AP4E1, AP5M1, APBA1,
APBB2, APC, APEH, APEX1, APH1A, APOB, APOPT1, APPBP2, AQP11, ARFGAP3,
ARFGEF2, ARFIP1, ARG1, ARHGAP10, ARHGAP20, ARHGAP21, ARHGAP23,
ARHGAP27, ARHGAP32, ARHGAP5, ARHGAP6, ARHGEF1, ARHGEF10, ARHGEF17,
ARHGEF18, ARID1A, ARID2, ARID3C, ARID4A, ARID5B, ARL13B, ARL14, ARMC1,
ARMC4, ARMC9, ARNT, ARPC4, ARPP21, ARSJ, ASB10, ASB13, ASCC2, ASCC3, ASCL3,
ASH1L, ASIC2, ASIC4, ASMTL, ASPM, ASTE1, ASXL1, ASXL2, ATAD2B, ATAD3C,
ATAD5, ATCAY, ATF2, ATG16L1, ATG16L2, ATG2B, ATG9B, ATM, ATP10A, ATP10B,
ATP10D, ATP11B, ATP11C, ATP13A5, ATP1A3, ATP1A4, ATP2A2, ATP2B1, ATP2C1,
ATP6V0D2, ATP6V1C2, ATP8B4, ATP9B, ATR, ATRN, ATXN1L, ATXN2L, ATXN7L1,
ATXN7L3B, AUNIP, AUP1, AUTS2, AXDND1, B3GALNT2, B3GLCT, B4GALT2, BACH1,
BACH2, BAD, BAG6, BAK1, BAMBI, BAP1, BAZ1A, BAZ2B, BBS12, BBX, BCAR1,
BCAR3, BCKDHA, BCL2L12, BCL2L15, BCL6B, BCL7C, BCL9L, BCLAF1, BCORL1, BCR,
BDP1, BHLHE23, BICD1, BIK, BIN2, BIRC3, BIRC6, BLNK, BMP1, BMP3, BMPR2, BNC1,
BNIP3L, BPIFA2, BPTF, BRAF, BRD1, BRD2, BRD8, BRINP1, BRINP2, BRPF1, BRWD3,
BSCL2, BSN, BTBD1, BTBD16, BTBD19, BTBD3, BTF3L4, BTG2, BTN3A3, BTNL2, BUB3,
BUD13, C10orf88, C11orf45, C11orf65, C11orf80, C11orf86, C12orf29, C12orf71, C12orf74,
C14orf37, C15orf39, C15orf41, C15orf52, C16orf70, C17orf50, C17orf75, C19orf68, C19orf71,
C1orf101, C1orf106, C1orf185, C1orf234, C1orf43, C1orf56, C20orf196, C22orf39, C2CD2L,
C2CD4D, C2orf16, C2orf80, C2orf81, C3orf58, C3orf80, C4A, C4orf3, C4orf51, C5, C5orf42,
C6, C6orf141, C6orf222, C7, C7orf25, C8orf22, C8orf33, C8orf34, C9orf131, C9orf50, C9orf57,
CA14, CA8, CA9, CAAP1, CABS1, CACNA1G, CACNA1I, CACNA2D1, CACNA2D2,
CACNA2D4, CAD, CADM1, CAGE1, CALCB, CALCOCO1, CALCR, CALR3, CALY,
CAMKK2, CAMSAP2, CAMSAP3, CAPN13, CAPN14, CAPN3, CAPRIN2, CARD11, CARF,
CARNS1, CASKIN1, CASP10, CATSPERB, CC2D1B, CCAR1, CCDC106, CCDC110,
CCDC112, CCDC117, CCDC127, CCDC129, CCDC136, CCDC141, CCDC148, CCDC150,
CCDC151, CCDC158, CCDC168, CCDC177, CCDC178, CCDC181, CCDC189, CCDC191,
CCDC34, CCDC39, CCDC51, CCDC57, CCDC60, CCDC66, CCDC68, CCDC7, CCDC73,
CCDC80, CCDC88A, CCDC88B, CCDC92B, CCDC94, CCDC97, CCL3L3, CCNB2, CCNB3,
CCNL2, CCPG1, CCT5, CD101, CD109, CD2BP2, CD300LG, CD46, CD47, CD83, CDAN1,
CDC123, CDC14C, CDC25B, CDC37, CDC42, CDC42BPA, CDC42EP1, CDC5L, CDC6,
CDCP1, CDH13, CDH18, CDH24, CDH26, CDH8, CDH9, CDHR1, CDIP1, CDK11A, CDK14,
CDK5RAP3, CDK6, CDKN1A, CDS1, CDYL, CEACAM18, CEBPA, CEBPD, CEBPZ, CECR2,
CELF2, CELSR2, CELSR3, CENPF, CENPK, CENPO, CENPT, CEP126, CEP128, CEP131,
CEP162, CEP170B, CEP192, CEP250, CEP295, CEP350, CEP44, CEP89, CEP95, CER1,
CFAP221, CFAP43, CFAP46, CFAP54, CFAP65, CFAP99, CFH, CFI, CFTR, CHAF1A, CHD2,
CHD7, CHD8, CHDH, CHEK2, CHL1, CHMP4B, CHMP7, CHN2, CHRD, CHRM1, CHRM3,
CHST1, CHST3, CHST5, CIC, CILP, CILP2, CISD3, CISH, CITED2, CKLF, CLASP2, CLCN1,
CLCN6, CLCN7, CLEC11A, CLEC14A, CLECIA, CLIC3, CLIC5, CLK2, CLNS1A, CLTCL1,
CLUH, CLVS2, CMPK2, CMTM4, CMTM7, CNGB3, CNNM1, CNNM3, CNNM4, CNOT1,
CNOT6, CNST, CNTD1, CNTN1, CNTN6, CNTNAP3, CNTNAP5, CNTRL, COASY,
COL11A2, COL14A1, COL15A1, COL22A1, COL24A1, COL27A1, COL4A2, COL4A4,
COL4A5, COL5A1, COL5A2, COL6A1, COL6A3, COL6A5, COL6A6, COL9A1, COPA,
COPG2, COPS5, COPS6, COX10, COX412, CPD, CPEB4, CPNE4, CPNE6, CPSF1, CPSF6,
CPT1A, CPTP, CPXM1, CPZ, CR1L, CR2, CRABP2, CRACR2A, CRAMP1, CRB1, CRBN,
CREBBP, CRELD1, CRELD2, CRISPLD2, CRLF1, CRLS1, CROT, CRY1, CSF1, CSF1R,
CSH2, CSMD1, CSMD2, CSMD3, CSN3, CSNK1E, CSNK1G1, CSNK1G2, CSPG4, CST1,
CTAGE9, CTC1, CTCFL, CTH, CTNND1, CTNND2, CTNS, CTR9, CTSA, CTSH, CTTNBP2,
CUBN, CUEDC2, CUL1, CUL2, CUL4A, CUL7, CUZD1, CWH43, CXCL8, CXXC4, CYB5R2,
CYC1, CYHR1, CYP1A2, CYP26A1, CYP26B1, CYP27B1, CYP27C1, CYP2A7, CYP2D6,
CYP4V2, CYTH1, DAAM1, DAG1, DAO, DAPK2, DARS2, DBT, DBX2, DCAF11, DCAF15,
DCBLD2, DCDC1, DCHS1, DCLK1, DCP2, DCSTAMP, DCTN1, DCTN2, DDR1, DDX20,
DDX27, DDX42, DDX54, DEAF1, DEFA4, DEFB124, DENND1B, DENND4A, DENND4B,
DENND5A, DEPDC4, DEPDC5, DES, DGAT1, DGKB, DGKG, DHH, DHRS13, DHRS2,
DHRS4, DHTKD1, DHX36, DHX58, DHX8, DIAPH2, DICER1, DIP2B, DIS3, DISC1, DISP1,
DLAT, DLC1, DLEC1, DLG1, DLG3, DLGAP5, DLK1, DMBT1, DMD, DMRTB1, DMXL2,
DNAH1, DNAH10, DNAH11, DNAH12, DNAH14, DNAH17, DNAH2, DNAH3, DNAH5,
DNAH6, DNAH7, DNAH8, DNAI1, DNAJB13, DNAJC1, DNAJC11, DNAJC13, DNAJC14,
DNAJC18, DNAJC21, DNAJC5B, DNAJC6, DNAJC8, DNASE1L1, DNHD1, DNMT3B,
DOC2A, DOCK5, DOCK6, DOCK7, DOCK8, DOK1, DOPEY2, DOT1L, DPAGT1, DPY19L1,
DPY19L2, DPY19L3, DPYSL2, DPYSL3, DPYSL5, DRD2, DRD5, DRG2, DSC1, DSCAML1,
DSCC1, DSG2, DSG4, DTX4, DTYMK, DUOX1, DUOX2, DUOXA2, DUS3L, DUSP16,
DUSP2, DUSP28, DUSP5, DVL3, DXO, DYNC1H1, DYNC2H1, DYNLT3, DYRK1A, DZIP1,
E2F1, E2F2, EBAG9, EBF4, EBNA1BP2, ECHDC2, ECT2L, EDEM3, EEA1, EEF1A1,
EFCAB13, EFCAB8, EFHC1, EFL1, EFNA5, EFTUD2, EGFLAM, EGLN1, EGR2, EGR4,
EHD4, EHMT1, EIF1, EIF2AK4, EIF2B1, EIF2B5, EIF3A, EIF3M, EIF4E3, EIF4G3, ELF2,
ELF3, ELK4, ELOA, ELOVL2, ELP3, EMC3, EML6, ENGASE, ENO1, ENO4, ENPEP, ENPP2,
ENPP7, ENTPD1, EPAS1, EPB41, EPB41L2, EPB41L3, EPC1, EPG5, EPHA1, EPHA7, EPHB1,
EPHB4, EPHB6, EPOP, EPOR, EPS15, EPS8L3, EPX, ERAP2, ERBB2, ERBB3, ERBB4,
ERBIN, ERC1, ERC2, ERCC1, ERCC2, ERCC3, ERI1, ERI3, ERICH3, ERICH6, ERICH6B,
ERLIN2, ERMN, ERP44, ESD, ESRP2, ETV2, ETV4, ETV5, EVI2A, EVL, EVPL, EXD3,
EX05, EXOC4, EXOC6B, EXOG, EXOSC10, EXOSC2, EXOSC8, EZH1, F2RL2, F5, F7,
FAAP24, FABP3, FAM102B, FAM105A, FAM111A, FAM120A, FAM120B, FAM122C,
FAM124B, FAM126B, FAM129A, FAM129B, FAM133B, FAM134A, FAM149A, FAM155A,
FAM161B, FAM166B, FAM170A, FAM174B, FAM180B, FAM186A, FAM186B, FAM189A1,
FAM208A, FAM214A, FAM221A, FAM227A, FAM3D, FAM45A, FAM46A, FAM50A,
FAM53A, FAM53C, FAM63B, FAM65A, FAM65B, FAM71F2, FAM72D, FAM78B, FAM83B,
FAM83G, FAM84B, FAM89A, FANCD2, FANCF, FANCI, FANCM, FARP2, FASLG, FASN,
FASTKD1, FAT1, FAT3, FAT4, FBF1, FBLN1, FBN1, FBRS, FBXL13, FBXL18, FBXL2,
FBXL5, FBXL6, FBXO10, FBXO11, FBXO18, FBXO21, FBXO30, FBXO41, FBXW2, FBXW4,
FBXW7, FBXW8, FCGR2A, FCGRT, FCRL1, FCRL3, FDFT1, FEM1A, FEM1C, FER1L6,
FFAR3, FGF12, FGF23, FGF5, FGFR1OP, FGFR3, FGFR4, FHOD3, FIG4, FIGN, FIGNL1,
FIP1L1, FITM2, FKBP4, FLG, FLG2, FLNA, FLNC, FLOT2, FLT1, FLT3, FLT3LG, FMN1,
FMO1, FMO5, FMR1NB, FN1, FNBP1L, FNBP4, FNDC1, FNDC3A, FNDC3B, FNIP1, FNIP2,
FOLH1, FOXA1, FOXE1, FOXH1, FOXK2, FOXN2, FOXO1, FOXQ1, FREM1, FREM2,
FREM3, FRMD4A, FRMD4B, FRMPD2, FRS2, FRS3, FRY, FSD1L, FSIP2, FUZ, FXR1,
FXYD3, FYB, FZD4, FZD5, GOS2, GAA, GABRA4, GAL3ST3, GALC, GALK2, GALNT14,
GALNT4, GALNT8, GANAB, GAPVD1, GATA3, GATA4, GATA6, GATAD2A, GATSL3,
GBF1, GBP1, GCC2, GCGR, GCH1, GDA, GDAP2, GDF11, GFAP, GFM2, GFPT1, GGCX,
GGNBP2, GGT1, GGT5, GHR, GIGYF1, GIN1, GIT1, GJB5, GKN1, GLB1L, GLG1, GLI1,
GLIPR1L2, GLMN, GLT1D1, GLTSCR2, GMCL1P1, GMPR, GNAI2, GNAL, GNAS,
GNPDA2, GNPTAB, GNRH1, GOLGA2, GOLGA4, GOLGA6D, GOLGA6L2, GOLGA7B,
GOLGA8K, GOLGA8M, GOLGB1, GON4L, GPIBB, GPA33, GPAT2, GPATCH1, GPATCH3,
GPATCH8, GPHN, GPR107, GPR137, GPR156, GPR158, GPR179, GPR18, GPR39, GPR68,
GPRC6A, GPS2, GPX8, GRAMD4, GREB1L, GRHL1, GRID1, GRID2, GRIK1, GRIK5,
GRIN1, GRIN3B, GRIP2, GRIPAP1, GRK2, GRM4, GRM6, GRN, GSAP, GSDMB, GSG1,
GSTA1, GTF3C1, GTF3C5, GUCY1A3, GUCY1B3, GYPA, GYS1, H2AFX, H3F3A, H6PD,
HACD3, HACL1, HAND1, HAO2, HARS, HAVCR1, HBP1, HCAR2, HCAR3, HCK, HDAC2,
HDAC7, HDAC9, HEATR5B, HEATR6, HECTD2, HECTD3, HECTD4, HELZ, HELZ2,
HEPHL1, HERC1, HERC3, HES3, HEXIM2, HEY2, HFE2, HGF, HGSNAT, HHATL, HIF1AN,
HIGD2B, HIP1, HIPK1, HIPK3, HIRA, HIRIP3, HIST1H1C, HIST1H1D, HIST1H2AI,
HIST1H2AM, HIST1H2BG, HIST1H2BL, HIST1H3F, HIST1H3G, HIST1H4B, HIST2H2BE,
HIST3H3, HK1, HK2, HKDC1, HLA-C, HLA-G, HLF, HMCN1, HMCN2, HMGCLL1, HMMR,
HMOX1, HNF1A, HNRNPD, HNRNPR, HOOK3, HORMAD2, HOXA6, HOXB1, HOXB13,
HOXB8, HOXC9, HOXD13, HOXD4, HP, HPS6, HPSE, HRAS, HRC, HRCT1, HRH4, HS2ST1,
HS3ST4, HS6ST1, HSBP1, HSCB, HSD11B1L, HSD17B11, HSF1, HSP90AA1, HSP90AB1,
HSPA1L, HSPA4, HSPA6, HSPA8, HSPA9, HSPB1, HSPH1, HTATSF1, HTR2C, HTR3C,
HTRA2, HTT, HUWE1, HYAL1, HYDIN, HYOU1, IAH1, ICE1, ID1, IDE, IDH1, IDH2, IDI1,
IFFO2, IFIT1B, IFIT5, IFNL3, IFT172, IFT22, IFT46, IFT88, IGDCC3, IGDCC4, IGF1, IGF1R,
IGF2R, IGSF1, IHH, IKBKAP, IKZF2, IL1R2, IL1RAPL1, IL20RB, IL21R, IL27RA, IL3, IL33,
IL3RA, IL411, ILK, IMP3, INCENP, INO80, INPP4A, INPPL1, INS-IGF2, INTU, INVS,
IPCEF1, IPO11, IPO5, IPO7, IPO8, IPP, IQCF6, IQCG, IRF1, IRF3, IRF9, IRS1, ISL2, ITFG2,
ITGA2B, ITGA4, ITGA6, ITGAV, ITGB1, ITGB1BP1, ITGB4, ITGBL1, ITPK1, ITPRIP,
ITSN1, ITSN2, IVD, IWS1, IZUMO2, JAG1, JAK1, JAK2, JMJD1C, JRK, JUNB, JUP, KANK3,
KAT2A, KAT6B, KATNAL2, KBTBD4, KCNB2, KCNG3, KCNH6, KCNH8, KCNJ5,
KCNK16, KCNK2, KCNK6, KCNK7, KCNK9, KCNN2, KCP, KCTD16, KDELR1, KDM3A,
KDM3B, KDM5A, KDM5B, KDM6A, KDM6B, KDM7A, KDR, KHSRP, KIAA0232,
KIAA0355, KIAA0430, KIAA0556, KIAA0586, KIAA1024, KIAA1109, KIAA1143,
KIAA1191, KIAA1210, KIAA1211L, KIAA1456, KIAA1462, KIAA2026, KIDINS220, KIF15,
KIF20B, KIF21B, KIF22, KIF5C, KIF9, KIFAP3, KIFC2, KIFC3, KIR2DL3, KIRREL3, KIT,
KIZ, KLF13, KLF5, KLHL1, KLHL21, KLHL24, KLHL3, KLHL31, KLHL34, KLHL41,
KLK13, KLK3, KLKB1, KMT2A, KMT2B, KMT2D, KMT2E, KNSTRN, KNTC1, KPNA6,
KPTN, KRIT1, KRT18, KRT19, KRT23, KRT27, KRT3, KRT78, KRTAP20-2, KRTAP4-9,
KRTAP9-3, KYNU, L3MBTL1, LAD1, LAMA3, LAMB1, LAMB2, LAMB4, LAMC3,
LAMTOR3, LARP4, LARS, LARS2, LATS1, LAX1, LCA5, LDLRAD4, LEF1, LEFTY1,
LEMD3, LENG1, LEPR, LIFR, LINC00116, LIPT1, LMNA, LMO4, LMO7, LMOD2, LMTK2,
LNPEP, LNPK, LOC100128108, LOC101928841, LOC389602, LOXHD1, LPCAT3, LPIN2,
LPL, LPP, LRBA, LRFN4, LRFN5, LRGUK, LRIF1, LRIG2, LRP1, LRP10, LRP1B, LRP2,
LRP4, LRP6, LRPPRC, LRRC14, LRRC29, LRRC3C, LRRC4C, LRRC7, LRRC9, LRRFIP1,
LRRIQ1, LRRK2, LRTM1, LSS, LTB4R, LTB4R2, LTBP3, LTBP4, LTBR, LTF, LTK, LTV1,
LUC7L, LUC7L2, LY6H, LY86, LY9, LYPD3, LYPLA2, LYSMD2, LYST, MACF1, MAEA,
MAGEA10, MAGEB3, MAGEF1, MAGEL2, MALRD1, MAML1, MAML2, MAML3,
MAN2C1, MANBA, MANBAL, MAOA, MAP3K10, MAP3K11, MAP3K15, MAP3K21,
MAP3K5, MAP3K6, MAP4, MAP4K3, MAP4K4, MAPK1, MAPK15, MAPK4, MAPK8,
MAPK8IP1, MAPK8IP3, MAPRE2, MARCHF7, MARCHF9, MARCKSL1, MARK1, MARK2,
MAST3, MAST4, MAT2A, MB21D1, MB21D2, MBD1, MBD2, MBD6, MBTD1, MBTPS1,
MC5R, MCC, MCCC2, MCF2L, MCM3, MCM6, MCM8, MCTP1, MCTP2, MDGA2, MDN1,
MED12, MED12L, MED13, MED13L, MED16, MED18, MED23, MED25, MED8, MEGF10,
MEGF8, MEOX2, MEP1B, MESDC1, MET, METAP1, METAP2, METTL25, METTL3,
MFAP1, MFSD10, MFSD12, MFSD14B, MFSD6, MGAM2, MGAT2, MGAT5B, MIA2,
MICAL1, MID2, MIEF1, MIER3, MIF, MIGA2, MIIP, MINDY4B, MINK1, MIPOL1, MITF,
MKS1, MKX, MLF1, MLH3, MLNR, MLXIP, MMACHC, MMP13, MMP19, MMP25, MMP28,
MMP7, MMRNI, MMS19, MN1, MNS1, MOAP1, MON1A, MON1B, MPHOSPH10,
MPHOSPH8, MPO, MPPE1, MRC2, MRFAP1L1, MROH2B, MROH7, MROH8, MRPL15,
MRPL38, MRPS18B, MS4A4E, MSANTD2, MSL3, MST1L, MST1R, MSX2, MTA1, MTA2,
MTARC1, MTARC2, MTBP, MTHFD1L, MTMR11, MTMR4, MTOR, MTUS2, MTX2,
MUC15, MUC16, MUC17, MUC19, MUC22, MUC5B, MUM1, MUSTN1, MYBBP1A,
MYBPC1, MYCBP2, MYCL, MYEF2, MYH1, MYH14, MYH7, MYH7B, MYL1, MYO10,
MYO15A, MYO15B, MYO16, MYO18B, MYO19, MYO1G, MYO3A, MYO3B, MYO5C,
MYO6, MYO7A, MYO7B, MYO9A, MYOM2, MYOM3, MYRIP, MYSM1, MYT1L, N4BP2,
NAA15, NAALADL1, NAALADL2, NANS, NAP1L4, NAP1L5, NAPG, NAPSA, NAV2,
NAV3, NBAS, NBEA, NBEAL1, NBEAL2, NBPF1, NCAN, NCCRP1, NCEH1, NCF4, NCK1,
NCKAP1L, NCL, NCOA1, NCOA2, NCOA3, NCOA7, NCOR1, NCOR2, NDFIP1, NDNF,
NDST3, NDUFAB1, NDUFAF8, NDUFB8, NEB, NEBL, NECTIN1, NECTIN4, NEFM,
NEGR1, NEK4, NEK9, NELL1, NEMF, NEPRO, NETO2, NEURL2, NEUROD4, NF1, NF2,
NFASC, NFE2L2, NFKB2, NFKBIA, NFKBIZ, NFRKB, NFYC, NGFR, NGLY1, NHEJ1, NHS,
NHSL2, NID2, NINL, NIPAL3, NKAIN3, NKAP, NKAPL, NKD2, NLRP13, NLRP14, NLRP3,
NLRP6, NLRP7, NMNAT1, NMRAL1, NMT1, NMUR1, NNMT, NOBOX, NOD2, NODAL,
NOL4, NOL8, NOMI, NOP2, NOP58, NOP9, NOS3, NOTCH1, NOTCH2, NOTCH3, NOTCH4,
NOXA1, NPAS2, NPHP3, NPHS1, NPIPB15, NPRL3, NPSR1, NRID2, NR1H2, NR1H3,
NR3C1, NRBF2, NRIP1, NRN1, NRP1, NRSN1, NRXN1, NRXN3, NSD1, NSD2, NSF,
NSMCE1, NSUN2, NSUN5, NT5C, NT5DC1, NTRK1, NUAK1, NUAK2, NUB1, NUCB2,
NUDT16, NUDT17, NUFIP1, NUGGC, NUP155, NUP188, NUP210, NUP210L, NUP214,
NUP98, NUTM2F, NVL, NXF1, OAS1, OAZ1, OBSCN, OC90, OCRL, OCSTAMP, OFD1,
OGDHL, OGFR, OLIG3, OPHN1, OPLAH, OPN3, OR10G7, OR11G2, OR11H12, OR13A1,
OR13G1, OR14C36, OR1B1, OR2L13, OR2M3, OR2T33, OR2T35, OR4C15, OR4D1, OR4D11,
OR4L1, OR52N2, OR5K1, OR5M3, OR5T1, OR6C1, OR6K6, OR6N2, OR6S1, OR7C2, OR8B3,
OR8J1, OR8K5, OR8U1, OR9Q2, ORAI1, ORC1, ORC5, OSBPL1A, OSGIN2, OSMR, OSR2,
OTOF, OTOGL, OXSR1, P3H2, P4HB, PABPC1, PABPC1L, PABPC3, PACS2, PAIP1, PAIP2,
PALB2, PALLD, PALMD, PAN2, PAPD7, PAPOLB, PAPPA2, PARD3, PARD3B, PARD6B,
PARL, PARP10, PARP4, PASD1, PASK, PATJ, PAX5, PAX7, PBRM1, PBX1, PCDH11Y,
PCDHA10, PCDHA12, PCDHA5, PCDHA9, PCDHB12, PCDHB13, PCDHB15, PCDHB6,
PCDHGA2, PCDHGA7, PCDHGB7, PCF11, PCGF1, PCGF2, PCLO, PCM1, PCNX2, PCSK2,
PCSK5, PDAP1, PDCD11, PDCD6IP, PDE12, PDE1C, PDE3B, PDE4B, PDE4DIP, PDGFB,
PDGFD, PDK4, PDP1, PDP2, PDPR, PDS5B, PDSS1, PDSS2, PDZD2, PDZRN3, PEBP1,
PELP1, PER3, PES1, PEX12, PFKL, PGBD4, PHACTR2, PHACTR4, PHF19, PHF23, PHF3,
PHF5A, PHKA1, PHLDA3, PHLDB1, PHLDB2, PHTF1, PHYH, PI4KA, PIAS1, PIAS4,
PIEZO1, PIGV, PIGZ, PIK3C2A, PIK3C2G, PIK3CA, PIK3CB, PIK3CG, PIK3R4, PIM1,
PIWIL2, PIWIL3, PKD1, PKD1L1, PKD1L3, PKHD1L1, PKLR, PKN2, PLCB2, PLCB3,
PLCB4, PLCD3, PLCE1, PLCG2, PLD2, PLD5, PLEC, PLEKHA4, PLEKHA8, PLEKHD1,
PLEKHF2, PLEKHG3, PLEKHG5, PLEKHG6, PLEKHH3, PLEKHJ1, PLEKHM3, PLEKHO2,
PLIN4, PLK2, PLK4, PLPP2, PLPPR1, PLS1, PLSCR2, PLXNB1, PLXNB2, PLXNC1,
PLXND1, PMFBP1, PMPCA, PNLIPRP3, PNRC1, POC1A, POFUT1, POLD3, POLE, POLQ,
POLR2L, POLR3GL, POLR3H, POM121C, POMT2, POMZP3, POP1, POTEE, POTEF,
POTEH, POTEJ, POU2F1, POU3F2, POU3F3, PPARA, PPARG, PPARGC1A, PPAT, PPEF2,
PPID, PPIH, PPM1L, PPM1M, PPP1R12A, PPP1R12B, PPP1R12C, PPP1R13L, PPP1R15B,
PPP1R2P9, PPP1R35, PPP1R3C, PPP2CA, PPP2R1B, PPP2R3B, PPP4R1, PPP4R3CP, PPP4R4,
PPT1, PPWD1, PQLC1, PQLC3, PRAMEF12, PRB2, PRDM10, PRDM15, PRDM16, PRDM2,
PRDM4, PRDM5, PRDM7, PRDM9, PRDX2, PREX2, PRICKLE2, PRIM1, PRKD1, PRKDC,
PRKG2, PRMT6, PROKR2, PROM2, PRPF39, PRPF6, PRPF8, PRR14L, PRR16, PRR7, PRRC1,
PRRC2C, PRRT3, PRRX1, PRSS22, PRSS47, PRTFDC1, PRUNE2, PRX, PSIP1, PSMB8,
PSMB9, PSMC5, PSMD1, PSMD13, PSTK, PTCH1, PTDSS2, PTGES3L, PTGS1, PTH2R,
PTPN11, PTPN13, PTPN22, PTPN23, PTPN3, PTPRB, PTPRC, PTPRF, PTPRH, PTPRO,
PTPRT, PTPRZ1, PTX3, PUM1, PWWP2B, PXK, PXMP2, PYHIN1, PZP, QPCTL, QRFPR,
QRICH2, R3HCC1L, RAB3IL1, RAB44, RABGGTB, RABL3, RAD51AP1, RAD51AP2,
RAD51C, RAF1, RALBP1, RALGAPA2, RALGDS, RALYL, RANBP2, RANBP6, RANBP9,
RANGRF, RAPGEF2, RAPGEF4, RAPGEF5, RAPH1, RARG, RARS, RASAL3, RASIP1,
RASL11A, RASSF4, RB1, RBCK1, RBL1, RBM15, RBM20, RBM26, RBM3, RBM42, RBP4,
RCBTB1, RCC1, RCC2, RCN2, RCOR1, RDH10, RDH11, RDH5, RDX, RECQL5, REELD1,
REG4, RELL2, RELN, REM2, REPIN1, REPS1, REPS2, REV1, REV3L, RFT1, RFX3,
RFXANK, RGL3, RGMB, RGP1, RGS14, RGS22, RGS7, RGSL1, RHBDF2, RHEBL1, RHOA,
RHOB, RHOBTB1, RHOG, RHOT1, RHOV, RIBC2, RICTOR, RIF1, RIMBP2, RIMBP3,
RIPPLY2, RIPPLY3, RIT2, RLIM, RNF115, RNF123, RNF138, RNF14, RNF144B, RNF157,
RNF17, RNF213, RNF215, RNF31, RNF32, RNF40, RNFT2, RNGTT, ROBO2, ROCK1,
ROPN1B, ROS1, RP1, RP2, RPGRIP1L, RPIA, RPL10L, RPL18A, RPRD2, RPS6KA6, RRM1,
RRNAD1, RRP8, RRS1, RSAD1, RSPH9, RTL1, RTN4, RTN4RL1, RTN4RL2, RTTN,
RUNDC1, RUNDC3A, RUNDC3B, RUNX1, RUSC1, RWDD2A, RWDD2B, RXFP2, RXRA,
RYR1, RYR2, RYR3, S100A4, SIPR5, SAAL1, SACS, SALL2, SAMD4A, SAMD9, SAMHD1,
SAR1B, SARDH, SAT2, SATB1, SAXO2, SBF1, SBF2, SBNO1, SBSPON, SCAF1, SCAF8,
SCAMP1, SCAND1, SCAP, SCARB2, SCFD1, SCFD2, SCMH1, SCN10A, SCN1A, SCN3A,
SCN4A, SCN7A, SCP2, SCPEP1, SCRIB, SCRT2, SCYL3, SDCBP, SDCCAG8, SDHAF3,
SDK1, SEC14L1, SEC16B, SEC23B, SEC24B, SEC24C, SECISBP2L, SEL1L3, SELENON,
SEMA3C, SEMA4A, SEMA4F, SEMA5A, SENP5, SERBP1, SERF2, SERPINA10,
SERPINA12, SERPINA9, SERPINB11, SERPINB13, SERPINB2, SERPINB6, SETD1B,
SETDB1, SETX, SEZ6, SF3B1, SF3B2, SFMBT1, SFMBT2, SFTPA1, SFXN1, SFXN4, SGK1,
SGMS1, SGSM3, SH3GL1, SH3PXD2B, SHANK1, SHANK2, SHB, SHCBP1, SHCBP1L,
SHISA9, SHKBP1, SHQ1, SHROOM2, SI, SIAH3, SIGLEC12, SIGLEC6, SIGLEC8, SIK3,
SIN3B, SIPA1L2, SIRPB1, SIRPB2, SIRT1, SIRT2, SIRT3, SIRT7, SIT1, SIX5, SKI, SKIDA1,
SKIL, SKOR2, SLC11A1, SLC12A6, SLC12A7, SLC12A9, SLC13A3, SLC14A1, SLC15A5,
SLC16A4, SLC16A8, SLC17A1, SLC17A7, SLC18B1, SLC19A3, SLC1A6, SLC22A16,
SLC22A18AS, SLC22A2, SLC26A4, SLC26A8, SLC27A2, SLC2A3, SLC30A1, SLC30A10,
SLC30A8, SLC33A1, SLC34A1, SLC35B2, SLC35F2, SLC38A1, SLC38A10, SLC38A2,
SLC38A3, SLC39A6, SLC39A8, SLC3A1, SLC41A2, SLC45A1, SLC4A2, SLC4A5, SLC52A2,
SLC52A3, SLC6A11, SLC6A6, SLC7A7, SLC8A2, SLC9A2, SLC9A4, SLC9A5, SLC9A7,
SLCO1A2, SLCO2A1, SLCO4A1, SLF1, SLF2, SLFNL1, SLIT2, SLITRK2, SLITRK5,
SLITRK6, SLK, SLPI, SLTM, SMAD2, SMAD5, SMAP2, SMARCADI, SMARCC1,
SMARCC2, SMARCD1, SMC1A, SMC5, SMCR8, SMG8, SMIM10, SMOC1, SMOC2, SMPD4,
SMPDL3B, SMS, SMTN, SMURF2, SNAP25, SNRK, SNX13, SNX14, SNX17, SNX20, SNX25,
SNX29, SNX3, SNX33, SNX9, SOAT2, SOCS5, SOCS7, SOHLH2, SOX15, SOX18, SOX2,
SOX30, SOX5, SOX6, SP100, SP140L, SPAG16, SPAG17, SPAG6, SPATA13, SPATA16,
SPATA24, SPATA2L, SPATA3, SPATA31D1, SPATA31E1, SPATA4, SPATA5L1, SPATS1,
SPATS2L, SPDYE3, SPDYE5, SPECC1, SPEN, SPG11, SPHK1, SPICE1, SPIDR, SPIRE1,
SPIRE2, SPOCD1, SPRYD4, SPSB2, SPTA1, SPTAN1, SPTBN4, SQSTM1, SRCAP, SRF,
SRFBP1, SRGAP2, SRM, SRRM2, SRSF2, SRSF6, SRSF7, SRSF9, SSC5D, SSH2, SSX3, ST18,
ST7, ST8SIA1, STAG3, STARD13, STARD5, STARD9, STIL, STK19, STK31, STK35,
STKLD1, STON1, STRAP, STRC, STRIP2, STT3B, STX10, STX18, STXBP5, STXBP5L,
STYK1, SUCO, SULTIA2, SUPT20HL1, SUPT20HL2, SUPT3H, SUPT6H, SUSD2, SUSD4,
SUSD5, SVEP1, SVIL, SWI5, SWT1, SYAP1, SYCE1, SYCP2, SYCP3, SYDE2, SYMPK,
SYNE1, SYNE2, SYNJ1, SYT1, SYT10, SYT11, SYT6, SYT7, SYT8, SZT2, TAB2, TACC1,
TACC2, TACO1, TADA2A, TAF10, TAF1C, TAF1L, TAF4, TAF4B, TAF6, TANGO2, TAOK1,
TARS, TAS2R42, TAS2R5, TATDN2, TBCID1, TBC1D10C, TBC1D19, TBC1D2B,
TBC1D9B, TBCB, TBCK, TBX19, TBX2, TBX21, TBX3, TBX6, TCF3, TCIRGI, TCLIB,
TCOF1, TCP11L2, TCTN2, TDRD1, TDRD6, TDRD9, TEAD4, TECRL, TEK, TEKTI, TENI,
TENM2, TENM4, TEPSIN, TERF2IP, TERT, TESK2, TET1, TET2, TET3, TEX14, TEX33,
TFAP2C, TFR2, TG, TGFBR1, TGIF2LX, TGS1, THADA, THAP1, THAP11, THAP3, THBD,
THBS2, THOC5, THSD7A, THSD7B, THUMPD2, TIAM2, TIE1, TIFA, TIGD2, TIGD6,
TIMELESS, TIMM29, TIMM44, TLDC1, TLE3, TLE4, TLK2, TLL2, TLN2, TLR4, TLR8,
TM7SF3, TMA7, TMC2, TMC5, TMC7, TMCC1, TMED8, TMEM127, TMEM131,
TMEM132B, TMEM139, TMEM144, TMEM151B, TMEM156, TMEM158, TMEM168,
TMEM177, TMEM184C, TMEM200C, TMEM238, TMEM245, TMEM253, TMEM259,
TMEM27, TMEM38B, TMEM57, TMEM61, TMEM63A, TMEM64, TMEM68, TMEM71,
TMF1, TMIGD2, TMOD4, TMPRSS2, TMPRSS7, TMPRSS9, TMTC1, TNFAIP2,
TNFRSF10A, TNFRSF1A, TNIK, TNK2, TNKS, TNKS1BP1, TNRC6A, TNRC6B, TNS3,
TNS4, TOMM34, TONSL, TOP2B, TOP3B, TOPBP1, TP53, TP53BP2, TP53I11, TPGS1,
TPGS2, TPK1, TPO, TPR, TPTE, TRA2A, TRAF3IP1, TRAF3IP3, TRAF5, TRAK1, TRANK1,
TRAPPC8, TREM2, TRERF1, TRHDE, TRIM14, TRIM24, TRIM26, TRIM32, TRIM33,
TRIM35, TRIM37, TRIM39, TRIM47, TRIM50, TRIM51, TRIM58, TRIM60, TRIM66,
TRIM73, TRIM74, TRIO, TRIP11, TRIP6, TRMT12, TRMU, TRNAU1AP, TRNP1, TRPA1,
TRPC1, TRPC4, TRPC5, TRPC7, TRPM1, TRPM6, TRRAP, TSC1, TSC2, TSEN34, TSGA10IP,
TSHZ3, TSKS, TSPAN14, TSSC1, TSSK4, TSTD1, TTC17, TTC21A, TTC21B, TTC24, TTC3,
TTC34, TTC6, TTI1, TTLL11, TTLL3, TTLL5, TTN, TTYH3, TUBA4B, TUBD1, TUBGCP6,
TULP3, TXK, TXNIP, TYMS, TYRO3, TYW1, U2SURP, UACA, UBA1, UBA2, UBA5, UBA6,
UBAP2L, UBC, UBE2E3, UBE2J1, UBE2M, UBE2Z, UBE3A, UBL3, UBN2, UBQLN1,
UBQLNL, UBR4, UBR5, UBR7, UBXN2B, UBXN7, UCHL3, UCP1, UFL1, UGCG, UGDH,
UGGT2, UGT2B15, ULK4, UMODL1, UMPS, UNC13B, UNC13D, UNC5B, UPK3A, URB1,
USH2A, USP1, USP10, USP16, USP17L22, USP21, USP22, USP29, USP31, USP34, USP38,
USP42, USP48, USP6, USP8, USP9X, USP9Y, UTP18, UTRN, UTS2, UTY, UVSSA, VAMP1,
VANGL2, VASP, VAV2, VAV3, VCAN, VCL, VCP, VCPIP1, VEPH1, VIPR2, VNN1, VOPP1,
VPS13B, VPS13D, VPS33A, VPS33B, VPS41, VSTM2B, VWA1, VWA2, VWA3B, VWA5B2,
VWA8, VWCE, VWDE, VWF, WAPL, WASHC4, WASHC5, WDFY3, WDFY4, WDR24,
WDR26, WDR36, WDR48, WDR49, WDR54, WDR59, WDR5B, WDR66, WDR72, WDR74,
WDR78, WDR87, WEE1, WFDC1, WHAMM, WHRN, WIPF3, WNK1, WNK4, WNT8B,
WRNIP1, WTAP, WTIP, WWC2, XIRP2, XPO1, XPO6, XRCC1, XRCC3, XRCC5, XRN2,
XYLB, XYLT1, YARS2, YDJC, YEATS2, YTHDC2, YTHDF2, ZAK, ZBTB11, ZBTB21,
ZBTB37, ZBTB38, ZBTB46, ZBTB48, ZBTB7B, ZBTB9, ZC2HC1C, ZC3H13, ZC3H6,
ZC3HC1, ZCCHC16, ZCCHC3, ZCCHC4, ZDBF2, ZDHHC11, ZDHHC18, ZFAND4, ZFHX3,
ZFHX4, ZFP36L1, ZFYVE1, ZFYVE16, ZFYVE26, ZFYVE27, ZIC4, ZIK1, ZKSCAN5,
ZMYM4, ZMYND12, ZMYND19, ZMYND8, ZNF106, ZNF14, ZNF184, ZNF212, ZNF217,
ZNF219, ZNF224, ZNF236, ZNF239, ZNF256, ZNF263, ZNF276, ZNF280C, ZNF282, ZNF284,
ZNF292, ZNF302, ZNF316, ZNF317, ZNF318, ZNF329, ZNF331, ZNF335, ZNF341, ZNF343,
ZNF365, ZNF394, ZNF395, ZNF397, ZNF407, ZNF408, ZNF418, ZNF419, ZNF420, ZNF436,
ZNF493, ZNF526, ZNF527, ZNF536, ZNF547, ZNF552, ZNF557, ZNF560, ZNF562, ZNF565,
ZNF569, ZNF57, ZNF577, ZNF592, ZNF610, ZNF618, ZNF623, ZNF625, ZNF638, ZNF639,
ZNF644, ZNF660, ZNF678, ZNF7, ZNF703, ZNF704, ZNF710, ZNF711, ZNF724, ZNF76,
ZNF766, ZNF771, ZNF773, ZNF775, ZNF780A, ZNF781, ZNF782, ZNF792, ZNF800,
ZNF804A, ZNF805, ZNF806, ZNF816, ZNF823, ZNF827, ZNF835, ZNF841, ZNF850, ZNF865,
ZNF888, ZNF93, ZNFX1, ZPBP, ZSCAN12, ZSCAN26, ZSCAN30, ZSWIM5, ZSWIM6,
ZXDB, ZXDC, ZYG11A, ZYG11B, ZZZ3

TABLE 8
List of genes with SNVs and InDels of basal urine
samples detected by whole exome sequencing.

A1BG, A2M, A3GALT2, A4GALT, A4GNT, AADACL4, AADAT, AAGAB, AARS, AATK,
ABAT, ABCA1, ABCA12, ABCA13, ABCA4, ABCA6, ABCA8, ABCB11, ABCC10,
ABCC12, ABCC2, ABCC3, ABCC4, ABCC6, ABCC9, ABCD2, ABCD3, ABCE1, ABCF2,
ABCG5, ABHD12B, ABHD17A, ABHD17C, ABHD2, ABHD6, ABI1, ABI3BP, ABL1, ABL2,
ABR, ABRA, ACACA, ACACB, ACAD10, ACADVL, ACAN, ACAP3, ACCSL, ACE,
ACIN1, ACO1, ACOX1, ACSF3, ACSL4, ACSL6, ACSM2B, ACSS1, ACTC1, ACTN1,
ACTR3B, ACTR3C, ACTR6, ACTR8, ACVR1, ACVR2A, ACVRL1, ADAM2, ADAM29,
ADAM30, ADAM33, ADAMDEC1, ADAMTS1, ADAMTS10, ADAMTS12, ADAMTS13,
ADAMTS14, ADAMTS18, ADAMTS19, ADAMTS2, ADAMTS3, ADAMTS5, ADAMTS7,
ADAMTS9, ADAMTSL1, ADAMTSL5, ADAP2, ADAR, ADARB2, ADAT1, ADCY1,
ADCY10, ADCY5, ADCY7, ADCY8, ADD1, ADGB, ADGRB1, ADGRB3, ADGRF3,
ADGRG4, ADGRG6, ADGRG7, ADGRL3, ADGRV1, ADH7, ADRA1A, ADRA1B,
ADRA1D, ADRA2A, AFAP1, AFAP1L2, AFF1, AFF3, AFF4, AFG3L2, AFTPH, AGA,
AGAP11, AGAP3, AGAP4, AGBL1, AGBL3, AGER, AGFG1, AGFG2, AGO3, AGO4,
AGPAT5, AGPS, AGT, AGTPBP1, AHCTF1, AHCYL1, AHNAK, AHNAK2, AIRE, AK2,
AK7, AKAP10, AKAP12, AKAP13, AKAP3, AKAP6, AKAP7, AKAP8, AKAP9, AKNA,
AKR1B1, AKR1C2, AKTIP, ALDH18A1, ALDH1A1, ALDH1A3, ALDH3A2, ALDH5A1,
ALDOA, ALDOB, ALG10, ALG10B, ALK, ALKBH1, ALMS1, ALOX12, ALPP, ALPPL2,
ALX1, ALYREF, AMBP, AMDHD2, AMER1, AMER3, AMN, AMOTL1, AMPD1, AMT,
AMY2B, AMZ2, ANAPC1, ANAPC4, ANAPC7, ANGEL1, ANGEL2, ANHX, ANK2, ANK3,
ANKAR, ANKFN1, ANKRD11, ANKRD17, ANKRD18A, ANKRD20A1, ANKRD20A2,
ANKRD20A3, ANKRD34B, ANKRD35, ANKRD36, ANKRD36B, ANKRD36C, ANKRD45,
ANKRD50, ANKRD55, ANKRD61, ANKRD62, ANKRD63, ANKS1B, ANLN, ANO1,
ANO10, ANO2, ANO4, ANOS1, ANXA2R, ANXA9, AOC3, AOX1, AP1B1, AP1G2, AP2A1,
AP2B1, AP2M1, AP3B2, AP4B1, AP4E1, AP5B1, AP5M1, APBA1, APC, APC2, APH1A,
APOB, APOBEC1, APOBEC3F, APOE, APOPT1, APPBP2, AQP10, AQP8, AQR, ARAP1,
AREL1, ARFGAP3, ARFGEF2, ARFGEF3, ARFIP1, ARG1, ARG2, ARHGAP15,
ARHGAP20, ARHGAP21, ARHGAP23, ARHGAP27, ARHGAP32, ARHGAP35, ARHGAP39,
ARHGAP42, ARHGAP5, ARHGAP6, ARHGEF1, ARHGEF10, ARHGEF12, ARHGEF17,
ARHGEF18, ARHGEF25, ARHGEF40, ARID1A, ARID2, ARID3C, ARID5B, ARL14,
ARMC1, ARMC12, ARMC4, ARMC6, ARMCX4, ARNT, ARNTL, ARPC4, ARRDC2, ARSE,
ARVCF, ASAP1, ASB13, ASB2, ASCC1, ASCC2, ASCC3, ASCL1, ASCL3, ASIC4, ASL,
ASPDH, ASPM, ASTE1, ASTN1, ASTN2, ASXL1, ASXL2, ATAD2, ATAD3C, ATAD5,
ATCAY, ATF2, ATF6B, ATG14, ATG16L1, ATG16L2, ATG2B, ATG9A, ATM, ATMIN,
ATP10A, ATP11B, ATP11C, ATP13A5, ATP1A3, ATPIB1, ATP2A3, ATP2B1, ATP2B2,
ATP2B3, ATP2C1, ATP6V0D2, ATP6V1C2, ATP7A, ATP8A2, ATP8B1, ATP8B2, ATP8B4,
ATP9B, ATR, ATRN, ATRX, ATXN3, ATXN7L1, ATXN7L3B, AUP1, AURKA, AUTS2,
AVL9, AXDND1, AXIN1, AXIN2, AZU1, B3GALT6, B4GALT2, B4GALT3, BACH1,
BACH2, BAD, BAG6, BAIAP2L2, BAMBI, BAP1, BAX, BAZ1A, BAZ2A, BAZ2B, BBS1,
BBS12, BBX, BCAM, BCAR1, BCAR3, BCAS1, BCAT2, BCDIN3D, BCKDHA, BCL2L12,
BCL2L15, BCL6B, BCL7C, BCL9, BCL9L, BCLAF1, BCOR, BCORL1, BCR, BDP1,
BEGAIN, BHMT, BICD1, BICD2, BICDL2, BIN2, BIRC3, BIRC6, BLM, BMP1, BMP10,
BMPR2, BMS1, BNC1, BNC2, BNIP3, BNIP3L, BOD1, BPIFB1, BPTF, BRAF, BRCA1,
BRCA2, BRD1, BRD2, BRD4, BRD7, BRD8, BRF1, BRINP1, BRINP2, BRPF1, BRS3,
BRSK1, BRSK2, BRWD1, BRWD3, BSN, BTBD1, BTBD16, BTBD19, BTF3L4, BTG2,
BTN3A3, BTNL2, BUB3, BUD13, BVES, C10orf11, C10orf82, C11orf45, C11orf65, C11orf80,
C11orf86, C12orf29, C12orf42, C12orf74, C14orf37, C15orf39, C15orf41, C15orf52, C16orf70,
C16orf72, C17orf50, C17orf62, C17orf97, C18orf8, C19orf44, C19orf54, C19orf67, C19orf71,
C1orf101, C1orf105, C1orf106, C1orf185, C1orf189, C1orf198, C1orf234, C1orf43, C1orf56,
C1QL3, C1S, C2, C20orf196, C22orf15, C22orf23, C22orf39, C2CD4D, C2CD5, C2orf16,
C2orf66, C2orf71, C2orf80, C2orf81, C3orf20, C3orf58, C3orf67, C3orf80, C4A, C4orf3,
C4orf51, C5, C5orf22, C5orf42, C5orf60, C6orf141, C6orf222, C6orf226, C6orf89, C7, C7orf25,
C7orf26, C8orf33, C8orf34, C9orf129, C9orf131, C9orf50, C9orf57, CA1, CA14, CA8, CA9,
CACNA1A, CACNA1C, CACNA1E, CACNA1F, CACNA1G, CACNA1H, CACNA1I,
CACNA2D1, CACNA2D2, CACNA2D4, CAD, CADM1, CAGE1, CALCOCO1, CALCOCO2,
CALCR, CALML4, CALR, CALR3, CAMKK2, CAMSAP1, CAMSAP3, CANX, CAPN10,
CAPN13, CAPN14, CAPZA3, CARD11, CARF, CARS2, CASC3, CASK, CASKIN1, CASP10,
CASP3, CASP8, CASS4, CAST, CATSPERB, CBFA2T3, CBFB, CBL, CBLL1, CC2D1A,
CC2D1B, CC2D2B, CCAR1, CCDC105, CCDC110, CCDC112, CCDC117, CCDC122,
CCDC127, CCDC129, CCDC13, CCDC138, CCDC141, CCDC150, CCDC151, CCDC158,
CCDC168, CCDC172, CCDC177, CCDC178, CCDC189, CCDC28B, CCDC33, CCDC39,
CCDC40, CCDC42, CCDC47, CCDC51, CCDC57, CCDC6, CCDC68, CCDC7, CCDC70,
CCDC73, CCDC80, CCDC88A, CCDC88B, CCDC92B, CCDC94, CCER2, CCHCR1, CCL11,
CCL20, CCL3L3, CCNB3, CCNL2, CCNT2, CCPG1, CCT2, CCT5, CCT8, CD109, CD163,
CD163L1, CD164L2, CD300LG, CD33, CD44, CD47, CD84, CDAN1, CDC123, CDC14C,
CDC20, CDC37, CDC40, CDC42, CDC42BPB, CDC42EP1, CDC5L, CDC7, CDCP1, CDH1,
CDH10, CDH11, CDH13, CDH15, CDH2, CDH24, CDH26, CDH4, CDH8, CDH9, CDIP1,
CDK11A, CDK11B, CDK12, CDK13, CDK18, CDK5R2, CDK5RAP3, CDK6, CDKALI,
CDKN1A, CDKN2A, CDR1, CDRT1, CDRT15, CDS1, CDYL, CEACAM18, CEACAM7,
CEBPA, CEBPZ, CECR2, CECR5, CEL, CELA1, CELA2B, CELA3A, CELF2, CELF4,
CELF5, CELSR1, CELSR2, CELSR3, CENPE, CENPF, CENPJ, CENPK, CENPP, CENPT,
CEP104, CEP131, CEP152, CEP162, CEP170, CEP170B, CEP192, CEP250, CEP290, CEP295,
CEP350, CEP57, CEP68, CEP76, CEP95, CER1, CES1, CES4A, CFAP126, CFAP157,
CFAP43, CFAP57, CFAP61, CFAP65, CFAP99, CFH, CFHR4, CFHR5, CFI, CFTR, CHCHD6,
CHD3, CHD4, CHD7, CHD8, CHDH, CHEK2, CHIT1, CHL1, CHMP3, CHMP4B, CHMP6,
CHP1, CHRD, CHRM1, CHRM3, CHRNA2, CHRNB2, CHST1, CHST3, CHST4, CIC, CIITA,
CILP2, CISD3, CITED2, CIZ1, CKAP2L, CKB, CKLF, CLASP2, CLCA4, CLCN1, CLCN2,
CLCN3, CLCN7, CLEC11A, CLEC14A, CLEC18C, CLEC4F, CLIC3, CLIC5, CLIP2, CLK3,
CLPX, CLSPN, CLTCL1, CLUH, CLVS2, CMPK2, CMTM4, CMTM7, CMTM8, CMYA5,
CNIH2, CNNM4, CNOT1, CNOT10, CNOT3, CNOT6, CNPY2, CNST, CNTLN, CNTN1,
CNTN6, CNTNAP3, CNTNAP5, CNTRL, COASY, COBL, COBLL1, COL11A2, COL14A1,
COL16A1, COL17A1, COL19A1, COL20A1, COL22A1, COL24A1, COL27A1, COL4A4,
COL4A5, COL5A3, COL6A2, COL6A3, COL9A3, COLEC12, COPG2, COPS6, CORO2A,
COX20, COX4I2, COX5B, COX7B2, CP, CPD, CPE, CPNE4, CPSF6, CPT1A, CPTP, CPXM1,
CR1L, CR2, CRACR2A, CRAMP1, CRB1, CRBN, CREB3L1, CREBBP, CRELD1, CRELD2,
CRKL, CRLF1, CRLF3, CRLS1, CROT, CRY1, CRY2, CRYBG3, CSFIR, CSF2RB, CSH2,
CSMD1, CSMD2, CSMD3, CSN3, CSNK1A1L, CSNK1E, CSNK1G1, CSPG4, CSRNP2,
CST1, CSTF3, CTAGE4, CTAGE9, CTC1, CTCF, CTCFL, CTH, CTIF, CTNNA1, CTNNB1,
CTNND1, CTNND2, CTNS, CTSA, CTSH, CTTNBP2, CUBN, CUL1, CUL4A, CUL7,
CUZD1, CWC27, CWF19L1, CWF19L2, CWH43, CXCL8, CXCR1, CXXC4, CYB5B,
CYB5R2, CYC1, CYCS, CYFIP1, CYFIP2, CYHR1, CYP11B1, CYP1A2, CYP26A1,
CYP26B1, CYP27B1, CYP2A7, CYP2D6, CYP2F1, CYTH1, DAO, DAPK1, DAPK2, DARS2,
DAXX, DBNL, DBT, DBX2, DCAF11, DCAF15, DCAF4, DCAF4L2, DCAF8, DCBLD1,
DCBLD2, DCDC1, DCHS1, DCHS2, DCLK1, DCST2, DCSTAMP, DCTN1, DCUN1D1,
DDAH1, DDR1, DDX20, DDX27, DDX28, DDX3Y, DDX41, DDX42, DDX50, DDX54,
DEAF1, DEDD, DEF6, DEFB4A, DENND1B, DENND4B, DENND5A, DEPDC1, DEPDC4,
DEPDC5, DEPTOR, DEUP1, DGKG, DGKZ, DHDDS, DHH, DHRS4, DHRS4L1, DHTKD1,
DHX16, DHX57, DHX58, DIAPH2, DIAPH3, DICER1, DIDO1, DIEXF, DIP2B, DIS3, DISC1,
DISP1, DISP3, DIXDC1, DLAT, DLC1, DLEC1, DLG1, DLG3, DLG4, DLGAP1, DLGAP3,
DLGAP4, DLGAP5, DLK1, DLL1, DMBT1, DMD, DMKN, DMRTA2, DMRTB1, DMXL1,
DMXL2, DNAAF1, DNAH1, DNAH10, DNAH11, DNAH12, DNAH14, DNAH17, DNAH2,
DNAH3, DNAH5, DNAH6, DNAH7, DNAH8, DNAI1, DNAI2, DNAJB13, DNAJC1,
DNAJC11, DNAJC13, DNAJC14, DNAJC18, DNAJC21, DNAJC5B, DNAJC6, DNASE1L1,
DNASE1L2, DNER, DNHD1, DNMT3A, DNMT3B, DOC2A, DOCK4, DOCK5, DOCK6,
DOCK7, DOCK8, DOK1, DOK5, DOLK, DONSON, DOPEY2, DOT1L, DPAGT1, DPF2,
DPH2, DPP6, DPP8, DPP9, DPY19L1, DPY19L2, DPY19L3, DPYD, DPYSL3, DPYSL5,
DRD2, DRG2, DSC2, DSCAM, DSCAML1, DSCC1, DSG2, DSG3, DSG4, DSPP, DST,
DSTN, DTNA, DTX4, DUOX1, DUOX2, DUOXA2, DUS3L, DUSP12, DUSP16, DUSP2,
DUSP27, DUSP28, DVL3, DXO, DYNC1H1, DYNC112, DYNC2H1, DYNLT3, DYRK1A,
DZIP1, DZIP1L, DZIP3, E2F1, E2F2, EBF2, EBF3, EBF4, EBNA1BP2, ECHDC2, ECT2L,
EDC4, EDEM3, EEF1A1, EFCAB8, EFHC1, EFL1, EFNA5, EGFLAM, EGLN1, EGR2, EHD3,
EHD4, EHMT1, EID2B, EID3, EIF1, EIF2AK4, EIF2B5, EIF2S3, EIF3A, EIF3M, EIF4A1,
EIF4G3, EIF5A2, ELF3, ELK3, ELK4, ELL3, ELOA, ELOA3D, ELOC, ELOVL2, ELOVL4,
ELP3, EMC9, EML1, EML6, EMSY, ENDOG, ENO1, ENO3, ENO4, ENPEP, ENPP2, ENPP7,
ENTPD1, ENTPD4, EP300, EP400, EPAS1, EPB41, EPB41L2, EPB41L3, EPC1, EPG5,
EPHA1, EPHA3, EPHA8, EPHB1, EPHB6, EPOP, EPPKI, EPS15, EPS8L3, EPX, ERAL1,
ERAS, ERBB2, ERBB3, ERBB4, ERC1, ERC2, ERCC1, ERCC2, ERCC3, ERCC5, ERCC6,
ERCC8, ERG, ERI1, ERI3, ERICH3, ERICH6, ERICH6B, ERLIN1, ERMN, ERN1, ERN2,
ERO1B, ERP27, ERP44, ERRFI1, ESCO1, ESM1, ESPL1, ESR1, ESRP2, ESRRG, ETNK1,
ETV4, ETV5, ETV6, EVA1C, EVC2, EVI5, EVPL, EVX2, EXD3, EX05, EXOC3L4, EXOC4,
EXOC7, EXOG, EXOSC10, EXOSC3, EXOSC8, EXPH5, EXT1, EYA1, EYA4, EZH1, EZR,
F2RL2, F5, F7, FAAP24, FABP3, FAM102B, FAM106A, FAM117B, FAM120A, FAM120B,
FAM120C, FAM124B, FAM126B, FAM129A, FAM131C, FAM133B, FAM134A, FAM135B,
FAM13A, FAM149A, FAM153B, FAM155A, FAM161B, FAM170A, FAM171A1, FAM171B,
FAM174B, FAM177A1, FAM179A, FAM180B, FAM184A, FAM186A, FAM186B,
FAM189A1, FAM208A, FAM208B, FAM214A, FAM221A, FAM227A, FAM228A, FAM3D,
FAM45A, FAM47A, FAM47B, FAM47C, FAM50A, FAM53A, FAM53B, FAM53C, FAM63B,
FAM65A, FAM65B, FAM72A, FAM78B, FAM83C, FAM83G, FAM83H, FAM84B, FAM89A,
FAM8A1, FAM91A1, FAN1, FANCB, FANCC, FANCD2, FANCE, FANCI, FANCM, FARP2,
FARS2, FARSA, FAT1, FAT2, FAT3, FAT4, FBLN1, FBLN7, FBN2, FBN3, FBP2, FBRS,
FBRSL1, FBXL13, FBXL18, FBXL19, FBXL2, FBXL4, FBXL5, FBXL7, FBXO10, FBXO11,
FBXO17, FBXO18, FBXO21, FBXO34, FBXO38, FBXO41, FBXW2, FBXW4, FBXW7,
FCGBP, FCGR2A, FCGR2B, FCGRT, FCHO1, FCRL6, FEM1A, FEM1C, FER1L6, FERMT1,
FFAR3, FGA, FGD1, FGD4, FGD5, FGF12, FGF23, FGF4, FGF5, FGFRIOP, FGFR3, FGR,
FH, FIG4, FIGN, FIP1L1, FITM2, FKBP3, FLCN, FLG, FLG2, FLI1, FLNA, FLNC, FLOT2,
FLRT2, FLT1, FLT3, FLT3LG, FLT4, FMN1, FMN2, FMO1, FN1, FN3KRP, FNBP4,
FNDC3A, FNDC5, FNIP2, FOLH1, FOXA1, FOXD2, FOXH1, FOXI3, FOXK2, FOXL1,
FOXL2, FOXO1, FOXO3, FOXP1, FOXP2, FOXQ1, FRAS1, FREMI, FREM2, FREM3,
FRG1, FRMD1, FRMD4A, FRMD4B, FRMD5, FRMPD2, FRS3, FRYL, FRZB, FSD1L, FSIP2,
FTO, FUCA1, FUT4, FUZ, FXYD3, FYB, FYCO1, FYN, FZD1, FZD10, FZD2, GOS2, G2E3,
GAA, GAB1, GAB3, GABRA2, GABRA4, GAL3ST4, GALC, GALK2, GALNT10,
GALNT14, GALNT4, GALNT5, GALNT6, GALNT9, GAMT, GAP43, GAPVD1, GATA2,
GATA3, GATA5, GATAD2A, GATSL3, GBE1, GBF1, GBP1, GBP3, GCC2, GCDH, GCH1,
GCLC, GCN1, GCNT1, GCNT4, GCSH, GDF11, GDPD2, GFAP, GFPT1, GFRA1, GFRA4,
GGA1, GGCX, GGNBP2, GGT1, GGT5, GGTLC1, GHDC, GIGYF1, GIN1, GIPC1, GIT1,
GJB5, GJD4, GK, GKN1, GLE1, GLG1, GLI1, GLI2, GLIPR1L2, GLMN, GLS2, GLTSCR1L,
GMCL1P1, GMDS, GNA13, GNAI2, GNAL, GNAS, GNG7, GNG8, GNPTAB, GNRH1, GNS,
GOLGA2, GOLGA4, GOLGA6A, GOLGA6B, GOLGA6C, GOLGA6L3, GOLGA7B,
GOLGA8F, GOLGA8K, GOLGA8M, GOLGA8N, GOLGA8Q, GOLGB1, GORASP1, GP1BA,
GP1BB, GPA33, GPAM, GPAT2, GPATCH3, GPATCH8, GPBP1L1, GPC2, GPC3, GPC6,
GPD1L, GPHN, GPNMB, GPR137B, GPR149, GPR156, GPR158, GPR179, GPR25, GPR26,
GPR35, GPR39, GPR68, GPR75, GPR87, GPRC6A, GPS2, GPX8, GRAMD1B, GRAMD4,
GRB10, GREB1L, GRHL1, GRHPR, GRID2, GRIK1, GRIN1, GRIN3B, GRIP2, GRIPAP1,
GRK2, GRK4, GRM3, GRM7, GRN, GSDMB, GSGIL, GSGIL2, GSTA1, GSTA3, GTF2F1,
GTF3C1, GTF3C5, GTPBP10, GUCA1B, GUCY1A2, GUCY1A3, GUCY1B3, GYPA, GYPB,
GYS1, GZMB, H3F3A, H3F3B, H6PD, HACD3, HACL1, HADHA, HAND1, HAO2, HARS,
HAVCR1, HBB, HBP1, HBQ1, HBS1L, HCAR2, HCAR3, HCK, HCN1, HDAC4, HDAC7,
HDHD2, HDLBP, HEATR5A, HEATR5B, HEATR6, HECTD1, HECTD2, HECTD3, HECW1,
HELZ, HEPHL1, HERC1, HERC2, HERC3, HES1, HES3, HFM1, HGC6.3, HGD, HGF,
HGFAC, HGSNAT, HHATL, HHIPL2, HIF1AN, HIGD2B, HIPK1, HIPK3, HIRA, HIRIP3,
HIST1H1C, HIST1H1D, HIST1H2AI, HIST1H2AM, HIST1H2BD, HIST1H2BG, HIST1H2BH,
HIST1H2BL, HIST1H2BN, HIST1H3G, HIST1H3H, HIST1H4B, HIST2H2BE, HIST2H3D,
HIVEP3, HK1, HK2, HKDC1, HLA-C, HLA-DQA1, HLA-DQA2, HLA-DQB2, HLA-DRB1,
HLA-DRB5, HLA-E, HLF, HMCN1, HMCN2, HMMR, HNF1A, HNF4G, HNMT, HNRNPC,
HNRNPCL2, HNRNPM, HNRNPR, HNRNPUL1, HOOK3, HORMAD2, HOXA6, HOXA7,
HOXA9, HOXB1, HOXB13, HOXB8, HOXC9, HOXD4, HPDL, HPS6, HPSE, HRAS, HRC,
HRCT1, HRH4, HRNR, HS2ST1, HS3ST4, HS6ST1, HSCB, HSD17B11, HSD17B14,
HSD17B3, HSD17B8, HSD3B2, HSF1, HSP90AA1, HSP90AB1, HSPA4, HSPA6, HSPA8,
HSPA9, HSPB1, HSPH1, HTRA2, HTT, HUNK, HUWE1, HYDIN, ICE1, ID1, IDH1, IDH2,
IDI1, IDI2, IDO1, IDUA, IFFO2, IFIT1B, IFIT5, IFNAR2, IFNL3, IFT122, IFT22, IFT46,
IFT88, IGF1, IGF2R, IGLL1, IGSF1, IGSF9, IGSF9B, IKBKAP, IKZF2, IL10, IL17RA,
IL17RE, IL1A, IL1R2, IL1RAP, IL1RAPL1, IL20RB, IL22RA2, IL27RA, IL3, IL31RA, IL33,
IL36G, IL3RA, IL411, ILK, IMP3, IMPG1, IMPG2, INCA1, INF2, INHA, INHBA, INHBB,
INO80, INO80B, INPPL1, INS-IGF2, INSM1, INTS1, INTS11, INTS8, INTS9, INTU, INVS,
IP6K2, IPCEF1, IPO11, IPO7, IQCA1L, IQCB1, IQCG, IQCM, IQGAP2, IRAK1, IRF1,
IRF2BP1, IRF2BP2, IRF3, IRF6, IRF9, IRS1, IRS2, ISCA2, ITFG2, ITGA2, ITGA2B, ITGA4,
ITGA6, ITGAV, ITGB1BP1, ITGB4, ITGBL1, ITM2A, ITPK1, ITPKA, ITPR2, ITPR3,
ITPRIP, ITSNI, ITSN2, IVD, IWS1, IZUMO2, JADE1, JAG1, JAKI, JAK2, JAM2, JMJD1C,
JMJD4, JMJD7, JMY, JPH1, JUNB, JUP, KANK1, KANK3, KAT2A, KAT6B, KATNAL2,
KBTBD4, KBTBD6, KCNA3, KCNB1, KCNC2, KCNG2, KCNG3, KCNH6, KCNIP4,
KCNJ10, KCNJ5, KCNK16, KCNK7, KCNK9, KCNMB2, KCNN2, KCNQ4, KCNT2,
KCTD16, KDELR1, KDM2A, KDM3A, KDM3B, KDM5A, KDM6A, KDM6B, KDM7A,
KDR, KHDRBS3, KHSRP, KIAA0100, KIAA0232, KIAA0355, KIAA0368, KIAA0430,
KIAA0513, KIAA0556, KIAA0586, KIAA0825, KIAA0907, KIAA1024, KIAA1109,
KIAA1143, KIAA1147, KIAA1191, KIAA1211, KIAA1217, KIAA1456, KIAA1468,
KIAA1524, KIAA1683, KIAA1841, KIAA2026, KIDINS220, KIF15, KIF1B, KIF20A,
KIF20B, KIF21B, KIF3B, KIF3C, KIF9, KIFAP3, KIFC2, KIFC3, KIN, KIT, KIZ, KLC1,
KLF10, KLF13, KLF18, KLF5, KLHL2, KLHL20, KLHL3, KLHL33, KLHL34, KLHL35,
KLHL41, KLHL6, KLHL7, KLK13, KLK3, KLK9, KLKB1, KLLN, KMT2A, KMT2B,
KMT2D, KNSTRN, KNTC1, KPNA1, KPNA4, KPNA6, KPTN, KRAS, KRBA2, KRIT1,
KRT12, KRT18, KRT19, KRT23, KRT26, KRT27, KRT3, KRT38, KRT40, KRT6B, KRT7,
KRT78, KRT80, KRTAP10-11, KRTAP10-2, KRTAP10-3, KRTAP10-5, KRTAP10-6,
KRTAP10-7, KRTAP1-1, KRTAP1-4, KRTAP5-3, KRTAP5-4, KRTAP9-3, KYNU, LITD1,
L3MBTL1, LACE1, LAD1, LAMA1, LAMA2, LAMA3, LAMB1, LAMB2, LAMC3,
LAMTOR3, LANCL2, LARP4, LARP4B, LARP7, LARS, LARS2, LATS1, LATS2, LBX2,
LCAT, LCE4A, LCN15, LCNL1, LDHA, LDHC, LDLR, LDLRAD2, LDLRAD3, LDLRAD4,
LEF1, LEMD3, LENG1, LGALS9B, LHX8, LHX9, LIFR, LIG4, LILRA2, LILRA6, LILRB5,
LIMCH1, LIN54, LINC00116, LINC00176, LITAF, LMLN, LMNA, LMNTD1, LM04, LMO7,
LMOD1, LMOD2, LMTK2, LNPEP, LNPK, LOC100128108, LOC100129307,
LOC101928841, LOC389602, LOC645177, LOC729159, LOXHD1, LOXL3, LPCAT3,
LPCAT4, LPIN2, LPL, LPP, LRBA, LRFN4, LRFN5, LRGUK, LRIF1, LRIG2, LRIG3, LRP1,
LRP10, LRPIB, LRP2, LRP4, LRP6, LRRC14, LRRC17, LRRC37A2, LRRC3C, LRRC41,
LRRC45, LRRC7, LRRC9, LRRFIP1, LRRK1, LRRK2, LRSAM1, LSS, LTB4R, LTB4R2,
LTBP2, LTBP3, LTBP4, LTBR, LTF, LTK, LTN1, LTVI, LUC7L, LUC7L2, LY6G5B, LY6H,
LY9, LYG1, LYPD3, LYPLA1, LYRM7, LYSMD2, LYST, LZTS2, M1AP, M6PR, MACF1,
MADD, MAEA, MAGEB6, MAGEC1, MAGEF1, MAGEL2, MAGI3, MALT1, MAMDC2,
MAML1, MAML2, MAML3, MAN1C1, MANBAL, MAOA, MAP2, MAP2K1, MAP3K10,
MAP3K13, MAP3K14, MAP3K21, MAP3K5, MAP4, MAP4K1, MAP4K3, MAPK1, MAPK4,
MAPRE2, MAPT, MARCHF9, MARCKS, MARCKSL1, MARCOL, MARK1, MARK2,
MASP1, MASP2, MAST1, MAST2, MAST4, MAT2A, MAT2B, MAZ, MB21D2, MBD1,
MBD2, MBD6, MBLAC2, MBTPS1, MC5R, MCC, MCCC2, MCCD1, MCF2L, MCF2L2,
MCM3, MCM6, MCOLN1, MCTP2, MCUR1, MDC1, MDGA2, MDH1B, MDM4, MDN1,
MECR, MED12, MED13, MED13L, MED18, MED23, MED24, MED25, MED28, MEF2B,
MEGF10, MEGF8, MELK, MELTF, MEPCE, MERTK, MESDC1, MET, METAP2, METTL16,
METTL22, METTL26, METTL3, MEX3D, MFAP1, MFHAS1, MFSD12, MFSD13A,
MFSD14A, MFSD14B, MFSD6, MGA, MGAM, MGAM2, MGAT2, MGAT5B, MGST1,
MIA2, MIA3, MIB2, MICAL1, MICAL2, MICALL1, MICALL2, MIF, MIGA2, MINDY4B,
MINK1, MIOX, MIPOL1, MITF, MKLN1, MLF1, MLH3, MLNR, MLST8, MMAA,
MMACHC, MMP1, MMP19, MMP25, MMP7, MMRN1, MMS19, MMS22L, MN1, MNDA,
MNS1, MOK, MON1A, MORC3, MORF4L1, MPDZ, MPHOSPH10, MPHOSPH8, MPO,
MPP2, MPP3, MPRIP, MRAS, MRC2, MRE11, MRFAP1L1, MROH2B, MROH7, MRPL15,
MRPL21, MRPL3, MRPL38, MRPS27, MSANTD2, MSC, MSH3, MSL3, MST1, MSTIL,
MST1R, MSX2, MT1M, MTA1, MTA2, MTARC1, MTBP, MTFP1, MTHFD1L, MTMR4,
MTOR, MTR, MTSS1L, MTUS1, MTX2, MUC12, MUC16, MUC17, MUC19, MUC2,
MUC21, MUC22, MUC3A, MUC4, MUC5AC, MUC5B, MUC6, MUM1, MUSK, MUSTN1,
MUTYH, MYADM, MYB, MYBPC1, MYBPHL, MYCBP2, MYCL, MYD88, MYDGF,
MYEF2, MYH1, MYH10, MYH2, MYH6, MYH7, MYH7B, MYH9, MYL1, MYLK, MYLK2,
MYO10, MYO15A, MYO15B, MYO16, MYO18B, MYO19, MYO1G, MYO3A, MYO3B,
MYO5C, MYO6, MYO7A, MYO7B, MYO9A, MYOC, MYOF, MYOM2, MYOM3, MYRIP,
MYSM1, MYT1L, N4BP2, NAALADL1, NAALADL2, NAB2, NABP1, NANS, NAP1L2,
NAP1L5, NAPB, NAPEPLD, NAPSA, NARF, NAV3, NBAS, NBDY, NBEA, NBEAL1,
NBEAL2, NBN, NBPF1, NBPF10, NBPF11, NBPF3, NBPF4, NBPF7, NBPF9, NCALD,
NCAM2, NCAN, NCBP1, NCCRP1, NCEH1, NCF4, NCK1, NCKAP1L, NCKAP5L, NCL,
NCOA1, NCOA2, NCOA3, NCOR1, NCOR2, NDFIP1, NDST2, NDST3, NDUFA2,
NDUFAB1, NDUFAF7, NDUFAF8, NDUFB8, NDUFV2, NEB, NEBL, NECTIN1, NEDD4L,
NEFM, NEGR1, NEK4, NEK9, NELL1, NEMF, NES, NET1, NETO2, NEU1, NEU4,
NEURL1, NEURL2, NEUROD4, NF1, NF2, NFASC, NFAT5, NFE2L2, NFE2L3, NFIA, NFIB,
NFKB2, NFKBIA, NFKBIB, NFKBID, NFKBIL1, NFKBIZ, NFRKB, NFU1, NFYC, NGLY1,
NHEJ1, NHLRC1, NHS, NHSL2, NID2, NINL, NIPAL4, NIPSNAP1, NISCH, NKAIN3,
NKAP, NKAPL, NKD2, NKX3-2, NLRC4, NLRP11, NLRP13, NLRP14, NLRP3, NLRP4,
NLRP6, NLRP7, NME7, NME8, NMNAT1, NMRAL1, NMT1, NNMT, NNT, NOBOX, NOD2,
NODAL, NOL4, NOL8, NOM1, NOMO2, NOP2, NOP58, NOP9, NOS1, NOS3, NOTCH1,
NOTCH2, NOTCH2NL, NOTCH3, NOTCH4, NOX4, NPAS2, NPBWR1, NPC1, NPDC1,
NPEPPS, NPFFR2, NPHP3, NPIPB11, NPIPB15, NPR1, NPR2, NPRL2, NPRL3, NR1D2,
NR1H2, NR1H3, NR3C1, NR6A1, NRBF2, NRG1, NRIP1, NRN1, NRP1, NRSN1, NRXN1,
NRXN2, NSD1, NSD2, NSD3, NSF, NSMCE1, NSUN2, NSUN5, NSUN7, NT5C1B, NT5DC1,
NT5DC3, NTRK1, NTRK2, NUAK1, NUAK2, NUCB2, NUDT16, NUFIP1, NUMB, NUP153,
NUP155, NUP188, NUP210, NUP214, NUP98, NUTMI, NUTM2E, NVL, OAS1, OAZ1,
OBSCN, OC90, OCRL, OCSTAMP, OFD1, OGDHL, OGFRL1, OLFM1, OLIG3, OLR1,
OMG, OPA3, OPHNI, OPLAH, OPRD1, OPRK1, OR10G7, OR10G9, OR10J3, OR10V1,
OR11G2, OR11H1, OR11H12, OR11H2, OR14C36, OR1B1, OR1F12, OR1S1, OR1S2,
OR2K2, OR2L13, OR2L3, OR2L8, OR2M3, OR2T35, OR2T4, OR2T8, OR4C15, OR4D11,
OR4F17, OR4F5, OR4L1, OR4M2, OR4N4, OR52J3, OR52N2, OR5AC2, OR5AR1, OR5AS1,
OR5D18, OR5H2, OR5K1, OR5L2, OR5T1, OR6C1, OR6C6, OR6N2, OR6S1, OR8B3,
OR8D4, OR8G2, OR8G5, OR8J1, OR8U1, OR9K2, OR9Q1, ORAI1, ORC1, ORC5, ORC6,
ORM1, OS9, OSBPL1A, OSBPL2, OSBPL3, OSCP1, OSGIN2, OSMR, OSR2, OTOA, OTOF,
OTOG, OTOGL, OVGP1, P3H2, PABPC1, PABPC1L, PABPC3, PACS2, PADI6, PAIP1,
PAIP2, PALB2, PALD1, PALLD, PALM, PALMD, PAM, PAMR1, PAN2, PAPD7, PAPOLB,
PAPPA, PAPPA2, PAPSS2, PARD3B, PARD6B, PARK2, PARL, PARN, PARP1, PARP10,
PARP11, PARP2, PARP4, PARP8, PARP9, PASD1, PASK, PATJ, PAX7, PBRM1, PBX1,
PCDH1, PCDH11Y, PCDH17, PCDH8, PCDH9, PCDHA11, PCDHA3, PCDHA5, PCDHA7,
PCDHA9, PCDHAC1, PCDHB1, PCDHB13, PCDHB15, PCDHB16, PCDHB6, PCDHB8,
PCDHB9, PCDHGA1, PCDHGA2, PCDHGA7, PCDHGA9, PCDHGB7, PCDHGC3, PCED1A,
PCF11, PCGF1, PCGF2, PCGF6, PCLO, PCM1, PCNX2, PCOLCE2, PCSK2, PCSK5, PCSK7,
PCSK9, PCYOX1, PCYT2, PDCD2, PDCD2L, PDE12, PDE3B, PDE4B, PDE4DIP, PDE7B,
PDGFB, PDGFD, PDGFRA, PDGFRB, PDHX, PDIA3, PDK4, PDP2, PDS5B, PDSS1, PDSS2,
PDX1, PDXK, PDZD7, PDZD8, PDZRN3, PEBP1, PELP1, PER3, PES1, PEX3, PFKFB4,
PFKL, PGBD4, PGM2L1, PGM5, PGPEP1, PGR, PGS1, PHACTR2, PHACTR4, PHB2,
PHF13, PHF19, PHF3, PHF5A, PHF6, PHKA1, PHKA2, PHLDA3, PHLDB1, PHLDB3,
PHLPP1, PHRF1, PHTF1, PHYH, PI4KA, PIAS4, PIDD1, PIEZO1, PIEZO2, PIGS, PIGV,
PIK3C2B, PIK3C2G, PIK3CA, PIK3CB, PIK3CD, PIK3CG, PIK3R5, PIM1, PIM3, PINK1,
PIP5K1B, PIWIL1, PIWIL2, PIWIL3, PKD1, PKD1L1, PKD2, PKD2L1, PKHD1, PKHD1L1,
PKLR, PKN2, PLA2R1, PLBD2, PLCB2, PLCB3, PLCD3, PLCE1, PLCG1, PLCG2, PLD1,
PLD2, PLEC, PLEKHA5, PLEKHA7, PLEKHA8, PLEKHD1, PLEKHF2, PLEKHG3,
PLEKHG5, PLEKHG6, PLEKHH3, PLEKHJ1, PLEKHM2, PLEKHM3, PLEKHN1,
PLEKHO2, PLEKHS1, PLIN4, PLK4, PLOD2, PLOD3, PLPP1, PLPP2, PLPP5, PLPPR1,
PLPPR3, PLS1, PLXDC1, PLXNA1, PLXNA2, PLXNB1, PLXNB2, PLXNB3, PMFBP1,
PMPCA, PMS1, PNLIPRP3, PNMA3, PNN, PNPT1, PNRC1, POC1A, POFUT1, POGZ,
POLD1, POLE, POLQ, POLR2F, POLR2L, POLR3F, POLR3H, POM121, POM121C,
POMGNT2, POMT2, POMZP3, POP1, POR, POTEA, POTEC, POTED, POTEE, POTEF,
POTEG, POTEH, POTEJ, POTEM, POU2F1, POU3F1, POU3F2, POU3F3, POU4F3, PPA1,
PPARA, PPARG, PPEF2, PPIAL4D, PPID, PPIH, PPIP5K2, PPM1M, PPP1R12B, PPP1R12C,
PPP1R15B, PPP1R2P9, PPP1R35, PPP1R37, PPPIR3C, PPP2CA, PPP2R1B, PPP2R3B,
PPP2R5C, PPP4R1, PPP4R2, PPP4R3CP, PPP4R4, PPP6R3, PPT1, PPWD1, PQLC1, PQLC3,
PRAMEF1, PRAMEF10, PRAMEF11, PRAMEF12, PRAMEF14, PRAMEF17, PRAMEF2,
PRAMEF33P, PRAMEF5, PRAMEF7, PRB2, PRB4, PRDM1, PRDM10, PRDM2, PRDM4,
PRDM5, PREX2, PRH2, PRIM1, PRKCE, PRKCG, PRKCH, PRKCI, PRKD1, PRKDC,
PRKG2, PRMT6, PROM2, PROSER1, PRPF6, PRPF8, PRPS1L1, PRR14L, PRR15, PRR29,
PRR36, PRR7, PRRC1, PRRC2A, PRRC2B, PRRC2C, PRRT2, PRRT3, PRSS16, PRSS22,
PRSS47, PRSS58, PRTFDC1, PRUNE1, PRUNE2, PRX, PSD3, PSG2, PSG4, PSIP1, PSMA5,
PSMA6, PSMB2, PSMB8, PSMB9, PSMC3, PSMC5, PSMD1, PSMD13, PSMD3, PSORS1C1,
PSTK, PSTPIP1, PTCH1, PTCHD4, PTDSS1, PTDSS2, PTGDR, PTGR2, PTGS1, PTH1R,
PTPN11, PTPN13, PTPN23, PTPN3, PTPRB, PTPRC, PTPRD, PTPRF, PTPRG, PTPRM,
PTPRN, PTPRN2, PTPRQ, PTPRS, PTPRT, PTRF, PUM1, PWWP2B, PXDN, PXDNL,
PXMP2, PXT1, PYHIN1, PYURF, PZP, QKI, QRFPR, QRICH2, QSER1, QSOX1,
RAB11FIP4, RAB11FIP5, RAB15, RAB39B, RAB3C, RAB3GAP2, RAB3IL1, RAB3IP,
RAB44, RAB6B, RABEP2, RABGAP1, RABGAP1L, RABGGTB, RAD21, RAD50,
RAD51AP1, RAD51AP2, RAD51B, RAD51C, RAD52, RAD54L2, RAF1, RALGAPA2,
RALGDS, RALGPS2, RAN, RANBP2, RANBP6, RANBP9, RANGRF, RAPGEF2, RAPGEF4,
RAPGEF5, RAPH1, RARG, RASA2, RASA3, RASA4, RASA4B, RASAL3, RASIP1, RASL12,
RASSF10, RB1, RBBP5, RBBP8, RBCK1, RBFOX1, RBL1, RBL2, RBM14, RBM15, RBM3,
RBM46, RBP4, RBPJL, RC3H1, RCAN2, RCBTB1, RCC2, RCN2, RCOR1, RDH10, RDH11,
RDH12, RDH14, RDH5, RDX, RECQL, REELD1, REG4, RELN, REM2, REPIN1, REPS1,
REPS2, REV3L, REXO4, RFT1, RFWD3, RFX3, RGL1, RGMB, RGP1, RGPD3, RGPD4,
RGS14, RGS22, RGS3, RGS7, RGS7BP, RGSL1, RHBDF1, RHEBL1, RHOA, RHOB,
RHOBTB2, RHOG, RHOQ, RHOT1, RHPN1, RIBC2, RIC1, RICTOR, RIMBP2, RIMBP3,
RIMBP3B, RIMS1, RIN3, RIT2, RLIM, RMND1, RNASE8, RNASEH2B, RNF111, RNF115,
RNF138, RNF14, RNF144B, RNF157, RNF17, RNF213, RNF215, RNF38, RNF43, RNFT2,
ROBO2, ROBO3, ROCK, ROCK2, ROPN1B, ROR1, RORA, ROS1, RP1, RP1L1, RP2,
RPA1, RPGRIP1, RPH3A, RPIA, RPL10L, RPLP0, RPN2, RPP25L, RPP38, RPRD2, RPS2,
RPS20, RPS23, RPS3A, RPS6KA6, RPS9, RPSA, RPTN, RRAGB, RRBP1, RRM1, RRNAD1,
RRP7A, RRS1, RSAD1, RSAD2, RSBN1L, RSC1A1, RSPH1, RSPH10B2, RSPH9, RSRC1,
RTEL1, RTF1, RTL1, RTN4, RTN4IP1, RTN4RL1, RTN4RL2, RTTN, RUNDC1, RUNDC3A,
RUNDC3B, RUNX1, RUSC1, RWDD2A, RXFP2, RXRA, RYR1, RYR2, RYR3, S100A4,
S1PR5, SAAL1, SACS, SALL1, SALL2, SAMD1, SAMD10, SAMD4A, SAMD7, SAMD9,
SAMD9L, SAPCD2, SAR1B, SART1, SAT2, SATB1, SAXO2, SBF1, SBF2, SBNO1, SC5D,
SCAF1, SCAF4, SCAF8, SCAND1, SCAP, SCARB2, SCFD1, SCFD2, SCG3, SCMH1,
SCML2, SCN10A, SCN4A, SCN9A, SCRIB, SCRT2, SCYL3, SDCBP, SDCBP2, SDCCAG8,
SDHA, SDK1, SDPR, SEC14L1, SEC14L4, SEC14L5, SEC23B, SEC24B, SEC24C, SEL1L3,
SELENBP1, SELENON, SELPLG, SEMA4A, SEMA4F, SEMA5A, SEMA5B, SEMA6C,
SEMG2, SENP2, SENP5, SENP7, SERBP1, SERF2, SERINC2, SERPINA10, SERPINA12,
SERPINA9, SERPINB11, SERPINB2, SERPINB6, SERPINF2, SESN2, SET, SETD1A,
SETD1B, SETD2, SETD5, SETDB1, SETX, SEZ6, SF3B1, SFMBT1, SFMBT2, SFTPA1,
SFXN4, SGIP1, SGK1, SGMS1, SGSM3, SH2D2A, SH2D5, SH3GL1, SH3PXD2A, SH3RF1,
SH3TC2, SHANK1, SHANK2, SHB, SHC3, SHCBP1L, SHF, SHH, SHISA9, SHKBP1,
SHOC2, SHOX2, SHQ1, SHROOM2, SI, SIAH2, SIAH3, SIGLEC11, SIGLEC12, SIGLEC6,
SIGMAR1, SIK3, SIM2, SIN3B, SIPA1L2, SIPA1L3, SIRPB1, SIRPB2, SIRT2, SIRT7, SIT1,
SIX5, SKIL, SKIV2L, SKIV2L2, SKOR2, SLC10A4, SLC10A5, SLC12A5, SLC12A6,
SLC12A7, SLC12A9, SLC13A1, SLC13A3, SLC15A3, SLC16A7, SLC16A8, SLC17A1,
SLC18B1, SLC1A6, SLC22A16, SLC22A18AS, SLC24A3, SLC25A11, SLC25A12, SLC26A1,
SLC26A4, SLC26A5, SLC26A7, SLC26A9, SLC27A2, SLC27A4, SLC2A2, SLC2A3,
SLC2A4, SLC30A1, SLC30A5, SLC30A8, SLC33A1, SLC35A3, SLC35B2, SLC35F1,
SLC35F2, SLC35G6, SLC38A1, SLC38A10, SLC38A2, SLC38A3, SLC39A6, SLC39A8,
SLC3A1, SLC43A2, SLC44A3, SLC45A1, SLC45A3, SLC4A2, SLC4A5, SLC52A2,
SLC52A3, SLC5A12, SLC6A11, SLC6A4, SLC6A6, SLC6A7, SLC6A9, SLC7A14, SLC7A7,
SLC8A2, SLC8A3, SLC9A2, SLC9A4, SLC9A5, SLC9B1, SLC9B2, SLCO2A1, SLCO4A1,
SLCO5A1, SLF1, SLF2, SLFN11, SLFN13, SLFNL1, SLITRK2, SLITRK5, SLITRK6, SLK,
SLPI, SLTM, SLURP1, SLX4, SMAD5, SMAP1, SMAP2, SMARCA2, SMARCA4,
SMARCAD1, SMARCAL1, SMARCC1, SMARCC2, SMARCD1, SMC1A, SMC1B, SMC5,
SMCR8, SMIM10, SMIM5, SMOC2, SMOX, SMPD4, SMS, SMTN, SNAP47, SNCAIP,
SNRK, SNRNP35, SNX13, SNX14, SNX17, SNX18, SNX20, SNX25, SNX29, SNX31,
SNX33, SNX5, SNX9, SOAT2, SOHLH2, SORBS1, SORD, SORL1, SOWAHB, SOX1,
SOX15, SOX18, SOX2, SOX30, SOX4, SOX6, SOX9, SP1, SP100, SP140L, SP5, SPACA6,
SPAG17, SPAG5, SPARCL1, SPATA16, SPATA2L, SPATA3, SPATA31C1, SPATA31C2,
SPATA31D1, SPATA32, SPATA33, SPATA4, SPATA5L1, SPATA7, SPATS1, SPATS2L,
SPDYE16, SPDYE3, SPDYE5, SPECC1, SPEF1, SPEG, SPEMI, SPEN, SPG11, SPHK1,
SPICE1, SPIDR, SPINK5, SPIRE1, SPIRE2, SPO11, SPOCD1, SPRED3, SPRN, SPRR3,
SPRYD4, SPTA1, SPTAN1, SPTBN4, SPTBN5, SQLE, SRCAP, SREBF1, SRFBP1, SRGAP1,
SRGAP2C, SRP68, SRRM2, SRRM5, SRSF2, SRSF6, SRSF9, SRY, SSC4D, SSC5D, SSH2,
SSX3, ST18, ST3GAL6, ST6GALNAC5, ST7, ST8SIA1, STAB2, STAG1, STAG2, STAG3,
STAP2, STARD5, STARD9, STAT2, STAT4, STAT6, STC2, STEAP3, STIM2, STK11IP,
STK19, STK31, STK32B, STK35, STON1, STPG2, STRA8, STRADB, STRAP, STRC,
STRIP2, STT3B, STX16, STX18, STXBP5, STXBP5L, STYK1, SUCO, SULT1A2, SULT6B1,
SUMF1, SUPT20HL2, SUPT3H, SUPT6H, SURF6, SUSD2, SUSD5, SVEP1, SVIL, SWTI,
SYCE1, SYCE1L, SYCP2, SYCP2L, SYCP3, SYDE2, SYK, SYMPK, SYNDIG1L, SYNE1,
SYNE2, SYNE4, SYNJ1, SYNM, SYNPO2L, SYT1, SYT10, SYT11, SYT3, SYT6, SYT7,
SYT8, SYTL5, SZRD1, SZT2, TAB2, TACC1, TACC2, TACC3, TAF10, TAF15, TAF1C,
TAF1L, TAF4, TAF4B, TAF6, TAF8, TANC1, TANK, TAOK1, TAS1R3, TAS2R5, TAS2R7,
TATDN2, TAX1BP1, TBC1D10C, TBC1D19, TBC1D2B, TBC1D3, TBC1D31, TBC1D4,
TBC1D9, TBC1D9B, TBCB, TBCCD1, TBCK, TBKBP1, TBX19, TBX2, TBX21, TBX3,
TBX6, TCEA1, TCEA2, TCF25, TCF3, TCF7L2, TCHH, TCIRG1, TCL1B, TCN2, TCOF1,
TCP11L2, TCTEX1D4, TCTN2, TDG, TDRD1, TDRD6, TDRD9, TEAD4, TECRL, TECTB,
TEK, TEKT1, TEN1, TENM1, TENM3, TEPP, TEPSIN, TERF2IP, TERT, TESK1, TESK2,
TET1, TET2, TET3, TEX14, TEX29, TEX33, TEX44, TFAP2A, TFAP2B, TFAP2C, TFAP2D,
TFEB, TFF1, TFPI, TFR2, TFRC, TG, TGFB1, TGFBR3, TGIF1, TGIF2LX, TGM2, TGM4,
TGOLN2, TGS1, THADA, THAP11, THAP12, THAP4, THBD, THBS2, THNSL1, THOC5,
THSD1, THSD7B, TIAL1, TIFA, TIGD2, TIGD6, TIMD4, TIMELESS, TIMM29, TIMM44,
TINAG, TLE3, TLE4, TLK2, TLL2, TLN2, TLR4, TLR8, TM7SF3, TM9SF2, TMA7,
TMBIM4, TMBIM6, TMC2, TMC4, TMC5, TMC7, TMCC1, TMCC2, TMCOSA, TMCO6,
TMED8, TMEM125, TMEM127, TMEM143, TMEM144, TMEM151B, TMEM168,
TMEM177, TMEM178B, TMEM181, TMEM184C, TMEM200B, TMEM200C, TMEM217,
TMEM238, TMEM25, TMEM253, TMEM259, TMEM261, TMEM27, TMEM38B,
TMEM41A, TMEM44, TMEM57, TMEM63A, TMEM67, TMEM68, TMEM80, TMEM87B,
TMPRSS11A, TMPRSS13, TMPRSS15, TMPRSS2, TMPRSS7, TMPRSS9, TMTC1, TMTC2,
TNC, TNFAIP2, TNFRSF10A, TNFRSF19, TNFRSF1A, TNFSF8, TNIK, TNIP3, TNK1,
TNKS, TNKS1BP1, TNN, TNNI3K, TNP2, TNPO2, TNPO3, TNR, TNRC6A, TNRC6B, TNS1,
TNS3, TNS4, TNXB, TOM1, TOM1L1, TOMM20, TOMM22, TOMM34, TONSL, TOP2A,
TOPBP1, TOR3A, TP53, TP53BP1, TP53BP2, TP53I11, TP53113, TPGS1, TPGS2, TPK1,
TPO, TPR, TPRN, TPSAB1, TPSB2, TPSD1, TPTE, TPX2, TRA2A, TRAF3IP1, TRAF5,
TRAK1, TRANK1, TRAPPC8, TREM2, TRERF1, TRIB1, TRIM16, TRIM16L, TRIM24,
TRIM26, TRIM32, TRIM33, TRIM34, TRIM37, TRIM4, TRIM43B, TRIM47, TRIM50,
TRIM51, TRIM58, TRIM59, TRIM60, TRIM63, TRIM64B, TRIM64C, TRIM67, TRIM71,
TRIO, TRIOBP, TRIP11, TRIP6, TRMT1, TRMT12, TRMT1L, TRMT44, TRNP1, TRPA1,
TRPC1, TRPC4, TRPC4AP, TRPC5, TRPMI, TRPM3, TRPM4, TRPM6, TRPS1, TRRAP,
TSC1, TSC2, TSGA10IP, TSHR, TSHZ1, TSHZ2, TSKS, TSNARE1, TSPY8, TSSK4, TSTD1,
TTC1, TTC17, TTC24, TTC27, TTC28, TTC3, TTC30A, TTC31, TTC34, TTC6, TTC7A,
TTF2, TTI1, TTI2, TTK, TTLL11, TTLL3, TTLL5, TTLL7, TTN, TTYH3, TUB, TUBA4B,
TUBB2A, TUBD1, TUBG1, TUBGCP5, TUBGCP6, TUFM, TULP3, TULP4, TXLNA,
TXNDC2, TXNIP, TXNL4B, TYRO3, TYSND1, U2AF1, UACA, UBA5, UBA6, UBAP1,
UBAP2L, UBASH3A, UBC, UBE2E3, UBE2M, UBE20, UBE2Z, UBE4A, UBL7, UBQLN1,
UBR1, UBR3, UBR4, UBR5, UBTFL1, UBXN4, UBXN7, UCHL3, UCK2, UCP1, UFL1,
UGCG, UGDH, UGGT2, UGT2B10, UGT2B4, ULK4, UMODL1, UMPS, UNC13A, UNC13B,
UNC13D, UNC45B, UNC5B, UNC5D, UNC80, UNKL, UPF2, UPF3A, UPK3A, USH1C,
USH2A, USO1, USP1, USP10, USP14, USP17L22, USP20, USP21, USP22, USP29, USP31,
USP34, USP36, USP38, USP42, USP48, USP6, USP8, USP9Y, UTF1, UTP14C, UTRN, UTY,
UVRAG, UVSSA, VAMP1, VANGL2, VARS, VASN, VASP, VAV2, VCAN, VCL, VCP,
VCPIP1, VCX, VCX3B, VEPH1, VEZT, VNN1, VOPP1, VPS11, VPS13B, VPS13D, VPS33B,
VPS52, VSTM1, VSX1, VTCN1, VWA1, VWA2, VWASA, VWA5B2, VWDE, VWF, WAPL,
WARS, WASF1, WASHC1, WASHC2A, WASHC2C, WASHC5, WBP11, WBP2NL,
WBSCR17, WDFY3, WDFY4, WDR13, WDR17, WDR19, WDR24, WDR36, WDR37,
WDR47, WDR48, WDR49, WDR54, WDR5B, WDR63, WDR66, WDR70, WDR72,
WDR83OS, WDR90, WDR93, WEE1, WHAMM, WHRN, WIPF3, WNT11, WNT2B, WNT8B,
WRNIP1, WSB1, WTAP, WTIP, WWC2, WWP2, XDH, XIRP2, XPC, XPO1, XPO4, XRCC3,
XRCC5, XRN2, XYLB, XYLT1, XYLT2, YARS2, YBX2, YDJC, YEATS2, YIPF4, YKT6,
YTHDC2, YTHDF1, YTHDF2, YTHDF3, ZAK, ZAN, ZAP70, ZBTB1, ZBTB11, ZBTB18,
ZBTB37, ZBTB38, ZBTB45, ZBTB46, ZBTB48, ZBTB49, ZBTB7B, ZBTB9, ZC2HC1A,
ZC2HC1C, ZC3H12D, ZC3H14, ZC3H4, ZC3H6, ZC3H7B, ZCCHC14, ZCCHC16, ZCCHC3,
ZDBF2, ZDHHC11B, ZDHHC16, ZDHHC6, ZDHHC7, ZDHHC8, ZEB2, ZFHX3, ZFHX4,
ZFP36L1, ZFP69B, ZFPM2, ZFR, ZFYVE1, ZFYVE16, ZFYVE26, ZFYVE27, ZGLP1, ZIK1,
ZMIZ1, ZMYM4, ZMYND12, ZMYND19, ZMYND8, ZNF106, ZNF12, ZNF131, ZNF154,
ZNF160, ZNF17, ZNF180, ZNF184, ZNF189, ZNF197, ZNF207, ZNF212, ZNF217, ZNF219,
ZNF221, ZNF236, ZNF251, ZNF256, ZNF263, ZNF276, ZNF280C, ZNF281, ZNF282,
ZNF284, ZNF292, ZNF302, ZNF316, ZNF318, ZNF331, ZNF335, ZNF33A, ZNF341, ZNF365,
ZNF391, ZNF394, ZNF397, ZNF398, ZNF407, ZNF416, ZNF419, ZNF420, ZNF423, ZNF429,
ZNF436, ZNF441, ZNF446, ZNF451, ZNF469, ZNF474, ZNF487, ZNF493, ZNF517, ZNF525,
ZNF526, ZNF528, ZNF530, ZNF536, ZNF543, ZNF547, ZNF551, ZNF552, ZNF557, ZNF560,
ZNF565, ZNF577, ZNF578, ZNF580, ZNF587, ZNF592, ZNF595, ZNF606, ZNF609, ZNF610,
ZNF613, ZNF618, ZNF623, ZNF625, ZNF638, ZNF639, ZNF644, ZNF658, ZNF669, ZNF671,
ZNF678, ZNF680, ZNF683, ZNF7, ZNF700, ZNF703, ZNF704, ZNF705E, ZNF710, ZNF76,
ZNF765, ZNF766, ZNF77, ZNF771, ZNF773, ZNF775, ZNF780A, ZNF780B, ZNF782,
ZNF800, ZNF804A, ZNF804B, ZNF806, ZNF808, ZNF816, ZNF823, ZNF827, ZNF831,
ZNF835, ZNF839, ZNF841, ZNF850, ZNF880, ZNF888, ZNF90, ZNF93, ZNF98, ZNFX1,
ZNHIT1, ZNHIT2, ZRSR2, ZSCAN1, ZSCAN12, ZSCAN26, ZSCAN30, ZSCAN32, ZSWIM5,
ZSWIM6, ZWILCH, ZXDC, ZYG11A, ZZZ3

TABLE 9
List of genes with SNVs and InDels detected by personalized MRD panel sequencing.

ABCA4, ABCB5, ABCC4, ABCC9, ABR, ABRA, ACADVL, ACSF3, ACSL4, ADAMTS18,
ADAMTS2, ADGRG4, AFAP1, AFF1, AHNAK, AIRE, AKAP10, ALDH3A2, ALKBH1,
ALOX12, ALX1, AMER1, AMOTL1, AMPD1, ANAPC1, ANKFN1, ANKRD50, ANO3,
AP5M1, APC, ARHGAP23, ARID1A, ARID2, ATF2, ATG16L2, ATP11B, ATP11C, ATP2B1,
ATXN7L1, BAD, BAMBI, BAP1, BCAR1, BCL6, BCL7C, BCR, BRAF, BRD4, BRWD3,
BTBD1, C11orf86, C22orf39, C2CD2L, C5, C5orf42, C6orf222, C9orf131, C9orf57, CA9,
CACNA1I, CALR3, CAPN13, CCDC129, CCDC168, CCDC7, CCDC73, CD163L1, CD84,
CDC42EP1, CDC5L, CDH8, CDKN1A, CENPK, CEP170B, CER1, CFAP65, CFAP99, CHL1,
CHST1, CISD3, CITED2, CLASP2, CLCN7, CLUH, CMPK2, CMTM4, CNNM4, CNOT1,
CNTLN, CNTN6, COL14A1, COL22A1, COL27A1, COL6A3, COPG2, CPEB4, CPXM1, CR2,
CREBBP, CRELD1, CRELD2, CSMD2, CSN3, CSNK1E, CST1, CTC1, CTCFL, CTNND1,
CUL7, CWH43, CYC1, CYHRI, CYP2A7, DAPK2, DCAF11, DCDC1, DENNDIB, DHH,
DIAPH2, DLEC1, DLG3, DMBT1, DMD, DNAH11, DNAH3, DNAH5, DNAH6, DNAH7,
DNAJC6, DNMT3B, DOCK7, DOPEY2, DOT1L, DPY19L1, DRD2, DSCC1, DSG2, DUOX2,
DUOXA2, DUSP28, DYNLT3, DZIP1, E2F2, EIF4G3, ELF3, ELK4, ELP3, ENPP2, EP300,
EPB41, EPHB1, ERICH3, ERMN, ETV5, EXD3, FAAP24, FAM102B, FAM120A, FAM208A,
FAM221A, FAM45A, FAM50A, FAM53A, FAM65A, FANCI, FARP2, FAT3, FBXL5, FFAR3,
FIGN, FIP1L1, FLT3, FMN1, FMR1NB, FNBP4, FOXA1, FREM3, FRMPD2, FSD1L,
GABRA4, GALK2, GGCX, GNPTAB, GPHN, GPR158, GPR179, GPS2, GRAMD4, GRIPAP1,
GRK2, GSDMB, GSTA1, GTF2F1, GUCY1B3, HELZ, HEPHL1, HGF, HIGD2B, HIPK3,
HIRIP3, HMCN1, HOXA6, HPSE, HRAS, HRH4, HS3ST4, HSD17B11, HSF1, HSPA6, HSPB1,
HTT, HUWE1, ICE1, IDH2, IFIT1B, IFT46, IKZF2, IL1RAPL1, IL411, INPPL1, INVS, IRF1,
ITGB4, IZUMO2, KAT2A, KATNAL2, KDM6A, KHSRP, KIAA0586, KIDINS220, KIF15,
KIFC2, KLF5, KLHL3, KLHL34, KMT2A, KMT2B, KMT2D, KRT23, L3MBTL1, LAMA3,
LARP4, LARS2, LNPEP, LOC101928841, LOXHD1, LPP, LRP1, LRRC14, LRRC7, LRRK2,
LSS, LUC7L, MAML3, MAOA, MAP2K2, MAP3K21, MAPK1, MAPRE2, MARK2, MCC,
MCCC2, MCTP2, MED12, MEGF10, MERTK, METAP2, MIPOL1, MLF1, MMACHC,
MRFAP1L1, MSL3, MTA1, MUC16, MYBPC1, MYCBP2, MYH7, N4BP2, NAALADL2,
NANS, NAP1L5, NBEAL1, NCKAP1L, NCOR1, NCOR2, NDST3, NECTIN1, NEGR1, NEK4,
NF1, NF2, NFASC, NFYC, NGLY1, NHEJ1, NHS, NKAP, NLRP14, NMNAT1, NOM1, NPAS2,
NRN1, NRSN1, NSUN5, NUFIP1, NUP214, NUP98, NXPE3, OBSCN, OCRL, OPHN1,
OPLAH, OR2T35, OR4C15, OR52N2, OR6N2, OR6S1, P3H2, P4HB, PAIP2, PAN2, PAPOLB,
PARD6B, PARL, PASD1, PASK, PAX7, PCDHA5, PCF11, PCM1, PDE12, PDGFB, PDK4,
PELP1, PGBD4, PIK3C2B, PIK3C2G, PIK3CA, PIK3CB, PIWIL2, PIWIL3, PLCE1, PLEC,
PLEKHA8, PLEKHG6, PLEKHH3, PLXNB1, PMFBP1, PMPCA, POP1, PPARA, PPP1R12B,
PPP1R2P9, PPP1R35, PPP2CA, PQLC1, PRAMEF12, PRDM4, PRKD1, PRKDC, PROM2,
PRSS22, PRSS47, PSIP1, PSMD13, PTGS1, PTPRC, PYHIN1, RALGAPA2, RANGRF,
RBM15, RBM3, RCOR1, RDH10, RDH11, RDH5, RDX, REPIN1, REPS1, REPS2, RFXANK,
RHO, RHOT1, RIT2, RLIM, RNF115, RNF215, RNFT2, ROCK1, RP2, RPS6KA6, RRS1,
RSAD1, RTN4RL1, RWDD2A, RYR1, S100A4, S1PR5, SAAL1, SAMD9, SBF1, SCRIB,
SDCBP, SENP5, SERPINB6, SETD2, SETDB1, SETX, SEZ6, SF3B1, SFRP1, SFXN4, SGSM3,
SH3GL1, SHANK2, SHROOM2, SI, SIAH3, SIPA1L2, SIRPB2, SIRT2, SIT1, SLC26A4,
SLC2A3, SLC30A8, SLC33A1, SLC38A1, SLC38A2, SLC3A1, SLC6A6, SLC9A4, SLCO4A1,
SLITRK2, SMAP2, SMCR8, SMIM10, SMPD4, SNX13, SOX2, SOX6, SPAG17, SPATA2L,
SPATS1, SPEN, SPICE1, SSH2, ST18, STAG2, STRAP, STRIP2, STT3B, SUPT20HL2,
SUPT6H, SYMPK, SYNE2, SYT1, SZT2, TAF1C, TAF6, TAOK1, TBC1D10C, TCL1B,
TEKT1, TENM2, TERF2IP, TERT, TFAP2C, TGS1, TIGD6, TLN2, TMCC1, TMEM127,
TMEM27, TMEM63A, TNFAIP2, TNRC6A, TNRC6B, TONSL, TOPBP1, TP53, TPGS1, TPK1,
TRAPPC8, TRIB1, TRIM50, TRPC4, TRPC5, TSC1, TSC2, TSNARE1, TTLL3, TTN, TTYH3,
TXNIP, UBE2M, UBR4, UBR5, UGDH, UMODL1, UNC13B, UPK3A, USP22, USP9Y, UTY,
VAV2, VCP, VCPIP1, VWA5B2, VWF, WAPL, WASHC5, WDFY3, WDR48, WDR66,
WDR72, WRNIP1, WTAP, WTIP, XIRP2, XRCC3, XYLT1, YARS2, YTHDC2, ZBTB9,
ZC3H6, ZMYM4, ZMYND19, ZNF280C, ZNF284, ZNF302, ZNF420, ZNF436, ZNF536,
ZNF592, ZNF704, ZNF76, ZNF775, ZSCAN12, ZSWIM5, ZXDC, ZZZ3

TABLE 10
List of genes with SNVs and InDels detected by
the fixed actionable/hotspot panel sequencing.

ACSF3, AHR, ALDH3A2, ANAPC1, ANK2, ANKHD1, AR, ARID1A, ARID1B, ATM, BLM,
BRAF, BRCA1, BRCA2, C17orf97, C3orf70, CASP8, CDKN1A, CDKN2A, CER1, CREBBP,
CTNNB1, CYC1, DIDO1, DNMT3A, EGFR, ELF3, EP300, EPHB1, EPYC, ERBB2, ERBB3,
ERCC2, ESPL1, FBN3, FBXW7, FGFR1, FGFR2, FGFR3, FGFR4, FMN2, FOXA1, HELZ,
HIRIP3, HIST1H1C, HRAS, HRH4, IKZF1, KANSL1, KDM6A, KMT2A, KMT2D, KRAS,
MROH2B, MYC, MYH9, NF1, NFE2L2, NTRK1, NTRK3, PIAS1, PIK3CA, PKHD1, PTEN,
PTPRT, RARS2, RB1, RHOA, RHOB, RXRA, SACS, SF3B1, SF3B3, SOX2, SPOP, STAG2,
SYNE1, TEKT1, TERT, TMEM132D, TNKS, TP53, TSC1, VHL, WDR66, ZFP36L1

TABLE 11
Mutational measurements of each urine/plasma sample in our study.

| | | | | Num | Num | | | | utDNA | | |
|---|---|---|---|---|---|---|---|---|---|---|---|
| | | Sample | Time | Baseline | MRD | | | TFcn | clearance | | |
| PatientID | SampleID | Type | Point | Var | Var | MRD call | TFsm (%) | (%) | (%) | RECIST1.1 | Pathology |
| RZ01 | H009496 | Urine | C1 | 48 | 48 | Positive | 54.10 | 7.10 | 61.20 | PR | non-ypCR |
| RZ01 | H009830 | Urine | C2 | 48 | 48 | Positive | 65.12 | 35.80 | 100.92 | PR | non-ypCR |
| RZ01 | H009928 | Urine | C3 | 48 | 48 | Positive | 58.62 | 44.20 | 102.82 | PR | non-ypCR |
| RZ01 | H010013 | Urine | RC | 48 | 45 | Positive | 3.19 | 0.00 | 3.19 | PR | non-ypCR |
| RZ02 | H009515 | Urine | C1 | 10 | 10 | Positive | 30.92 | 5.40 | 36.32 | SD | non-ypCR |
| RZ02 | H009881 | Urine | RC | 10 | 10 | Positive | 31.74 | 4.10 | 35.84 | SD | non-ypCR |
| RZ03 | H009869 | Urine | C1 | 39 | 38 | Positive | 35.52 | 19.60 | 55.12 | PR | non-ypCR |
| RZ03 | H010302 | Urine | C3 | 39 | 37 | Positive | 28.81 | 18.50 | 47.31 | PR | non-ypCR |
| RZ03 | H010360 | Urine | RC | 39 | 38 | Positive | 28.50 | 23.00 | 51.50 | PR | non-ypCR |
| RZ04 | H010303 | Urine | C1 | 37 | 37 | Positive | 27.02 | 25.50 | 52.52 | SD | non-ypCR |
| RZ04 | H010361 | Urine | RC | 37 | 37 | Positive | 12.27 | 9.10 | 21.37 | SD | non-ypCR |
| RZ05 | H010746 | Urine | C1 | 44 | 44 | Positive | 44.17 | 30.60 | 74.77 | SD | non-ypCR |
| RZ05 | H010866 | Urine | C2 | 44 | 44 | Positive | 43.52 | 30.70 | 74.22 | SD | non-ypCR |
| RZ05 | H010942 | Urine | C3 | 44 | 44 | Positive | 41.60 | NA | 41.60 | SD | non-ypCR |
| RZ05 | H011112 | Urine | C4 | 44 | 44 | Positive | 38.42 | 35.30 | 73.72 | SD | non-ypCR |
| RZ05 | H011322 | Urine | RC | 44 | 44 | Positive | 32.80 | 35.50 | 68.30 | SD | non-ypCR |
| RZ06 | H010887 | Urine | C1 | 30 | 25 | Positive | 3.06 | 3.00 | 6.06 | PR | ypCR |
| RZ06 | H010944 | Urine | C3 | NA | NA | NA | NA | 3.30 | 3.30 | PR | ypCR |
| RZ06 | H011113 | Urine | RC | 30 | 25 | Positive | 3.10 | 3.40 | 6.50 | PR | ypCR |
| RZ07 | H010867 | Urine | C1 | 32 | 32 | Positive | 43.02 | 24.10 | 67.12 | PR | non-ypCR |
| RZ07 | H011099 | Urine | C3 | 32 | 32 | Positive | 45.42 | 26.00 | 71.42 | PR | non-ypCR |
| RZ07 | H011325 | Urine | RC | 32 | 32 | Positive | 58.43 | 23.40 | 81.83 | PR | non-ypCR |
| RZ08 | H010906 | Urine | C1 | 43 | 43 | Positive | 23.51 | 12.60 | 36.11 | PR | ypCR |
| RZ08 | H011945 | Urine | RC | 43 | 7 | Positive | 0.02 | 3.70 | 3.72 | PR | ypCR |
| RZ09 | H011100 | Urine | C1 | 37 | 36 | Positive | 2.27 | 3.40 | 5.67 | PR | ypCR |
| RZ09 | H011270 | Urine | C3 | 37 | 15 | Positive | 0.10 | 3.20 | 3.30 | PR | ypCR |
| RZ09 | H011559 | Urine | C4 | 37 | 4 | Positive | 0.03 | 3.70 | 3.73 | PR | ypCR |
| RZ09 | H011686 | Urine | RC | 37 | 2 | Positive | 0.00 | 2.40 | 2.40 | PR | ypCR |
| RZ10 | H011269 | Urine | C1 | 50 | 17 | Positive | 0.05 | 2.80 | 2.85 | PR | ypCR |
| RZ10 | H011717 | Urine | RC | 50 | 0 | Negative | 0.00 | 5.50 | 5.50 | PR | ypCR |
| RZ11 | H011237 | Urine | C1 | 46 | 46 | Positive | 18.51 | 11.10 | 29.61 | SD | non-ypCR |
| RZ11 | H011615 | Urine | C3 | 46 | 46 | Positive | 15.95 | 10.50 | 26.45 | SD | non-ypCR |
| RZ11 | H011687 | Urine | C4 | 46 | 46 | Positive | 13.19 | 11.00 | 24.19 | SD | non-ypCR |
| RZ11 | H011950 | Urine | RC | 46 | 30 | Positive | 4.95 | 6.80 | 11.75 | SD | non-ypCR |
| RZ12 | H011236 | Urine | C1 | 50 | 8 | Positive | 2.60 | 21.30 | 23.90 | PD | non-ypCR |
| RZ12 | H011342 | Urine | C2 | 50 | 8 | Positive | 2.15 | 16.50 | 18.65 | PD | non-ypCR |
| RZ12 | H011689 | Urine | RC | 50 | 8 | Positive | 2.20 | 17.70 | 19.90 | PD | non-ypCR |
| RZ13 | H011235 | Urine | C1 | 41 | 41 | Positive | 31.42 | 18.40 | 49.82 | PR | ypCR |
| RZ13 | H011619 | Urine | C3 | 41 | 41 | Positive | 31.38 | 18.60 | 49.98 | PR | ypCR |
| RZ13 | H011690 | Urine | RC | 41 | 41 | Positive | 1.94 | 3.00 | 4.94 | PR | ypCR |
| RZ14 | H011288 | Urine | C1 | 22 | 21 | Positive | 5.29 | 4.30 | 9.59 | PR | non-ypCR |
| RZ14 | H011558 | Urine | C2 | 22 | 22 | Positive | 11.86 | 6.10 | 17.96 | PR | non-ypCR |
| RZ14 | H011616 | Urine | C3 | 22 | 22 | Positive | 14.31 | 7.50 | 21.81 | PR | non-ypCR |
| RZ14 | H011719 | Urine | C4 | 22 | 22 | Positive | 10.27 | 5.10 | 15.37 | PR | non-ypCR |
| RZ14 | H200081 | Urine | RC | 22 | 21 | Positive | 29.75 | 63.40 | 93.15 | PR | non-ypCR |
| RZ15 | H011289 | Urine | C1 | 50 | 5 | Positive | 26.63 | 13.30 | 39.93 | CR | ypCR |
| RZ15 | H011561 | Urine | C2 | 50 | 4 | Positive | 16.19 | 6.30 | 22.49 | CR | ypCR |
| RZ15 | H011948 | Urine | C4 | 50 | 4 | Positive | 0.84 | 2.80 | 3.64 | CR | ypCR |
| RZ15 | H200256 | Urine | RC | 50 | 0 | Negative | 0.00 | 3.20 | 3.20 | CR | ypCR |
| RZ16 | H011323 | Urine | C1 | 29 | 29 | Positive | 3.93 | 3.20 | 7.13 | PR | non-ypCR |
| RZ16 | H011617 | Urine | C2 | 29 | 29 | Positive | 4.38 | NA | 4.38 | PR | non-ypCR |
| RZ16 | H011688 | Urine | C3 | 29 | 29 | Positive | 9.87 | 9.60 | 19.47 | PR | non-ypCR |
| RZ16 | H011868 | Urine | C4 | 29 | 29 | Positive | 11.17 | 8.80 | 19.97 | PR | non-ypCR |
| RZ16 | H200082 | Urine | RC | 29 | 29 | Positive | 4.62 | 7.50 | 12.12 | PR | non-ypCR |
| RZ17 | H011560 | Urine | C1 | 22 | 22 | Positive | 34.34 | 26.90 | 61.24 | PR | non-ypCR |
| RZ17 | H011612 | Urine | C2 | 22 | 22 | Positive | 34.94 | 27.40 | 62.34 | PR | non-ypCR |
| RZ17 | H011718 | Urine | C3 | 22 | 22 | Positive | 34.64 | 31.20 | 65.84 | PR | non-ypCR |
| RZ17 | H011869 | Urine | C4 | 22 | 22 | Positive | 33.11 | 41.50 | 74.61 | PR | non-ypCR |
| RZ17 | H200086 | Urine | RC | 22 | 22 | Positive | 28.05 | 33.40 | 61.45 | PR | non-ypCR |
| RZ18 | H011613 | Urine | C1 | 39 | 39 | Positive | 7.85 | 3.60 | 11.45 | CR | ypCR |
| RZ18 | H011691 | Urine | C2 | 39 | 39 | Positive | 23.51 | 3.80 | 27.31 | CR | ypCR |
| RZ18 | H011871 | Urine | C3 | 39 | 39 | Positive | 20.73 | 2.90 | 23.63 | CR | ypCR |
| RZ18 | H011944 | Urine | RC | 39 | 4 | Positive | 0.04 | 2.60 | 2.64 | CR | ypCR |
| RZ19 | H011568 | Urine | C1 | 50 | 7 | Positive | 1.70 | 4.50 | 6.20 | SD | non-ypCR |
| RZ19 | H011618 | Urine | C2 | 50 | 4 | Positive | 2.04 | 3.10 | 5.14 | SD | non-ypCR |
| RZ19 | H011949 | Urine | RC | 50 | 4 | Positive | 0.77 | 3.10 | 3.87 | SD | non-ypCR |
| RZ20 | H011611 | Urine | C1 | 43 | 43 | Positive | 5.36 | 5.00 | 10.36 | CR | ypCR |
| RZ20 | H011720 | Urine | C2 | 43 | 43 | Positive | 6.36 | 5.60 | 11.96 | CR | ypCR |
| RZ20 | H011943 | Urine | C4 | 43 | 0 | Negative | 0.00 | 2.60 | 2.60 | CR | ypCR |
| RZ20 | H200257 | Urine | RC | 43 | 1 | Negative | 0.00 | 3.40 | 3.40 | CR | ypCR |
| RZ01 | H200357 | Plasma | C1 | 48 | 0 | Negative | 0.00 | 0.00 | 0.00 | PR | non-ypCR |
| RZ01 | H200365 | Plasma | RC | 48 | 0 | Negative | 0.00 | 0.00 | 0.00 | PR | non-ypCR |
| RZ02 | H200369 | Plasma | C1 | 10 | 1 | Negative | 0.00 | 0.00 | 0.00 | SD | non-ypCR |
| RZ02 | H200373 | Plasma | RC | 10 | 0 | Negative | 0.00 | 0.00 | 0.00 | SD | non-ypCR |
| RZ03 | H200377 | Plasma | C1 | 39 | 0 | Negative | 0.00 | 0.00 | 0.00 | PR | non-ypCR |
| RZ03 | H200385 | Plasma | RC | 39 | 2 | Positive | 0.01 | 0.00 | 0.01 | PR | non-ypCR |
| RZ04 | H200391 | Plasma | C1 | 37 | 1 | Negative | 0.00 | 0.00 | 0.00 | SD | non-ypCR |
| RZ04 | H200393 | Plasma | RC | 37 | 20 | Positive | 0.07 | 0.00 | 0.07 | SD | non-ypCR |
| RZ05 | H200401 | Plasma | C1 | 44 | 1 | Negative | 0.00 | 0.00 | 0.00 | SD | non-ypCR |
| RZ05 | H200409 | Plasma | RC | 44 | 0 | Negative | 0.00 | 0.00 | 0.00 | SD | non-ypCR |
| RZ06 | H200411 | Plasma | C1 | NA | NA | NA | NA | 0.00 | 0.00 | PR | ypCR |
| RZ06 | H200419 | Plasma | RC | NA | NA | NA | NA | 0.00 | 0.00 | PR | ypCR |
| RZ07 | H200421 | Plasma | C1 | 32 | 1 | Negative | 0.00 | 0.00 | 0.00 | PR | non-ypCR |
| RZ07 | H200428 | Plasma | RC | 32 | 2 | Positive | 0.01 | 0.00 | 0.01 | PR | non-ypCR |
| RZ08 | H200430 | Plasma | C1 | 43 | 0 | Negative | 0.00 | 0.00 | 0.00 | PR | ypCR |
| RZ08 | H200436 | Plasma | RC | 43 | 1 | Negative | 0.00 | 0.00 | 0.00 | PR | ypCR |
| RZ09 | H200440 | Plasma | C1 | 37 | 35 | Positive | 0.27 | 0.00 | 0.27 | PR | ypCR |
| RZ09 | H200446 | Plasma | RC | 37 | 0 | Negative | 0.00 | 0.00 | 0.00 | PR | ypCR |
| RZ10 | H200448 | Plasma | C1 | 50 | 0 | Negative | 0.00 | 0.00 | 0.00 | PR | ypCR |
| RZ10 | H200456 | Plasma | RC | 50 | 0 | Negative | 0.00 | 0.03 | 0.03 | PR | ypCR |
| RZ11 | H200460 | Plasma | C1 | 46 | 3 | Positive | 0.02 | 0.00 | 0.02 | SD | non-ypCR |
| RZ11 | H200468 | Plasma | RC | 46 | 2 | Positive | 0.03 | 0.00 | 0.03 | SD | non-ypCR |
| RZ12 | H200475 | Plasma | C1 | 3 | 1 | Negative | 0.00 | 0.00 | 0.00 | PD | non-ypCR |
| RZ12 | H200479 | Plasma | RC | 3 | 0 | Negative | 0.00 | 0.00 | 0.00 | PD | non-ypCR |
| RZ13 | H200481 | Plasma | C1 | 41 | 29 | Positive | 0.22 | 0.00 | 0.22 | PR | ypCR |
| RZ13 | H200489 | Plasma | RC | 41 | 0 | Negative | 0.00 | 0.03 | 0.03 | PR | ypCR |
| RZ14 | H200495 | Plasma | C1 | 22 | 0 | Negative | 0.00 | 0.00 | 0.00 | PR | non-ypCR |
| RZ14 | H200501 | Plasma | RC | 22 | 0 | Negative | 0.00 | 0.00 | 0.00 | PR | non-ypCR |
| RZ15 | H200502 | Plasma | C1 | 50 | 0 | Negative | 0.00 | 0.00 | 0.00 | CR | ypCR |
| RZ15 | H200508 | Plasma | RC | 50 | 0 | Negative | 0.00 | 0.00 | 0.00 | CR | ypCR |
| RZ16 | H200510 | Plasma | C1 | 29 | 0 | Negative | 0.00 | 0.00 | 0.00 | PR | non-ypCR |
| RZ16 | H200514 | Plasma | RC | 29 | 1 | Negative | 0.00 | 0.00 | 0.00 | PR | non-ypCR |
| RZ17 | H200517 | Plasma | C1 | 22 | 0 | Negative | 0.00 | 0.00 | 0.00 | PR | non-ypCR |
| RZ17 | H200524 | Plasma | RC | 22 | 0 | Negative | 0.00 | 0.00 | 0.00 | PR | non-ypCR |
| RZ18 | H200528 | Plasma | C1 | 39 | 0 | Negative | 0.00 | 0.00 | 0.00 | CR | ypCR |
| RZ18 | H200536 | Plasma | RC | 39 | 1 | Negative | 0.00 | 0.00 | 0.00 | CR | ypCR |
| RZ19 | H200537 | Plasma | C1 | 50 | 0 | Negative | 0.00 | 0.00 | 0.00 | SD | non-ypCR |
| RZ19 | H200543 | Plasma | RC | 50 | 0 | Negative | 0.00 | 0.04 | 0.04 | SD | non-ypCR |
| RZ20 | H200545 | Plasma | C1 | 43 | 2 | Positive | 0.01 | 0.00 | 0.01 | CR | ypCR |
| RZ20 | H200552 | Plasma | RC | 43 | 0 | Negative | 0.00 | 0.00 | 0.00 | CR | ypCR |

C1 = cycle 1; C2 = cycle 2; C3 = cycle 3; C4 = cycle 4; RC = radical cystectomy; MRD = minimal residual disease; TFsm = tumor fraction estimate based on somatic mutations; TFcn = tumor fraction estimate based on copy numbers; CR = complete response; PR = partial response; SD = stable disease; PD = progressive disease; ypCR = yield-pathological complete response.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-69. (canceled)

70. A method for detecting a presence or an absence of minimal residual disease (MRD) in a subject, comprising:
(a) assaying deoxyribonucleic acid (DNA) molecules from a first biological sample obtained or derived from said subject at a first time point;
(b) detecting a set of biomarkers from said DNA molecules based at least in part on said assaying of (a), wherein said set of biomarkers comprise differentially expressed markers or variants;
(c) generating a plurality of probe nucleic acids that are customized for said subject, wherein said probe nucleic acids comprises sequences of at least a subset of said set of biomarkers;
(d) using said plurality of probe nucleic acids, sequencing cell free deoxynucleic acids (cfDNA) from a second biological sample obtained or derived from said subject at a second time point to detect the presence or absence of said subset of said set of biomarkers, wherein said sequencing is performed at a depth of at least about 80×;
(e) sequencing nucleic acids obtained or derived from said subject at least part by using whole genome sequencing to determine a copy number of at least one region of a genome of a subject; and
(f) computer processing said subset of said set of biomarkers and said copy number of said at least one region of a genome to detect said presence or absence of minimal residual disease (MRD) in a subject.

71. The method of claim 70, wherein said first or second biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a saliva sample, tissue biopsy, pleural fluid sample, peritoneal fluid sample, amniotic fluid sample, cerebroshinal fluid sample, lymphatic fluid sample, sweat sample, tear sample, semen sample, or any derivative thereof, and any combination thereof.

72. The method of claim 70, wherein said first or second biological sample comprises said plasma sample.

73. The method of claim 70, wherein said first or second biological sample comprises said urine sample.

74. The method of claim 70, wherein at least one of said DNA molecules are assayed using whole exome sequencing to produce nucleic acid sequencing reads.

75. The method of claim 70, wherein said whole genome sequencing of (e) further comprises low-pass whole genome sequencing.

76. The method of claim 70, wherein said sequencing of (d) is performed at a depth of at least about 1,000×.

77. The method of claim 70, wherein said sequencing of (e) further comprises sequencing nucleic acids of a sample taken at said first time point, and sequencing nucleic acids of a sample taken at a second time point.

78. The method of claim 77, further comprising comparing results of said sequencing of nucleic acids of said sample taken at said first time point and said sequencing of said nucleic acids of said sample taken at said second time point to determine said copy number of at least one region of a genome of a subject.

79. The method of claim 70, wherein said cancer is selected from the group consisting of: genitourinary cancer, breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof.

80. The method of claim 79, wherein said cancer comprises said bladder cancer.

81. The method of claim 70, wherein said biological sample is obtained or derived from said subject after receiving a therapy for said cancer.

82. The method of claim 70, further comprising identifying a clinical intervention to treat said MRD in said subject, based at least in part on said detected presence or said absence of said cancer.

83. The method of claim 82, wherein said clinical intervention is selected from the group consisting of: surgical resection, chemotherapy, radiotherapy, immunotherapy, adjuvant therapy, neoadjuvant therapy, androgen deprivation therapy, and a combination thereof.

84. The method of claim 82, further comprising administering said clinical intervention to said subject thereby treating said MRD in said subject.

85. The method of claim 70, wherein said set of biomarkers comprises one or more members selected from the group consisting of genes listed in Table 1.

86. The method of claim 70, wherein said set of biomarkers comprises one or more members selected from the group consisting of genes listed in Table 7.

87. The method of claim 70, wherein said set of biomarkers comprises one or more members selected from the group consisting of genes listed in Table 8.

88. The method of claim 70, wherein said set of biomarkers comprises one or more members selected from the group consisting of genes listed in Table 9.

89. The method of claim 70, wherein (d) further comprises sequencing using a fixed plurality of probes, wherein said probes of said fixed plurality of probes comprise probes that do not comprise sequences of said subset of said set of biomarkers.

90. The method of claim 89, wherein said fixed plurality of probes comprise one or more members selected from the group consisting of genes listed in Table 10.

91. The method of claim 70, wherein said set of biomarkers comprises tumor-associated alterations selected from the group consisting of: single nucleotide variants (SNVs), insertions or deletions (indels), and rearrangements.

92. The method of claim 70, further comprising, based at least in part on said sequencing of (e), detecting a copy number variation or a copy number loss.

93. The method of claim 70, further comprising determining, among said set of biomarkers, a mutant allele frequency of a set of somatic mutations.

94. The method of claim 93, further comprising determining a circulating tumor DNA (ctDNA) fraction of said cancer of said subject, based at least in part on said set of mutant allele frequencies.

95. The method of claim 93, further comprising determining a tumor mutational burden (TMB) of said cancer of said subject, based at least in part on said set of mutant allele frequencies.
